(19) United States
(12) Patent Application Publication
(10) Pub. No.: US 2002/0042394 A1
(43) Pub. Date: Apr. 11, 2002

(54) Cobalamin compounds useful as antibiotic agents and as imaging agents

(76) Inventors: Henricus P.C. Hogenkamp, Roseville, MN (US); Douglas A. Collins, Rochester, MN (US)

Correspondence Address: KING & SPALDING, 191 PEACHTREE STREET, N.E., ATLANTA, GA, 30303-1763, US

(21) Appl. No.: 09/873,164

(22) Filed: May 31, 2001

Related U.S. Application Data
(60) Provisional application No. 60/208,148, filed on May 31, 2000.
(60) Provisional application No. 60/267,543, filed on Feb. 9, 2001.

(51) Int. Cl. A61K 31/714; A61K 31/555; C07H 23/00

(52) U.S. Cl. 514/53; 514/185; 540/145; 536/26.44

(57) ABSTRACT

The invention provides cobalamin derivatives linked to an antibiotic and/or an imaging agent, as well as pharmaceutical compositions comprising the compounds and methods for using the compounds in treatment or diagnosis of a microbial infection.

This application claims priority to U.S. provisional application No. 60/208,148, filed on May 31, 2000 and U.S. provisional application No. 60/267,543, filed on Feb. 9, 2001.

### FIELD OF THE INVENTION

This invention provides compounds, compositions and methods for treating microbial infection.

### BACKGROUND OF THE INVENTION

Antibiotics are low-molecular weight antimicrobial agents that are produced as secondary metabolites by microorganisms that inhabit soil. For instance, Penicillium and Cephalosporium produce beta-lactam antibiotics (e.g. penicillin, cephalosporin and their relatives). Actinomycetes (e.g. the Streptomyces species) produce tetracyclines, aminoglycosides (i.e. streptomycin and its analogs), macrolides (i.e. erythromycin and its analogs), chloramphenicol, ivermectin, rifamycins and most other clinically-useful antibiotics that are not beta-lactams. Bacillus species (e.g. B. polyrnyxa and Bacillus subtills) produce 15 polypeptide antibiotics (e.g. polymyxin and bacitracin), while B. cereus produces zwittermicin.

The modem era of antibiotic therapy began with Fleming's 1929 discovery of penicillin and Domagk's 1935 discovery of synthetic sulfonamides. Spurred by the need for antibacterial drags during World War II, penicillin was isolated, purified and injected into experimental animals. The substance was found to not only cure infections, but also to possess low toxicity. This finding marked the beginning of the era of antibiotic use in human drug therapy and the intense search for similar antimicrobial agents of low toxicity that could be used to treat infectious diseases. The rapid isolation of streptomycin, chloramphenicol and tetracycline followed and these and several other antibiotics were in clinical usage by the 1950's.

Antibiotics are used therapeutically to treat bacterial infections. Several types of antibiotics, classified according to their mechanism of action, are currently employed. The known types of antibiotics include, e.g. cell wall synthesis inhibitors, cell membrane inhibitors, protein synthesis inhibitors and inhibitors that bind to or affect the synthesis of DNA or RNA.

Antibiotics

Cell wall synthesis inhibitors, such as beta lactam antibiotics, generally inhibit some step in the synthesis of bacterial peptidoglycan. Penicillin is generally effective against non-resistant streptococcus, gonococcus and staphylococcus. Amoxycillin and Ampicillin have broadened spectra against Gram-negative bacteria. Cephalosporins are generally used as penicillin substitutes, against Gram-negative bacteria and in surgical prophylaxis. Monobactams are generally useful for the treatment of allergic individuals.

Cell membrane inhibitors disorganize the structure or inhibit the function of bacterial membranes. Polymyxin, produced by Bacillus polymyxis, is a cell membrane inhibitor that is effective mainly against Gram-negative bacteria and is usually limited to topical usage.

Protein synthesis inhibitors include the tetracyclines, chloramphenicol, the macrolides (e.g. erythromycin) and the aminoglycosides (e.g. streptomycin). Aminoglycosides have been used against a wide variety of bacterial infections caused by Gram-positive and Gram-negative bacteria. Streptomycin has been used extensively as a primary drag in the treatment of tuberculosis. Gentamicinis active against many strains of Gram-positive and Gram-negative bacteria, including some strains of Pseudomonas aeruginosa. Kanamycin is active at low concentrations against many Gram-positive bacteria, including penicillin-resistant staphylococci.

The tetracyclines are protein synthesis inhibitors that consist of eight related antibiotics that are all natural products of Streptomyces, although some can now be produced semisynthetically. Tetracycline, chlortetracycline and doxycycline are the best known. The tetracyclines are broad-spectrum antibiotics with a wide range of activity against both Gram-positive and Gram-negative bacteria. Tetracyclines have some important uses, such as in the treatment of Lyme disease.

Chloramphenicol is a protein synthesis inhibitor that has a broad spectrum of activity but it exerts a bacteriostatic effect. It is effective against intracellular parasites such as the rickettsiae. It is infrequently used in human medicine except in life-threatening situations (e.g. typhoid fever). Macrolide antibiotics, such as erythromycin, are protein synthesis inhibitors that are active against most Gram-positive bacteria.

Some antibiotics affect the synthesis of DNA or RNA or can bind to DNA or RNA so that their messages cannot be read. For example, nalidixic acid is a synthetic quinoloid antibiotic that is active mainly against Gram-negative bacteria. The main use of nalidixic acid is in treatment of lower urinary tract infections (LUTI). In addition, the rifamycins has greater bactericidal effect against the bacteria that causes tuberculosis than other anti-tuberculosis drugs and is also useful for treatment of tuberculosis meningitis and meningitis caused by Neisseria meningitidis.

Finally, competitive inhibitors are generally synthetic antibiotics that are growth factor analogs. Growth factor analogs are structurally similar to bacterial growth factors, but do not fulfill their metabolic functions in cells. For example, sulfonamides have been extremely useful in the treatment of uncomplicated UTI caused by E. coli and in the treatment of meningococcal, meningitis.

Suitable antibiotic agents are disclosed, e.g. in Physician's Desk 30 Reference (PDR), Medical Economics Company (Montvale, N.J.), (53^{rd} Ed.), 1999; Mayo Medical Center Formulary, Unabridged Version, Mayo Clinic (Rochester, Minn.), January 1998; Merck Index An Encyclopedia of Chemicals, Drugs and Biologicals, (11^{th} Ed.), Merck & Co., Inc. (Rahway, N.J.), 1989; University of Wisconsin Antimicrobial Use Guide, http://www.medsch.wisc.edu/clinsci/ 5amcg/amcg.html; Introduction on the Use of the Antibiotics Guideline, of Specific Antibiotic Classes, Thomas Jefferson University, http://jeffiine.tju.edu/CWIS/OAC/antibiotics guide/ intro.html; and references cited therein.

Suitable antibiotics include, e.g. aminoglycosides, β-lactam antibiotics, cephalosporius, macrolides, miscellaneous antibiotics, penicillins, tetracyclines, antifungals, antimalarial agents, antituberculosis agents, antivirals, leprostatics, miscellaneous anti-infectives, quinolones, sulfonamides, urinary anti-infectives, nasal antibiotics, opthalmic antibiotics, opthalmic antivirals, opthalmicquinalones, opthalmic sulfonamides, skin and mucous membrane antibiotics, skin and mucous membrane antifungals, skin and mucous membrane antivirals, skin and mucous membrane miscellaneous anti-infectives, skin and mucous membranescabicides and pedulicides, skin and mucous membrane antineoplasts, nitrofurans and oxazolidinones. Physician's Desk Reference (PDR), Medical Economics Company (Montvale, N.J.), (53^{rd} Ed.), 1999 and Mayo Medical Center Formulary: Unabridged Version, Mayo Clinic (Rochester, Minn.), January 1998.

Aminoglycosides include, for example, Amikacin (amikacin sulfate); Craramyein (gentamicin sulfate); Nebcin (tobramycin sulfate); Netromycin (netilmicin sulfate); Streptomycin Sulfate; and TOBI (tobramycin).

β-Lactam antibiotics include, for example, Azactam (aztreonam); Cefotan (cefotetan); Lorabid (loracarbef); Mefoxin (cefoxitin); Merrem (meropenem); and Primaxin (imipenem and cilastatin for injectable suspension).

Cephalosporins include, for example, Ancef (cefazolin); Ceclor (cefaclor); Cedax (ceffibuten); Cefizox (ceffizoxime sodium); Cefobid (cefoperazone sodium); Ceftin (cefuroxime axetil); Cefzil (cefprozil); Ceptaz (ceftazidime); Claforan (cefotaxime); Duricef (cefadroxil monohydrate); Fortaz (ceftazidime); Keflex (cephalexin); Keftab (cephalexin HCl); Kefurox (cefuroxime); Kefzol (cefazolin); Mandol (cefamandole nafate); Maxipime (cefepime HCl); Monocid (cefonicidsodium); Omnicef (cefdinir); Rocephin (ceftriaxone); Suprax (cefixime); Tazicef (ceftazidime); Tazidime (ceftazidime); Vantin (cefpodoxime proxetil); and Zinacef5(cefuroxime).

Macrolides include, for example, Biaxin (clarithromycin); Dynabac (dirithromycin); E.E.S. 200 (Erythromycin Ethylsuccinate); E.E.S. 400 (Erythromycin Ethylsuccinate); EryPed 200 (Erythromycin Ethylsuccinate); EryPed 400 (Erythromycin Ethylsuccinate); EryTab (Erythromycin delayed-release tablets); Erythrocin Stearate (Erythromycin stearate); Ilosone (erythromycinestolate); PCE Dispertab (erythromycin particles in tablets); Pediazole(erythromycin ethylsuccinate and sulfisoxazole acetyl for oral suspension); Tao (troleandomycin); Zithromax (azithromycin); and Erythromycin.

Miscellaneous antibiotics include, for example, Cleocin HCl (clindamnycin hydrochloride); Cleotin Phosphate (elindamycin phosphate); Coly-Mycin M (colistimethate sodium); and Vancocin HCl (vancomycin hydrochloride).

Penicillins include, for example, Amoxil (amoxicillin); Augmentin (amoxicillin/clavulanate potassium); Bicillin C-R 900/300 (Penicillin G benzathine and Penicillin G procaine suspension); Bicillin C-R (Penicillin G benzathine and Penicillin G procaine suspension); Bicillin L-A (Penicillin G benzathine suspension); Geoeillin (carbencillin indanyl sodium); Mezlin (sterile mezlocillinsodium); Omnipen (ampicillin); Pen-Vee K (penicillin V potassium); Pfizerpen (penicillin G potassium); Pipracil (piperacillin sodium); Speetrobid (bacampicillin-HCl); Ticar (tiearcillin disodium); Timentin (ticarcillin disodium and clavulanate potassium); Unasyn (ampicillin sodium/sulbactam sodium); Zosyn (piperacillin sodium and tazobactam sodium); and Dicloxacillin Sodium.

Tetracyclines include, for example, Achromycin V (tetracycline HCl); Declomycin (demeclo-cycline HCl); Dynacin (minocylcine HCl); Minocin (minocycline hydrochloride); Monodox (Doxycycline monohydrate capsules); Terramycin (oxytetracyline); Vectrin (minocycline hydrochloride); Vibramycin Calcium (doxycycline sodium); Vibramycin Hyclate (doxycycline hyclate);Vibramycin Monohydrate (doxycycline monohydrate); VibraTabs (doxycycline-hydrate); Declomycin (demeclocycline HCl); Vibramycin (doxycycline); Dynacin(Minocyline HCl); Terramycin (oxytetracycline HCl); Achromycin V capsules5 (tetracycline HCl); Linco-mycins; and Cleotin HCl (clindamycin HCl).

Antifungals include, for example, Abelcet (amphotericin B lipid complex); AmBisome (arnphotericin B); Amphotec (amphotericin B cholesterol sulfatecomplex); Ancobon (flucytosine); Diflucan (fluconazole); Fulvicin P/Gamma (ultramicrosize griseofulvin); Fulvicin P/G 165 and 330 (ultramicrosize griseoffilvin); Grifulvin V (griseofulvin); Gals-PEG (gxiseofulvin ultramicrosize); Lamisil (terbinafine hydrochloride); Nizoral (ketoconazole); Amphotericin B; Lotrimin (clotrimazole); Dapsone tablets (dapsone); Diflucan (fluconazole); Monistat-Derm cream (miconazole); Mycostalin Crc .am (nystatin); and Sporanox (itraconazole).

Antimalarial agents include, for example, Aralen hydrochloride (chloroquine HCl); Aralen phosphate (chloroquine phosphate); Dataprim (pyrimethamine); Ladam (mefloquine HCl); and Plaquenil (hydroxychloroqnine sulfate).

Antituberculosis agents include, for example, Capastat sulfate (capreomycinsulfate); Myambutol (ethambutol hydrochloride); Mycobutin (rifabutin capsules); Nydrazid (isoniazid injection); Paser (aminosalicylic acid); Prifiin (rifapentine); Pyrazinamide tablets (pyrazinamide); Rifadin (rifampin capsules); Rifadin IV(rifampin for injection); Rifamate (rifampin and isoniazid); Rifater (rifampin,isoniazid and pyrazinamide); Seromycin (cycloserine capsules); Streptomycin-Sulfate; Tice BCG (BCG vaccine); Cycloserine (seromycin capsules); Urised (Methenamine); and Trecator-SC (ethionamide tablets).

Antivirals include, for example, Alferon N (interferon alfa-n3); Crixivan (indinavir sulfate); Cytovene (ganciclovir); Cytovene-IV (ganciclovir sodium); Epivir (lamivudine); Famvir (famciclovir); Flumadine (rimantadine HCl); Foscavir (foscamet sodium); Hivid (zalcitabine); Intron A (interferon alfa-2b); Invirase (saquinavir mesylate); Norvir (ritonavir); Rebetron combination therapy, which contains Rebetrol (ribavirin) and Intron A (inteferon alfa-2b); Rescriptor (delavirdine mesylate); Retrovir (ziduvudine); Retrovir IV (ziduvudine); Symmetrel (amantadine HCl); Synagis (palivizumab); Valtrex (valacyclovir HCl); Videx (didanosine); Viracept (nelfinavir mesylate); Viramune (nevirapine); Virazole (ribavirin); Vistide (cidofovir); Zerit (stavudine (d4T)); Symmetrel Syrup(amantadine HCl); Combivir Tablets (lamiduvine); and Zovirax (acyclovir).

Leprostatics include, for example, Dapsone Tablets (dapsone).

Miscellaneous anti-infectives include, for example, Daraprim(pyrimethamine); Flagyl 375 (metronidazole); Flagyl ER Tablets (metronidazole); Flagyl I.V. (metronidazole); Furoxone (furazolidone); Mepron (atovaquone); and Neutrexin (tfimetrexate glucuronate).

Quinolones include, for example, Cipro (ciprofloxacin HCl); Floxin(ofloxacin); Levaquin (levofloxacin); Mazaquin (lomefioxacin HCl); Noroxin(norfloxacin); Penetrex (enoxacin); Raxar (grepafloxacin HCl); Trovan (trovafioxacin mesylate); and Zagam (sparfloxacin).

Sulfonamides include, for example, Bactrim.(trimethoprim and sulfamethoxazole); Bactrim DS (Irimethoprim and sulfamethoxazole double strength); Pediazole (erythromycin ethylsuccinate and sulfisoxazole acetyl); Septra(trimethoprim and sulfamethoxazole); Septra DS (trimethoprim and sulfamethoxazole); Co-Trimoxazole, Sulfadiazine, Battrim I.V. Infusion (sulfamethoxazole); Sulfapyridine and Pediazole (erythromycin ethylsuccinate and sulfisoxazole acetyl).

Urinary anti-infectives include, for example, Furadantin (nitrofurantoin); Macrobid (nitrofurantoin monohydrate macrocrystals); Macrodantin (nitrofurantoin macrocrystals); Monurol Sachet (fosfomycin tromethamine); NegGram Caplets(nalidixic acid); Septra (trimethoprim and sulfamethoxazole); Septra DS(trimethoprim and sulfamethoxazole); Urised (a combination of the antisepticsmethenamine, methylene blue, phenyl salicylate, benzoic acid and parasympatholytics (atropine sulfate) hyoscyamine); Urobiotic-250 Capsules (oxytetracycline HCl, sulfamethizole and phenazopyridine HCl); and Uroqid Acid No. 2 Tablets (methenamine mandelate).

Nasal antibiotics include, for example, Bactroban (mupirocin).

Opthalmic antibiotics include, for example, Chloromycetin opthahnic (chloramphenical); Cortisporin (neomycin and polymyxin [3 sulfates and hydrocortisone acetate cream); Ilotycin (erythromycin opthalmic ointment); NeoDecadron (neomycin sulfate—dexamethasone sodium phosphate); Polytrim (tfimethoprim and polythyxin [3 sulfate opthalmic solution); Terra-Cortril (oxytetracycline HCl and hydrocortisone acetate); Terramycin (oxytetracycline); and TobraDex (tobramycin and dexamethasone opthalmic suspension and ointment).

Opthalmic antivitals includes, for example, Vita-A opthalmic ointment, (vidatabine).

Opthalmic quinalones include, for example, Chibroxin (norfloxacinopthalmic solution; Ciloxan opthalmic solution, (Ciprofloxacin HCl); Ciloxan opthalmic ointment, (Ciprofloxacin HCl); and Ocuflox opthalmic solution (ofioxacin).Opthalmic sulfonamides include, for example, Blephamide opthalmicointment (sulfacetamide sodium and prednisolone acetate); and Blephamideopthalmic suspension (sulfacetamide sodium and predrdsolone acetate).

Skin and mucous membrane antibiotics include, for example, A/T/S (erythromycin); Bactroban (mupirocin); Benzamycin (erythromycin-benzoyl peroxide topical gel); Betadine (povidone-odine); Cleotin T (clindamy cinphosphate topical solution); Clindets (clindamycin phosphate pledgets); Cortispofin(neomycin, polymyxin B sulfates and hydrocortisone acetate cream); Emgel (erythromycin); Erycette (erythromycin topical solution); Garamycin (gentamicin sulfate); Klaron (sodium sulfacetamide lotion); Mycostatin (nystatin cream); Theramycin Z (erythromycin topical solution); T-Stat (erythromycin); Chloromycetin (chloramphenicol opthalmic ointment); Cortisporin (neomycin and polymyxin B sulfates, bacitracin zinc and hydrocortisone opthalmic ointment); Ilotycin (erythromycin); NeoDeeadron (neomycin sulfate-dexamethasone sodium phosphate); Polytrim (trimethoprim and polymyxin B sulfate); Terra-Cortril (oxytetracycline HCl and hydrocortisone acetate); Terramycin (oxytetracycline); and TobraDex (tobramycin and dexamethasone opthalmic suspension and ointment).

Skin and mucous membrane antifingals include, for example, Exelderm (sulconazole nitrate); Fungizone (amphotericin B oral suspension); Lamisil (terbinafine hydrochloride cream); Loprox (ciclopiroxolamine); Lotrimin (clotrimazole); Lotrisone (clotrimazole and betamethasone diproprionate); Mentax(butenafine HCl); Monistat-Denn (miconazole nitrate); Mycelex (clotrimazole);Mycostatin (nystatin); Naffin (nattifine HCl); Nizoral Ocetoconazole); Nystop (nystatin); Oxistat (oxiconazole nitrate); Selsun Rx (2.5% selenium sulfide lotion); and Spectazole (econazole nitrate).

Skin and mucous membrane antivirals include, for example, Denavir(enciclovir cream); and Zovirax (acyclovir).

Skin and mucous membrane miscellaneous anti-infectives include, for example, Benzashave Coenzoyl peroxide); Betadine (povidone-iodine); Betasept

(chlorhexidine gluconate); Cetaphil (soap substitute); Clorpactin WCS-90 (sodium oxychlorosene); Dapsone Tablets (dapsone); Desquam-E Coenzoyl peroxide); Desquam-X (benzoyl peroxide); Hibiclens (chlorhexidine gluconate); Hibistat(ehlorhexidine gluconate); Impregon (tetrachlorosalicylanilide 2%); MetroCream (metronidazole); MetroGel (metronidazole); Noritate (metronidazole); pHisoHex (hexachlorophene detergent cleanser); Sulfacet-R (sodium sulfacetarnide 10% and sulfur 5%); Sulfamylon (materfide acetate); Tfiaz Coenzoyl peroxide); and Vanoxide-HC Coenzoyl peroxide hydrocortisone).

Skin and mucous membrane scabicides and pedulicides include, for example, Acticin (permethrin); Elimite (permnethrin); Eurax (crotamiton); and Lindane Lotion USP 1% (lindane).

Skin and mucous membrane antineoplasts include, for example, Efudex (fluorouracil); and Fluoroplex.

Nitrofurans include, for example, Furadantin Oral Suspension (nitrofurantoin).

Oxazolidinones include, for example, Zyvox (linezolid).

It is appreciated that those skilled in the art understand that the antibiotic useful in the present invention is the biologically active compound present in any of the antibiotic formulations disclosed above. For example, Azactam (aztreonam) is typically available as an injectable solution. The antibiotic agent, however, is (z)-2-[[[(2-amino-4-thiazolyl)-[[(2S,-3S)-2-methyl-4-oxo-1-sulfo-3-azetidinyl]carbamoyl]methylene]amino]oxy]-2-methyl-propionic acid. Physician's Desk Reference (PDR), Medical Economics Company (Montvale, N.J.),(53^{rd} Ed.), pp. 820-823, 1999.

Amikacin is commercially available from Elkins-Sinn and is D-streptamine, O-3-amino-3-deoxy-α-D-glucopyranosyl-(1→6)-O-6-deoxy-α-z-D-gluco-pyranosyl-(1→4)]-N′-(4-amino-2 hydroxy-1-oxobutyl)-2-deoxy-,1 (S)-, sulfate (1:2) (salt).

Garamycin (gentamicin sulfate) is commercially available from Schering.

Nebcin (tobramycin sulfate) is commercially available from Lilly and is O-3-amino-3-deoxy-α-D-glucopyranosyl-(→4)-O-[2,6-diamino-2,3-6-trideoxy-α-D-ribo-hexopyranosyl-(1→6)]-2-deoxy-L-streptamine, sulfate (2:5) (salt).

Netromycin (netilmicin sulfate) is commercially available from Schering and is O-3-deoxy-4-C-methyl-3-(methylamino)-β-L-ara-binopyranosyl-(1→4)-O-[2,6-diamino-2,3,4,6-tetradeoxy-α-D-glycero-hex-4-enopyransyl-(1→6)-2-deoxy-N³-ethyl-L-streptamine sulfate (2:5) salt.

Streptomycin Sulfate is commercially available from Pfizer and is D-Streptamine, (1→4)-N,N′-bis(arninoiminomethyl)-O-2-deoxy-2-(methylamino)-α-L-glucopyranosyl-(1→2)-O-5-deoxy-3-C-formyl-L-α-lyxo-furanosyl sulfate (2:3) salt.

TOBI (tobramycin) is commercially available from Pathogenesis Corporation and is O-3-amino-3-deoxy-α-D-glucopyranosyl-(1→4)-O-[2,6-diamino-2,3,6-trideoxy-a-D-ribohexopyranosyl-(1-6)]-2-deoxy-L-streptamine.

Azactam (aztreonam) is commercially available from Bristol-Myers Squibb and is (Z)-2-[[[(2-amino-4-thiazolyl)[[(2S,-3S)-2-methyl-4-oxo-1-sulfo-3-azetidinyl]carbamoyl]-methylene]amino]oxy]-2-methylpropionic acid.

Cefotan (cefotetan) is commercially available from Zeneca and is [6R-(6a,7a)]-7-[[[4-(2-amino-1-carboxy-2-oxoethylidene)-1,3-dithietan-2-yl]carbonyl]-amino]-7-methoxy-3-[[(1 -methyl-1H-tetrazol-5-yl)thio]methyl]-8-oxo-5-thia-1-azabicyclo-[4.2.0]-oct-2-ene-2-carboxylic acid disodium salt.

Lorabid (loracarbef) is commercially available from Lilly and is (6R,7S)-7-[R-2-amino-2-phenylacetamido]-3-chloro-8-oxo-1-azabicyclo-[4.2.0]-oct-2-ene-2-carboxylic acid, monohydrate.

Mefoxin (cefoxitin) is commercially available from Merck and is sodium (6R, 7S)-3-(hydroxymethyl)-7-methoxy-8-oxo-7-[2-(2-thienyl) acetamido]-5-thia-1-azabicylo-[4.2.0]-oct-2-ene-2-carboxylate carbamate (ester).

Merrem (meropenem) is commercially available from Zeneca and is (4R, 5S, 6S)-3-[(3S, 5S)-5-(Dimethylcarbamoyl)-3-pyrrolidinyl] thiol]-6-[(1R)-1-hydroxyethyl]-4-methyl-7-oxo-1-azabicyclo-[3.2.0]-hept-2-ene-2-carboxylic acid trihydrate.

Primaxin (imipenem and cilastatin for injectable suspension) is commercially available from Merck and is (1) imipenem is N-formimidoylthienamycin monohydrate, chemical name is [5R-[5α,6α(R*)]]-6-(1-hydroxyethyl)-3-[[2-[(iminomethyl)amino]ethyl]-thio]-7-oxo-1-azabicylco-[3.2.0]-hept-2-ene-2-carboxylic acid monohydrate, cilastatin sodium is [R-[R*,S*,-(Z)]]-7-[(2-amino-2-carboxyethyl)thio]-2-[[(2,2-dimethyl cyclopropyl)carbonyl]amino]-2-heptenoic acid, monosodium salt.

Ancef (cefazolin) is commercially available from SmithKline Beecham and is 3-{[(5 -methyl-1,3,4-thiadiazol-2-yl)thiomethyl)]}-8-oxo-7-[2-( 1 H-30-tetrazol-1-yl)acetamido]-5-thia-1-azabicyclo [4.2.0] oct-2-ene-2-carboxylic acid.

Ceclor (cefaclor) is commercially available from Lilly and is 3-chloro-7-D-(2-phenylglycinamido)-3-cephem-4-carboxylic acid monohydrate;

Cedax (ceftibuten) is commercially available from Schering and is (+)-(6R,7R)-7-[(Z)-2-(2-(2-amino-4-thiazoly)-4-carboxycrotonamido]-8-oxo-5-thia-1 -azabicyclo-[4.2.0]-oct-2-ene-2-carboxylic acid, dihydrate.

Cefizox (ceftizoxime sodium) is commercially available from Fujisawa and is sodium salt of [6R-[6α7β(Z)]]-7 [[2, 3, dihydro-2-imino-4-thiazolyl) (methoxy amino) acetyl]amino]-8-oxo-5-thia-1-azabicyclo [4.2.0] oct-2-ene-2-carboxyolic acid.

Cefobid (cefoperazone sodium) is commercially available from Pfizer and is sodium (6R,7R)-7-[R-2-(4-ethyl-2,3 -dioxo-1-piperazine-carboxamido)-2-(p-hydroxyphenyl)-acetamido)-3-[[(1-methyl-1H-tetrazol-5-yl)thio]methyl]-8-oxo-5-thia-1-azabicyclo[4.2.0]oct-2-ene-2-carboxylate.

Ceftin (cefuroxime axetil) is commercially available from Glaxo Wellcome and is (R,S)-1-hydroxyethyl(6R, 7R)-7-[2-(2-fWryl)glyoxylamido]-3-(hydroxyethyl)-(8-oxo-5-thia-1-aza-bicyclo[4.2.0]oct-2-ene-2-carboxylate, 7² (Z)-O-methyl-oxime), 1-acetate 3-carbamnate.

Cefzil (cefrozil) is commercially available from Bristol-Myers Squibb and is (6R,7R)-7-[R-2-amino-2-(p-hydroxyphenyl)acetamido3-8-oxo-3-propenyl-5-thia-1-azabicyclo[4.2.0]oct-2-ene-2-carboxylic acid monohydrate.

Ceptaz (ceftazidime) is commercially available from Glaxo Wellcome and is [6R-[6α713 (Z)]]-1-[[7-[[(2-amino-4-thiazolyl)[(1-carboxy-1-methylethoxy)imine]acetyl]amino]-2-carboxy-8-oxo-5-thia-1-azabicyclo[4.2.0]oct-2-en-3-yl]methyl]hydroxide, inner salt.

Claforan (cefotaxime) is commercially available from Hoescht Marion Roussel and is 7-[2-(2-amino-4-thiazolyl)glyoxylamido]-3-(hydroxymethyl)-8-oxo-5-thia-1-azabicyclo-[4.2.0]-oct-2-ene-2-carboxylate 7² (Z)-(O-methyloxime), acetate (ester).

Duricef (cefadroxil monohydrate) is commercially available from Bristol-Myers Squibb and is [6R-[6α, 7β(R*)]]-7-[[amino (4-hydroxyphenyl) acetyl]amino]-3-methyl-8-oxo-5-Thia-1-azabicyclo[4.2.0]oct-2-ene-2-carboxylic acid, monohydrate.

Fortaz (ceftazidime) is commercially available from Glaxo Wellcome and is [6R-[6α, 7β(Z)]]-1 -[[7-[[(2-amino-4-thiazolyl)[1 -carboxy-1-methylethoxy)imino]acetyl]amino]-2-carboxy-8-oxo-5-thia-1-azabicyclo [4.2.0] oct-2-en-3-yl]methyl]-hydroxide, inner salt.

Keflex (cephalexin) is commercially available from Dista and is 7-(D-α-Amino-α-phenyl acetamido)-3-methyl-3-cephem-4-carboxylic acid monohydrate.

Keftab (cephalexin HCl) is commercially available from Dura and is 7-(D-2-Amino-2-phenylacetamido)-3-methyl-3-cephem-4-carboxylic acid hydrochloride monohydrate.

Kefurox (cefuroxime) is commercially available from Lilly and is the sodium salt of (6R. 7R)3 -carbamoyloxymethyl-7-[Z-2 -methoxyimino-2-(fur-2-yl)acetamido]ceph-3 -em-4-carboxylate.

Kefzol (cefazolin) is commercially available from Lilly and is the sodium salt of 3-{[(5-methyl- 1,3,4-thiadiazol-2-yl)thio]methyl}-8-oxo-7-[2-(1H-tetrazol-1-yl)acetamido]-5-thia-1-aza-bicyclo[4.2.0]oct-2-ene-2-caboxylic acid.

Mandol (cefamandole narate) is commercially available from Lilly and is [6R-[6α-7β(R*)]]-7-[[(formyloxy)phenylacetyl]amino]-3-[[(1-methyl-1H-tetrazol-5-yl)thio]methyl]-8-oxo-5-thia-1-azabicyclo[4.2.0]oct-2-ene-2-carboxylic acid, mono-sodium salt.

Maxipime (cefepime HCl) is commercially available from Bristol-Myers Squibb and is 1-[[6R,7R)-7-[2-amino-4-thiazolyl)-glyoxyl1do]-2-carboxy-8-oxo-5-thia-1-azabicyclo-[4.2.0]-oct-2-en-3-yl]methyl]-1-methylpyrrolidinium chloride, 7²-(Z)-(O-methyloxime), monohydrochloride, monohydrate.

Monocid (cefonicid sodium) is commercially available from SmithKline Beecham and is [6R-[6α,7β(R*)]]-[(hydroxyphenyl-acetyl)-amino]-8-oxo-3-[[1-(sulfomethyl)-1H-tetrazol-5-yl] 30 thio-methyl]-5-Thia-1-azabicyclo [4.2.0] oct-2-ene-2-carboxylic acid, disodium salt,.

Omnicef (cefdinir) is commercially available from Parke Davis and is [6R-[6α,7β(Z)]]-7-[[(2-amino-4-thiazolyl)(hydroxyimino)acetyl]amino]-3-ethenyl-8-oxo-5-thia-1-azabicyclo[4.2.0]-oct-2-ene-2-carboxylic acid.

Rocephin (ceftriaxone) is commercially available from Roche Laboratories and is (6R,7R)-7-[2-(2-Amino-4-thiazolyl)glyoxylamido]-8-oxo-3-[[(1,2,5,6-tetrahydro-2-methyl-5,6-di-oxo-as-triazin-3-yl) thio] methyl]-5-thia-1-azabicyclo-[4.2.0]-oct-2-ene-2-carboxylic acid, 7²-(Z)-O-methyloxime), disodium salt, sesquaterhydrate.

Suprax (ceftixime) is commercially available from Lederle Laboratories and is (6R, 7R)-7-[2-(2-amino-4-thiazolyl)glyoxylamido]-8-oxo-3-vinyl-5-thia-1-azabicyclo-[4.2.0]-oct-2-ene-2-carboxylic acid, 7²-(Z)-[O-(carboxymethyl)oxime]trihydrate.

Tazicef (ceftazidime) is commercially available from SmithKline Beecham and is a pyridinium, [6R,[6α,7β(Z)]]-1-[[7-[[2-amino-4-thiazolyl)[(1-carboxy-1-methylethoxy)-imino]acetyl]amino]-2-carboxy-8-oxo-5-thia-1-azabicyclo-(4.2.0)-oct-2-en-3-yl]methyl]-hydroxide, inner salt,.

Tazidime (cefiazidime) is commercially available from Lilly and is pentahydrate of Pyridinium, 1-[[7-[[2-amino-4-thiazolyl)[(1-carboxy-1-methylethoxy)imino]acetyl]amino]-2-carboxy-8-oxo-5-thia-1-azabicyclo(4.2.0)oct-2-en-3-yl]methyl]hydroxide, inner salt, [6R,[6α, 7β(Z)]].

Vantin (cefpodoxime proxetil) is commercially available from Pharmacia & Upjohn and is (RS)-I-(isoproproxycarbonyloxy)ethyl-(+)-(6R,7R)-7-[2-(2-amino-4-thiazolyl)-2-{(Z)-methoxyimino}acetamido]-3-methoxymethyl-8-oxo-5-thia-1-azabicyclo[4.2.0]oct-2-ene-2-carboxylate.

Zinacef (cefuroxime) is commercially available from Glaxo Wellcome and is (6R,7R)-3 -carbamoyloxymethyl-7-[Z-2-methoxy-imino-2-fr-2-yl)-acetamido]-ceph-3-em-4-carboxylate sodium salt.

Biaxin (clarithromycin) is commercially available from Abbott and is 6-O-methylerythromycin.

Dynabac (dirithromycin) is commercially available from Sanofi and is (9S)-9-Deox-11-deoxy-9,11-[imino [](1R)-2-(2-methoxyethoxy)-ethylidene]oxy]erythromycin.

E.E.S. 200 (Erythromycin Ethylsuccinate) is commercially available from Abbott and is erythromycin 2′-(ethylsuccinate).

E.E.S. 400 (Erythromycin Ethylsuccinate) is commercially available from Abbott and is erythromycin 2′-(ethylsuccinate).

Ery-Ped 200 (Erythromycin Ethylsuccinate) is commercially available from Abbott and is erythromycin 2′-(ethylsuccinate).

EryPed 400 (Erythromycin Ethylsuccinate) is commercially available from Abbott and is erythromycin 2′-(ethylsuccinate).

Ery-Tab (Erythromycin delayed-release tablets) is commercially available from Abbott and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11, 13-hexamethyl-6-[[3,4,6-trideoxy-3 -(dimethylamino)-13-β-xylo-hex-opyranosyl]oxy]oxacyclotetra-decane-2,10-dione. Erythrocin Stearate (Erythromycin stearate) is commercially available from Abbott and is the stearate salt of (3R*,4S*,5S*,6R*,7R*,9R*,11R*,12R*, -13S*,14R*)-4-[(2,6-dideoxy-3-C-methyl-3-O-methyl-α-L-ribo-hexopyranosy)oxy]-14-ethyl-7,12,13-trihydroxy-3,5,7,9,11,13-hexamethyl-6-[[3,4,6-trideoxy-3-(dimethylamino)-β-D-xylo-hexopyranosyl]oxy]oxacyclotetradecane-2,10-dione.

Ilosone (erythromycin estolate) is commercially available from Dista and is erythromycin 2′-propionate, dodecyl sulfate.

PCE Dispertab (erythromycin particles in tablets) is commercially available from Abbott and is (3R*,4S*,5S*,6R*,7R*,9R*,11R*,12R*,13S*,14R*)-4-[(2, 6-dideoxy-3-C-methyl-3-O-methyl-ot-L-ribo-hexopyranosyl) oxy]- 14-ethyl-7,12,13-trihydroxy-3,5,7,9,11, 13-hexa-methyl-6-[[3,4,6-tiideoxy- 3-(dimethylamino)-β-D-xylo-hexopyranosyl]oxy]oxacyclotetradecane-2,10-dione.

Pediazole (erythromycin ethylsuccinate and sulfisoxazole acetyl for oral suspension) is commercially available from Ross Products and is 2′-ethylsuccinyl ester of erythromycin (erythromycin ethylsuccinate) and N-(3, 4-dimethyl-5-isoxazolyl)-N-sulfanilylacetamide (sulfisoxazole acetyl).

Tao (troleandomycin) is commercially available from Pfizer and is the synthetically derived acetylated ester of oleandomycin.

Zithromax (azithromycin) is commercially available from Pfizer and is (2R,3S,4R,5R,8R,10R,11R,12S,13S, 14R)-13-[(2,6-dideoxy-3-C-methyl-3-O-methyl-a-L-ribo-hexo-pyranosyl)-oxy]-2-ethyl-3,4,10-trihydroxy-3,5,6,8,10,12,14-heptamethyl-11-[[3,4,6-trideoxy-3-(dimethyl-amino)-β-D-xylo-hexopyranosyl]oxy]-1-oxa-6-azacyclopentadecan-15-one.

Erythromycin, which is (3R*,4S*,5S*,6R*,7R*,9R*,11R*,12R*,13S*,14R*)-4-[(2,6-di-deoxy-3 -C-methyl-3-O-methyl-α-L-ribo-hexopyranosyl)oxy]- 14-ethyl-7,12,13-trihydroxy-3,5,7,9,11,13,hexamethyl-6-[[3,4,6-trideoxy-3-(dimethylamino)-O-β-D-xylo-hexopyranosyl]oxy]oxa-cyclotetradecane-2,10-dione.

Cleocin HCl (clindamycin hydrochloride) is commercially available from Pharmacia & Upjohn and is the hydrated hydrochloride salt of clindamycin, asemisynthetic antibiotic produced by a 7 (S)-chloro-substitution of the (7R) hydroxyl group of lincomycin.

Cleocin Phosphate (clindamycin phosphate) is commercially available from Pharmacia & Upjohn and is L-threo-α-D-galacto-Octopyranoside, (2S-trans)-methyl-7-chloro-6,7,8-trideoxy-6-[[(1-methyl-4-propyl-2-pyrrolidinyl)carbonyl]amino]-1-thio-2-(dihydrogen phosphate).

Coly-Mycin M (colistimethate sodium) is commercially available from Monarch.

Vancocin HCl (vancomycin hydrochloride) is commercially available from Lilly.

Amoxil (amoxicillin) is commercially available from SmithKline Beecham and is (2S, 5R,6R)-6-[R-(−)-2-amino-2-(p-hydroxyphenyl)acetamido]-3,3-dimethyl-7-oxo-4-thia-1-aza-bicyclo-[3.2.0]-heptane-2-carboxylic acid trihydrate.

Augmentin (amoxicillin/clavulanate potassium) is commercially available from SmithKline Beecham and is the trihydrate of (2S,5R,6R)-6-[R-(−)-2-amino-2-p-hydroxyphenyl)acetamido]-3,3-dimethyl-7-oxo-4-thia-1-azabicyc-[3.2.0]-heptane-2-carboxylic acid (amoxicillin) and potassium (Z)-(2R,5R)-3-(2-hydroxyethylidene)-7-oxo-4-oxa-1-aza-bicyclo-[3.2.0]-heptane-2-carboxylate (clavulanate potassium).

Bicillin C-R 900/300 (Penicillin G benzathine and Penicillin G procaine suspension) is commercially available from Wyeth-Ayerst and is (2S, 5R,6R)-3,3-Dimethyl-7-oxo-6-(2-phenyl-acetamido)-4-thia-1-azabicyclo-[3.2.0]-heptane-2-carboxylic acid compound with N,N′-dibenzyl-ethylenediamine (2:1), tetrahydrate (Penicillin G benzathine) and (2S,5R,6R)-3,3-Dimethyl-7-oxo-6-(2-phenylacetamido)-4-thia-1-azabicyclo-[3.2.0]-heptane-2-carboxylic acid compound with 2-(diethylamino)ethyl p-amino benzoate compound (1:1) monohydrate (Penicillin G procaine).

Bicillin C-R (Penicillin G benzathine and Penicillin G procaine suspension) is commercially available from Wyeth-Ayerst and is (2S,5R,6R)-3,3-Dimethyl-7-oxo-6-(2-phenyl-acetamido)-4-thia-1-azabicyclo-[3.2.0]-heptane-2-carboxylic acid compound with N,N′-dibenzyl-ethylenediamine (2:1), tetrahydrate (Penicillin G benzathine) and (2S,5R,6R) 3,3-Dimethyl-7-oxo-6-(2-phenylacetamido)-4-thia-1-azabicyclo-[3.2.0]-heptane-2-carboxylic acid compound with 2-(diethylamino)ethyl p-amino benzoate compound (1:1) monohydrate(Penicillin G procaine).

Bicillin L-A (Penicillin G benzathine suspension) is commercially available from Wyeth-Ayerst and is (2S,5R,6R)-3,3-Dimethyl-7-oxo-6-(2-phenylacetamido)-4-thia-1-azabicyclo-[3.2.0]-heptane-2-carboxylic acid compound with N,N′-dibenzylethylene-diamine (2:1), tetrahydrate.

Geocillin (carbencillin indanyl sodium) is commercially available from Pfizer and is 1-(5-Indanyl)-N-(2-carboxy-3-3-dimethyl-7-oxo-4-thia-1-azabicyclo-[3.2.0]-hept-6-yl)-2-phenyl-malonamate monosodium salt.

Mezlin (sterile mezlocillin sodium) is commercially available from Bayer and is the monohydrate sodium salt of 6-{D-213[(methyl-sulfonyl)-2-oxo-imidazolidine-1-carboxamido]-2-phenylacetamido} penicillanic acid.

Omnipen (ampicillin) is commercially available from Wyeth-Ayerst and is (2S,5R,6R)-6-[R-2-Amino-2-phenylacetamido]-3,3 -dimethyl-7-oxo-4-thia-1-aza-bicyclo-[3.2.0]-heptane-2-carboxylic acid.

Pen-Vee K (penicillin V potassium) is commercially available from Wyeth-Ayerst and is the potassium salt of the phenoxymethyl analog of penicillinG.

Pfizerpen (penicillin G potassium) is commercially available from Pfizer and is monopotassium 3,3-dimethyl-7-oxo-6-(2-phenylacetamido)-4-thia-1-azabicyclo-(3.2.0)-heptane-2-carboxylate.

Pipracil (piperacillin sodium) is commercially available from Lederle and is the monosodium salt of [2S-[2α,5α,6β(S*)]]-6-[[[[(4-ethyl-2-3-dioxo-1-piperazinyl)carbonyl]-amino]phenylacetyl]amino]-3,3-di-methyl-7-oxo-4-thia-1-azabicyclo-[3.2.0]-heptane-2-carboxylic acid, monosodium salt.

Spectrobid (bacampicillin HCl) is commercially available from Pfizer and is 1′-ethoxy-carbonyloxyethyl-6-(D-αamninophenylacetamide)penicillate hydrochloride.

Ticar (ticarcillin disodium) is commercially available from SmithKline Beecham and is the disodium salt of N-(2-carboxy-3,3-dimethyl-7-oxo-4-thia-1-azabicyclo-[3.2.0]-hept-6-yl)-3-thiophenemalonamic acid.

Timentin (ticarcillin disodium and clavulanate potassium) is commercially available from SmithKline Beecham and is N-(2-carboxy-3,3-dimethyl-7-oxo-4-thia-1-azabicyclo-[3.2.0]-hept-6-yl)-3-thiophenemalonamnic acid disodium salt (ticarcillin disodium) and potassium (Z)-(2R,5R)-3-(2-hydroxyethylidene)-7-oxo-4-oxa-1-azabicyclo-[3.2.0]-heptane-2-carboxylate (clavulanate potassium).

Unasyn (ampicillin sodium/sulbactam sodium) is commercially available from Pfizer and is monosodium (2S,5R,6R)-6-[R-2-Amino-2-phenyl acetamido]-3,3-dimethyl-7-oxo-4-thia-1-aza-bicyclo-[3.2.0]-heptane-2-carboxylate (amipicillin sodium) and sodium penicillate sulfone; sodium (2S,5R)-3,3-dimethyl-7-oxo-4-thia-1-azabicyclo-[3.2.0]-heptane-2-carboxylate-4,4-di-oxide (sulbactam sodium).

Zosyn (piperacillin sodium and tazobactam sodium) is commercially available from Lederle and is sodium (25,5R,6R)-6[R-2-(4-ethyl-2,3-dioxo-1-piperazine-carboxamido)-2-phenyl-acetamido]-3,3-dimethyl-7-oxo-4-Thia-1-azabicylco-[3.2.0]-heptane-2-carboxylate (piperacillin) and sodium (2S,3S,5R)-3-methyl-7-oxo-3-(1H-1,2,3-triazol-1-ylmethyl)-4-thia-1-azabicyclo-[3.2.0]-heptane-2-carboxylate-4,4-dioxide (tazobactam).

Dicloxacillin Sodium is monosodium (2S,5R,6R)-6-(3-(2,6-dichlorophenyl)5-methyl-4-isoxazolecarboxamido]-3,3-dimethyl-7-OXO-4-thia-1-azabicyclo-[3.2.0]-heptane-2-carboxylate monohydrate.

Achromycin V (tetracycline HCl) is commercially available from Lederic and is the monohydrochloride of [4S-(4α,4aα,5aα,6β,12aα,)]-4-(dimethylamino)-1,4,4a,5,5a,6,11,12a-octa-hydro-3,6,10,12,12a-pentahydroxy-6-methyl-1,11-dioxo-2-naphthacenecarboxamide.

Declomycin (demeclocycline HCl) is commercially available from Lederle Laboratories and is 7-chloro-4-dimethylamino-1,4,4,4a,5,5a,6,11,12a-octahydro-3,6,10,12, 12a-pentahydroxy- 1,11 -dioxo-2-naphthaeenecarboxamide monohydrochloride.

Dynacin (minocylcine HCl) is commercially available from Medicis and is [4S-(4α,4aα, 5aα,12aα)]-4,7-bis (dimethylamino)-1,4,4a,5,5a,6,11,12a-octahydro-3,10,12,12a-tetrahydroxy-1,11-dioxo-2-napthacene carboxamide monochloride.

Minocin (minocycline hydrochloride) is commercially available from Lederle Laboratories and is [4S-(4a,4aα,5aα,12aα)]-4,7-bis (dimethylamino)-1,4,4a,5,5a,6,11,12a-octa-hydro-3,10,12,12a-tetrahydroxy-1,11 -dioxo-2-napthacene carboxamide monochloride.

Monodox (Doxycycline monohydrate capsules) is commercially available from Oclassen and is α-6-deoxy-5-oxytetracycline.

Terramycin (oxytetracyline) is commercially available from Pfizer.

Vectrin (minocycline hydrochloride) is commercially available from Warner Chilcott Professional Products and is the monochloride of [4S-(4α,4aα,5aα,12ax)]-4,7-bis-(dimethylamino)-1,4,4a,5,5a,6,11,12a-octahydro-3,10,12,12a-tetrahydroxy- 1,11 -dioxo-2-napthacenecarboxamide monochloride.

Vibramycin Calcium (doxycycline sodium) is commercially available from Pfizer and is the monohydrate of 4-(Dimethylamnino)-1,4,4a,5,Sa,6,11,12a-octahydro-3,5,10,12,12a-pentahydroxy-6-methyl-1,11-dioxo-2-napthacene-carboxamide.

Vibramycin Hyclate (doxycycline hyclate) is commercially available from Pfizer and is α-6-deoxy-5-oxytetracycline.

Vibramycin Monohydrate (doxycycline monohydrate) is commercially available from Pfizer and is 4-(Dimethylamino)-1,4,4a,5,5a,6,11,12a-octahydro-3,5,10,12,12a-pentahydroxy-6-methyl-1,11-dioxo-2-napthacene-carboxamide monohydrate.

Vibra-Tabs (doxycycline hydrate) is commercially available from Pfizer and is a-6-deoxy-5-oxytetracycline.

Vibramycin (doxycycline) is commercially available from Pfizer and is 4-(Dimethylamino)-1,4,4a,5,5a,6,11,12a-octahydro-3,5,10,12,12a-pentahydroxy-6-methyl-1,11-di-oxo-2-napthacene-carboxamide monohydrate.

Lincomycins is monosodium (2S,5R,6R),6-(3-(2,6-dichlorophenyl)5-methyl-4-isoxazole-carboxamido]-3,3-dimethyl-7-oxo-4-thia-1-azabicyclo-[3.2.0]-heptane-2-carboxylate monohydrate.

Cleocin HCl (clindamycin HCl) is commercially available from Pharmacia & Upjohn and is the monohydrochloride of methyl 7-chloro-6,7,8-trideoxy-6-(1-methyl-trans-4-propyl-L-2-pyrrolidinecarboxamido)-1-thio-L-threo-α-D-galacto-octopyranoside.

Abelcet (amphotericin B lipid complex) is commercially available from Libosome Company, Inc. and is [1R-(1R*,3S*,5R*,6R*,9R*,11R*,15S*,16R*,17R*,18S*,19E,21E, 23E,25E,27E,29E,31E,33R*,35S*,36R*,37S*)]-33-[(3-amino-3,6-dideoxy-β-D-manno-pyranosyl)oxy]-1,3,5,6,9,11,17,37-octahydroxy-15,16,18-trimethyl-13-oxo-14,39-dioxabicyclo-[33.3.1]-nonatriaconta- 19,21,23,25,27,29,31 -heptaene-36-carboxylic acid.

AmBisome (amphotericin B) is commercially available from Fujisawa Healthcare and is [1R-(1R*,3S*,5R*,6R*,9R*,11R*,15S*,16R*,17R*,18S*,19E,21E,23E,25E,27E, 29E,31E,33R*,35S*,36R*,37S*)]-33-[(3-amino-3,6-dideoxy-β-D-mannopyranosyl)-oxy]-1,3,5,6,9,11,17,37-octahydroxy-15,16,18-trimethyl-13-oxo-14,39-dioxabicyclo-[33.3.1]-nonatriaconta-19,21,23,25,27,29,31-heptaene-36-carboxylic acid.

Amphotee (amphotericin B cholesterol sulfate complex) is commercially available from Sequus Pharmaceuticals, Inc. and is [1R-(1R*,3S*,5R*,6R*,9R*,11R*,15S*,16R*, 17R*,18S*,19E,21E,23E,25E,27E,29E,3 1E,33R*,35S*,36R*,37S*)]-33-[(3-amino-3,6-dideoxy-β-D-manno-pyranosyl)-oxy]-1,3,5,6,9,11,17,37-oetahydroxy-15,16,18-trimethyl-13-oxo-14,39-dioxabicyclo-[33.3.1]-nonatriaconta-19,21,23,25,27,29,31-heptaene-36-carboxylic acid.

Ancobon (flucytosine) is commercially available from ICN Pharmaceuticals and is 5-fluorocytosine.

Diflucan (fluconazole) is commercially available from Pfizer Inc. and is 2,4-difluoro-α-α′-bis(1H-1, 2, 4-triazol-1-ylmethyl)benzyl alcohol.

Fulvicin P/G (ultramicrosize griseofulvin) is commercially available from Schering.

Fulvicin P/G 165 and 330 (ultramicrosize griseofulvin) is commercially available from Schering.

Grifulvin V (griseoffilvin) is commercially available from Ortho Dermatological.

Gris-PEG (griseoffilvin ultramicrosize) is commercially available from Allergan.

Lamisil (terbinafine hydrochloride) is commercially available from Novartis and is (E)-N-(6,6-dimethyl-2-hepten-4-ynyl)-N-methyl-1-naphthalenemethanamine hydrochloride.

Nizoral (ketoconazole) is commercially available from Janssen and is cis-1-acetyl-4-[4-[[2-(2,4-di-chlorophenyl)-2-(1H-imidazol-1-ylmethyl)-1,3-dioxolan-4-yl]methoxy]-phenyl]piperazine.

Amphotericin B is [1R-(1R*,3S*,5R*,6R*,9R*,11R*,15S*,16R*,17R*,18S*,19E, 21E,23E,25E,27E,29E,31E,33R*,35S*,36R*,37S*)]-33-[(3-amino-3,6-dideoxy-β-D-mannopyranosyl)-oxy]-1,3,5,6,9,11,17,37-octahydroxy- 15,16,18otrimethyl-13-oxo-14,39-dioxabicyclo-[33.3.1]-nonatriaconta-19,21,23,25,27,29,31 -heptaene-36-carboxylic acid.

Lotrimin (clotrimazole) is commercially available from Schering and is 1-(O-chloro-α,α-diphenylbenzyl)imidazole.

Dapsone tablets (dapsone) is commercially available from Jacobus and is 4,4′-diaminodi-phenylsulfone (DDS).

Diflucan (fluconazole) is commercially available from Pfizer and is 2, 4-difiuoro-α-α′-bis(1H-1,2,4-triazol-1-yhnethyl)benzyl alcohol.

Monistat-Derm cream (miconazole) is commercially available from Ortho Dermatological and is 1-[2,4-dichloro-β-{(2,4-dichlorobenzyl)oxy}phenethyl]imidazole mononitrate.

Mycostatin Cream (nystatin) is commercially available from Westwood-Squibb.

Sporanox (itraconazole) is commercially available from Janssen Pharmaceutical and is (±)-1-[(R*)-sec-butyl]-4-[p-[[2R*,4S*)-2-(2,4-dichlorophenyl)-2-(1H-1,2,4-triazol-1 -yl-methyl)-1,3-dioxolan-4-yl]methoxy]phenyl]-1-piperazinyl]phenyl]-Δ²- 1,2,4,triazolin-5-one mixture with (±)-1-[(R*)-sec-butyl]-4-[p-[[2R*,4S*)-2-(2,4-dichlorophenyl)-2-(1H-1,2,4-triazol-1-ylmethyl)-1,3-dioxolan-4-yl]methoxy]phenyl]-1-piperazinyl]phenyl]-Δ²-1,2,4, triazolin-5-one or (±)-1-[(RS)-sec-butyl]-4-[p-[4-[p-[[(2R,4S)-2-(2,4-dichlorophenyl)-2-(1H-1,2,3,4-triazol-1-ylmethy)- 1,3-dioxolan-4-yl]methoxy]phenyl]-1-piperazinyl]phenyl]-Δ²-1,2,4-triazolin-5-one.

Aralen hydrochloride (chloroquine) is commercially available from Sanofi Pharmaceuticals and is the dihydrochloride of 7-(chloro-4-[[4-diethylamino)-1-methylbutyl]-amino]quinoline.

Aralen phosphate (chloroquine phosphate) is commercially available from Sanofi Pharmaceuticals and is 7-(chloro-4-[[4-diethylamino)-1-methylbutyl]amino]-quinoline phosphate (1:2).

Daraprim (pyrimethamine) is commercially available from Glaxo Wellcome and is 5-(4-chlorophenyl)-6-ethyl-2,4-pyrimidinediamine.

Lariam (mefloquine HCl) is commercially available from Roche Laboratories and is (R*, S*)-(±)-α-2-piperidinyl-2, 8-bis(trifluoromethyl)-4-quinoline methanol hydrochloride.

Plaquenil (hydroxychloroquine sulfate) is commercially available from Sanofi Pharmaceuticals and is 2-[[4-[7-chloro-4-quinolyl)amino]pentyl]ethylamino]ethanol sulfate (1:1).

Capastat sulfate (capreomyein sulfate) is commercially available from Dura Pharmaceuticals.

Myambutol (ethambutol hydrochloride) is commercially available from Lederle Laboratories.

Mycobutin (rifabutin capsules) is commercially available from Pharmacia & Upjohn and is 1′,4-didehydro-1-deoxy-1,4-dihydro-5′-(2-methylpropyl)-1-oxorifamycin XIV or (9S,12E,14S,15R,16S,17R,18R,19R,20S,21S,22E,24Z)-6,16,18,20-tetrahydroxy-1-1′-isobutyl-14-methoxy-7,9,15,17,19,21,25-heptamethyl-spiro[9,4-(epoxypentadeca-1,11,13-trienimino)-2H-furo-2′,3′:7,8-naphth-[1,2-d]-imidazole-2,4′-piperidine]-5,10,26-(3H,9H)-trione-16-acetate.

Nydrazid (isoniazid injection) is commercially available from Apothecon.

Paser (aminosalicylic acid) is commercially available from Jacobus and is 4-amino-2-hydroxybenzoic acid.

Priftin (rifapentine) is commercially available from Hoechst Marion Roussel and is rifamycin 3-[[(4-cyclo-pentyl-1-piperazinyl)imino]methyl] or 3[N-(4-cyclopentyl-1-piperazinyl)-formimyidoyl]-2,7-(epoxypentadeca[1,11,13]trienimino)naphtha[2,1-b]furan-1,11(2H)-dione-21-acetate.

Pyrazinamide tablets (pyrazinamide) is commercially available from Lederle Laboratories and is the pyrazine analogue of nicotinamide.

Rifadin (rifampin capsules) is commercially available from Hoechst Marion Roussel and is 3-[[(4-methyl-1-piperazinyl)imino]methyl] rifamycin or 5,6,9,17,19,21-hexahydroxy-23-methoxy-2,4,12,16,20,22-heptamethyl-8-[N-methyl-1-piperazinyl)formimidoyl]-2,7-(epoxypentadeca-[1,11,13]-trienimino)naptho[2,1-b]furan-1,11 (2H)-dione 21-acetate.

Rifadin IV (rifampin for injection) is commercially available from Hoechst Marion Roussel and is 3-[[3-(4-methyl-1-piperazinyl) formnimidoyl]-2,7-(epoxypentadeca[1,11,13]-trienimino)naphtho [2,1-b]furan-1,11-(2H)-dione-21 -acetate.

Rifamate (rifampin and isoniazid) is commercially available from Hoechst Marion Roussel and is 3-(4-methyl-1-piperazinyliminomethyl) rifamycin SV (rifampin) and hydrazide of isonicotinic acid (isoniazid).

Rifater (rifampin, isoniazid and pyrazinamide) is commercially available from Hoechst Marion Roussel and is 3-(4-methyl-1-piperazinyliminomethyl) rifamycin SV (rifampin), hydrazide of isonicotinic acid (isoniazid) and pyrazine analogue of nicotinamide (pyrazinamide).

Seromycin (cycloserine capsules) is commercially available from Dura Pharmaceuticals and is 3-isoxazolidinone, 4-amino-, R—.

Streptomycin Sulfate is commercially available from Pfizer and is O-2-deoxy-2-(methylamino)-α-L-glucopyranosyl-(1→2)-O-5-deoxy-3-C-formyl-α-L-lyxofuranosyl-(1→4)-N-N′-bis(aminoiminomethyl)-, sulfate (2:3) salt.

Tice BCG (BCG vaccine) is commercially available from Organon and is attenuated live Mycobacterium bonis strains Bacillus of Calmette and Guerin.

Cycloserine (seromycin capsules) is commercially available from Dura Pharmaceuticals and is R-4-amino-3-isoxazolidinone.

Nydrazid (Isoniazid) is commercially available from Apothecon and is the hydrazide of isonicotinic acid.

Urised (Methenamine) is commercially available from Poly Medica.

Trecator-SC (ethionamide tablets) is commercially available from Wyeth-Ayerst and is 2-ethylthioisoniocotinamide.

Alferon N (interferon alfa-n3) is commercially available from Interferon Sciences and is interferon alfa-n3 (human leukocyte derived).

Crixivan (indinavir sulfate) is commercially available from Merck & Co., Inc. and is [1(1 S,2R),5(S)]-2,3,5-trideoxy-N-(2,3-dihydro-2-hydroxy-1H-inden-1 -yl)-5-[2-[[1,1-dimethylethyl)amino]carbonyl]-4-(3-pyridinyl-methyl)-1-piperazinyl]-2-(phenylmethyl)-D-erythropentonamide sulfate (1:1).

Cytovene (ganciclovir) is commercially available from Roche and is 9-[[2-hydroxy-1-(hydroxymethyl)ethoxy]methyl]guanine.

Cytovene-IV (ganciclovir sodium) is commercially available from Roche and is 9-[[2-hydroxy-1-(hydroxymethyl) ethoxy]methyl]guanine.

Epivir (lamivudine) is commercially available from Glaxo Wellcome and is (2R,cis)-4-amino-1-(2-hydroxymethyl-1,3-oxathiolan-5-yl)-1H)-pyrimidin-2-one.

Famvir (famciclovir) is commercially available from SmithKline Beecham and is 2-[2-(2-amino-9H-purin-9-yl) ethyl]-1,3-propanediol diacetate.

Flumadine (rimantadine HCl) is commercially available from Forest and is alpha-methyltricyclo-[3.3.1.1/3.7] decane-1-methanamine hydrochloride.

Foscavir (foscarnet sodium) is commercially available from Astra and is phosphonoformic acid, trisodium salt.

Hivid (zalcitabine) is commercially available from Roche and is 4-amino-1-β-D-2′,3′-dideoxyribofuranosyl-2-(1H)-pyrimidone or 2′, 3′, dideoxyribofuranosyl-2-(1H)-pyrimidone or 2′, 3′-dideoxycytidine.

Intron A (interferon alfa-2b) is commercially available from Schering.

Invirase (saquinavir mesylate) is commercially available from Roche Labs and is N-tert-butyl-decahydro-2-[2R-hydroxy-4-phenyl-3(S)-[[N-(2-quinolylcarbonyl)-L-asparaginyl]amino]butyl-(4aS, 8aS)-isoquinoline-3(S)-carboxamide methanesulfonate.

Norvir (ritonavir) is commercially available from Abbott and is [5S-(5R*,8R*,10R*, 11R*)]-10-Hydroxy-2-methyl-5-(1-methylethyl)-1-[2-(1 -methylethyl)-4-thiazolyl]-3,6-di-oxo-8,11-bis(phenyl-methyl)-2,4,7,12-tetraazatridencan-B-oic acid, 5-thiazolylmethyl ester.

Rebetron combination therapy, which contains Rebetrol (ribavirin which is 1-β-D-ribofuranosyl-1H-1,2,4-triazole-3-carboxamide) and Intron A (inteferon alfa-2b), is commercially available from Schering.

Rescriptor (delavirdine mesylate) is commercially available from Pharmacia & Upjohn and is piperazine, 1-[3-[(1-methylethyl)amino]-2-pyridinyl]-4-[[5(methylsulfonyl)-amino]-1H-indol-2-yl] carbonyl], monomethanesulfonate.

Retrovir (ziduvudine) is commercially available from Glaxo Wellcome and is 3′-azido-3′-deoxythymidine.

Retrovir IV (ziduvudine) is commercially available from Glaxo-Wellcome and is 3′-azido-3′-deoxythymidine.

Symmetrel (amantadine hydrochloride) is commercially available from MedImmune Inc. and is humanized monoclonal antibody (IgGlₓ).

Valtrex (valacyclovir HCl) is commercially available from Glaxo Wellcome and is L-valine, 2-[(2-amino-1,6-dihydro-6-oxo-9H-purin-9-yl)methoxy]ethyl ester, monohydrochloride.

Videx (didanosine) is commercially available from Bristol-Myers Squibb Oncology/Immunology and is 2′,3′-di-deoxyinosine.

Viracept (nelfinavir mesylate) is commercially available from Agouron and is [3S-[2(2S*,3S*),3α,4aβ,8aβ]]-N-(1,1-dimethylethyl)decahydro-2-[2-hydroxy-3-[3-hydroxy-2-methyl-benzoyl)amino]-4-(phenylthio)butyl]-3-isoquinolinecarboxcamide monomethanesulfonate (salt).

Viramune (nevirapine) is commercially available from Roxane and is 11-cyclopropyl-5,11 -dihydro-4-methyl-6H-dipyrido [3,2-b:2′,3′-][1,4]diazepin-6-one.

Virazole (ribavirin) is commercially available from ICN and is 1-beta-D-ribofuranosyl-1H-1,2,4-triazole-3-carboxamide.

Vistide (cidofovir) is commercially available from Gilead Sciences and is 1-[(S)-3-hydroxy-2-(phosphonomethoxy)propyl]cytosine dihydrate (HPMPC).

Zerit (stavudine (d4T)) is commercially available from Bristol-Myers Squibb Oncology/Immunology and is 2′,3′-didehydro-3′deoxythymidine.

Symmetrel Syrup (amantadine HCl) is commercially available from Endo Labs and is 1-adamantanarnine hydrochloride.

Combivir Tablets (lamiduvine) is commercially available from Glaxo Wellcome and is 2′,3′-didehydro-3′-deoxythymidine.

Zovirax (acyclovir) is commercially available from Glaxo Wellcome and is 2-amino-1,9-dehydro-9-[(2-hydroxyethyoxy)methyl]-6H-purin-6-one.

Dapsone Tablets (dapsone) is commercially available from Jacobus and is 4,4′-diaminodiphenylsulfone (DDS).

Daraprim (pyrimethamine) is commercially available from Glaxo Wellcome and is 5-(4-chlorophenyl)-6-ethyl-2,4-pyrimidinediamine.

Flagyl 375 (metronidazole) is commercially available from Searle and is 2-Methyl-5-nitro-imidazole-1-ethanol.

Flagyl ER Tablets (metronidazole) is commercially available from Searle and is 2-Methyl-5-nitro-imidazole-1-ethanol.

Flagyl I.V. (metronidazole) is commercially available from SCS and is 2-Methyl-5-nitro-imidazole-1-ethanol.

Furoxone (furazolidone) is commercially available from Roberts and is 3-(5-nitrofiurfuryliden-amino)-2-oxazolidinone.

Mepron (atovaquone) is commercially available from Glaxo Wellcome and is trans-2-[4-(4-chlorophenyl)cyclohexyl]-3-hydroxy-1,4-naphthalenedione.

Neutrexin (trimetrexate glucuronate) is commercially available from U.S. Bioscience and is 2,4-diamino-5-methyl-6-[(3,4,5-trimethoxyanilino)methyl]quinazoline mono-D-glucuronate.

Cipro (ciprofloxacin HCl) is commercially available from Bayer and is the monohydrochloride monohydrate salt of 1-cyclopropyl-6-fluoro-1, 4-dihydro-4-oxo-7-1(1-piperazinyl)-3-quinolinecarboxylic acid.

Floxin (ofloxacin) is commercially available from Ortho-McNeil Pharmaceutical and is (±)-9-fluoro-2,3-dihydro-3-methyl-10-(4-methyl-1-piperazinyl)-7-oxo-7H-pyrido[1,3,3-de]-1,4-benzoxazine-6-carboxylic acid.

Levaquin (levofloxacin) is commercially available from Ortho-McNeil Pharmaceutical) and is (−)-(S)-9-fluoro-2,3-dihydro-3-methyl-10-(4-methyl-1-piperazinyl)-7-oxo-7H-pyrido-[1,2,3-de]-1,4-benzoxazine-6-carboxylic acid hemihydrate.

Mazaquin (lomefloxacin HCl) is commercially available from Unimed and is monohydrochloride salt of (±)-1-ethyl-6,8-difluoro-1,4-dihydro-7-(3-methyl-1-piperazinyl)-4-oxo-3-quinolinecarboxylic acid.

Noroxin (norfloxacin) is commercially available from Merck and is 1-ethyl-6-fluoro-1,4-dihydro-4-oxo-7-(1-piperazinyl)-3-quinolinecarboxylic acid.

Penetrex (enoxacin) is commercially available from Rhône-Poulenc Rorer and is 1-ethyl-6-fluoro-1,4-dihydro-4-oxo-7-(1-piperazinyl)-1,8-naphthyridine-3-carboxylic acid sesquihydrate.

Raxar (grepafloxacin HCl) is commercially available from Glaxo Wellcome and is (±)-1-cyclopropyl-6-fluoro- 1,4-dihydro-5 -methyl-7-(3-methyl-1-piperazinyl)-4-oxo-3-quinolinecarboxylic acid monochloride sesquihydrate.

Trovan (trovafloxacin mesylate) is commercially available from Pfizer and is (1α, 5α,6a)-7-(6-amino-3-azabicyclo[3.1.0]hex-3-yl)- 1 -(2,4-difluorophenyl)-6-fluoro- 1,4-dihydro-4-oxo-1,8-naphthyridine-3-carboxylic acid, monomethanesulfonate.

Zagam (sparfloxacin) is commercially available from Rhône-Poulenc Rorer and is 5-amino-1-cyclopropyl-7-cis-3,5-dimethyl-1-piperazinyl)-6,8-difluoro-1,4,dihydro-4-oxo-3-quinolinecarboxylic acid.

Bactrim (trimethoprim and sulfamethoxazole) is commercially available from Roche Labs and is 2,4-diamino-5-(3,4,5-trimethoxybenzyl) pyrimidine (trimethoprim) and N¹-(5-methyl-3-isoxazolyl)sulfanilamide (sulfamethoxazole).

Bactrim DS (trimethoprim and sulfamethoxazole double strength) is commercially available from Roche Labs and is 2,4-diamino-5-(3,4,5-trimethoxybenzyl)pyrimidine (trimethoprim) and N¹-(5-methyl-3-isoxazolyl)sulfanilamide (sulfamethoxazole).

Pediazole (erythromycin ethylsuccinate and sulfisaxazole acetyl) is commercially available from Ross and is erythromycin 2′-(ethylsuccinate) and N′-acetylsulfisoxazole (sulfisoxizole is N-(3,4-dimethyl-5-isoxazolyl)-N-sulfanilyl acetamide.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Co-trimoxazole is a combined chemotherapeutic agent consisting of trirnethoprim (T) and the sulphonamide sulphamethoxazole (S); their ratio is 1:5. It is bactericidal by virtue of a sequential blockade of the folic acid synthesis in microorganisms. The antimicrobial spectrum of co-trimoxazole includes many Gram-positive and Gram-negative aerobes, Chlamydias, nocardias, protozoa (pneumocystis carinii), etc. In addition to its use for pneumocystis, co-trimoxazole mainly has practical importance against Gram-positive aerobes (urinary tract infections), pneumococci and haemophilus influenza (respiratory tract infections and otitis). http://www.infomed.org/100drugs/ctrifram.html.

Bactrim I.V. Infusion (sulfamethoxazole) is commercially available from Roche Labs.

Pediazole (erythromycin ethylsuccinate and sulfisoxazole acetyl) is commercially available from Ross and is erythromycin 2′-(ethyl succinate) and N′ acetyl sulfisoxazole (sulfisoxizole is N-(3,4-Dimethyl-5-isoxazolyl)-N-sulfanilyl acetamide.

Furadantin (nitrofurantoin) is commercially available from Dura and is 1-[[(5-nitro-2-furanyl)methylene]amino]-2,4-imidazolidinedione.

Macrobid (nitrofurantoin monohydrate macrocrystals) is commercially available from Procter & Gamble Pharmaceuticals and is 1-[[[5-nitro2-furanyl]methylene]amino]-2-4-imidazolidinedione monohydrate.

Macrodantin (nitrofurantoin macrocrystals) is commercially available from Procter & Gamble Pharmaceuticals and is 1-[[[5-nitro-2-furanyl]methylene]amino]-2-4-imidazolidine-dione.

Monurol Sachet (fosfomycin tromethamine) is commercially available from Forest and is (1R, 2S)-(1,2-epoxypropyl) phosphonic acid, compound with 2-amnino-2-(hydroxymethyl)-1,3-propanediol (1:1).

NegGram Caplets (nalidixic acid) is commercially available from Sanofi and is 1-ethyl-1,4-dihydro-7-methyl-4-oxo- 1,8-naphthyridine-3-carboxylic acid.

Septra (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Septra DS (trimethoprim and sulfamethoxazole) is commercially available from Monarch and is 5-[(3,4,5-trimethoxyphenyl)methyl]-2,4-pyrimidinediamine (trimethoprim) and 4-amino-N-(5-methyl-3-isoxazolyl)benzenesulfonamide (sulfamethoxazole).

Urised (a combination of the antiseptics methenamine, methylene blue, phenyl salicylate, benzoic acid and parasympatholytics (atropine sulfate)hyoscyamine) is commercially available from Poly Medica.

Urobiotic-250 Capsules (oxytetracycline HCl, sulfamethizole and phenazopyridine HCl) is commercially available from Pfizer.

Uroqid Acid No. 2 Tablets (methenamine mandelate) is commercially available from Beach.

Bactroban (mupirocin) is commercially available from SmithKline Beecham and is (αE,2S,3R,4R,5S)-5-[(2S,3S,4S,5S)-2,3-Epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Chloromycetin opthalmic (chloramphenical) is commercially available from Monarch and is (1) 2,2-dichloro-N-[2-hydroxy-1-(hydroxymethyl)-2-(4-nitrophenyl) ethyl]acetamide and (2) D-threo-(−)-2,2-Dichloro-N-[β-hydroxy-α-(hydroxymethyl)-p-nitrophenethyl]acetamide.

Cortisporin (neomycin and polymyxin β sulfates and hydrocortisone acetate cream) is commercially available from Monarch and is 21-(acetyloxy)-11β,17-dihydroxypregn-4-ene-3,20-dione.

Ilotycin (erythromycin opthalmic ointment) is commercially available from Dista and is (3R*,4S*,5S*,6R*,7R*,9R*, 11R*,12R*,13S*,14R*)-4-[(2,6-dideoxy-3-C-methyl-3-O-methyl-α-L-ribo-hexopyranosyl)oxy]- 14-ethyl-7,12,13-trihydroxy-3,5,7,9,11,13-hexamethyl-6-[[3,4,6-tri-deoxy-3-(dimethylamino)-β-D-xlo-hexopyranosyl]oxy]oxacyclotetradecane-2,10-dione.

NeoDecadron (neomycin sulfate—dexamethasone sodium phosphate) is commercially available from Merck and is 9-fluoro-11β,17-dihydroxy-16α-methyl-21-(phosphonooxy)pregna-1,4-diene-3,20-dione disodium salt.

Polytrim (trimethoprim and polythyxin β sulfate opthalmic solution) is commercially available from Allergan and is 2,4-diamino-5-(3,4,5-trimethoxylbenzl)pyrimidine (trimethoprim) and the sulfate salt of polymyxin B₁ and B₂ (polythyxin β sulfate).

Terra-Cortril (oxytetracycline HCl and hydrocortisone acetate) is commercially available from Pfizer.

TobraDex (tobramycin and dexamethasone opthalmic suspension and ointment) is commercially available from Alcon and is O-3-Amino-3-deoxy-a-D-glucopyranosyl-(1→4)-ao-[2,6-diamino-2,3,6-trideoxy-a-D-ribo-hexopyranosyl-1(1→6)]-2-deoxy-L-streptamine. Dexa-methasone: Chemical Name: 9-Fluro-11b,17,21-trihydroxy-16a-methylpregna-1,4-diene-3,20-dione.

Vira-A opthalmic ointment, 3% (vidarabine) is commercially available from Monarch and is 9-β-D-arabinofaranosyl-9H-purin-6-amine monohydrate.

Chibroxin (norfloxacin opthalmic solution) is commercially available from Merck and is 1-ethyl-6-fluoro-1,4-dihydro-4-oxo-7-(1-piperazinyl)-3-quinolinecarboxylic acid.

Ciloxan opthalmic solution, (Ciprofloxacin HCl) is commercially available from Alcon and is the monohydro chloride monohydrate salt of 1-cyclopropyl-6-fluoro-1,4-dihydro-4-oxo-7-(1-piperazinyl)-3-quinoline-carboxylic acid.

Ocuflox opthalmic solution (ofloxacin) is commercially available from Allergan and is (±)-9-Fluoro-2,3-dihydro-3-methyl-10-(4-methyl-1-piperazinyl)-7-oxo-7-H-pyrido[1,2,3-de]-1,4-benzoxazine-6-carboxylic acid.

Blephamide opthalmic ointment (sulfacetamide sodium and prednisolone acetate) is commercially available from Allergan and is N-sulfanilyl-acetamide monosodium salt monohydrate (sulfacetamide sodium) and 11β,17,21-trihydroxypreyna-1,4-diene-3,20-dione-21-acetate (prednisolone acetate).

Blephamide opthalmic suspension (sulfacetamide sodium and prednisolone acetate) is commercially available from Allergan and is N-sulfanilyl- acetamide monosodium salt monohydrate (sulfacetamide sodium) and 11β,17,21-trihydroxypreyna-1,4-diene-3,20-dione-21-acetate (prednisolone acetate).

A/T/S (erythromycin) is commercially available from Hoescht Marion Roussel and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-tzi-deoxy-3-(dimethylamino)-β-D-xlo-hexopyranosyl]oxy]oxacyclotetradecane-2,10-dione.

Bactroban (mupirocin) is commercially available from SKB and is (αE,2S,3R,4R,5S)-5-[(2S,3S,4S,5S)-2,3-epoxy-5-hydroxy-4-methylhexyl]tetrahydro-3,4-dihydroxy-β-methyl-2H-pyran-2-crotonic acid, ester with 9-hydroxynonanoic acid, calcium salt (2:1), dihydrate.

Benzamycin (erythromycin-benzoyl peroxide topical gel) is commercially available from Dermik and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]- 14-ethyl-7,12,13-trihydroxy-3,5,7,9,11,13-hexamethyl-6-[[3,4,6-trideoxy-3-(dimethylamino)-β-D-xylo-hexopyranosyl]-oxy]oxacyclotetra-decane-2, 10-dione (erythromycin).

Betadine (povidone-iodine) is commercially available from Purdue Frederick.

Cleocin T (clindamycin phosphate topical solution) is commercially available from Pharmacia & Upjohn and is methyl-7-chloro-6,7,8-trideoxy-6-[[(1-methyl-4-propyl-2-pyrrolidiny)-carbonyl]aamino]-1-thio-(2S-trans)-L-threo-I-D-galacto-octopyranoside-2-(dihydrogen phosphate).

Clindets (clindamycin phosphate pledgets) is commercially available from Stiefel and is methyl-7-chloro-6,7,8-trideoxy-6-(1-methyl-trans-4-propyl-L-2-pyrrolidine-carboxamido)-1-thio-L-threo-α-D-galacto-octopyranoside-2-dihydrogen phosphate.

Emgel (erythromycin) is commercially available from Glaxo Wellcome and is (3R*,4S*,5S*,6R*,7R*,9R*,11R*,12R*, 13S*,14R*)-4-[(2,6-dideoxy-3-C-methyl-3-O-methyl-α-L-ribo-hexopyranosyl)oxy]-14-ethyl-7,12,13-trihydroxy-3,5,7,9, 11,13-hexamethyl-6-[[3,4,6-tri-deoxy-3 -(dimethyl-amino)-β-D-xylo-hexopyranosyl]oxy]oxacyclotetradecane-2,10-dione.

Erycette (erythromycin topical solution) is commercially available from Ortho Dermatological and is (3R*,4S*,5S*,6R*,7R*,9R*,11R*,12R*,13S*, 14R*)-4-[(2,6-di-deoxy-3-C-methyl-3-O-methyl-α-L-ribo-hexopyranosyl)oxy]-14-ethyl-7,12,13-trihydroxy-3,5,7,9,11,13-hexamethyl-6-[[3,4,6-trideoxy-3-(dimethyl-amino)-β-D-xylo-hexopyranosyl]-oxy]oxacyclotetra-decane-2,10-dione.

Klaron (sodium sulfacetamide lotion) is commercially available from Dermik.

Mycostatin (nystatin cream) is commercially available from Westwood-Squibb.

Theramycin Z (erythromycin topical solution) is commercially available from Medicis and is (3R*,4S*,5S*,6R*,7R*,9R*,11R*,12R*,13S*,14R*)-4-[(2,6-dideoxy-3-C-methyl-3-O-methyl-α-L-ribo-hexopyranosyl)oxy]-14-ethyl-7,12,13-trihydroxy-3,5,7,9,11,13,hexamethyl-6-[[3,4,6-trideoxy-3-(dimethylamino)-β-D-xyo-hexopyranosyl]-oxy]oxacyclotetradecane-2,10-dione.

T-Stat (erythromycin) is commercially available from Westwood-Squibb and is (3R*,4S*,5S*,6R*,7R*,9R*,11R*,12R*,13S*,14R*)-4-[(2,6-dideoxy-3-C-methyl-3-O-mehmethyl-α-L-ribo-hexopyranosyl)oxy]-14-ethyl-7,12,13-trihydroxy-3,5,7,9,11,13-hexa-methyl-6-[[3,4,6-tri-deoxy-3-(dimethylamino)-β-D-xylo-hexopyra-nosyl]oxy]oxacyclotetra-decane-2,10-dione.

Exelderm (sulconazole nitrate) is commercially available from Westwood-Squibb and is (±)-1-[2,4-dichloro-β-[(p-chlorobenzyl)-thio]-phenethyl]imidazole mononitrate.

Fungizone (amphotericin B oral suspension) is commercially available from Bristol-Myers Squibb and is [1R-(1R*,3S*,5R*,6R*,9R*,11R*,15S*,16R*,17R*,18S*,19E,21E, 23E,25E,27E,29E,31E,33R*,35S*,36R*,37S*)]-33-[(3-Amino-3,6-dideoxy-β-D-manno-pyranosyl)-oxy]-1,3,5,6,9,11,17,37-octahydroxy-15,16,18-trimethyl-13-oxo-14,39-dioxabi-cyclo-[33.3.1]-nonatriaconta-19,21,23,25,27,29,31-heptaena-36-carboxylic acid.

Lamisil (terbinafine hydrochloride cream) is commercially available from Novartis and is the hydrochloride of (E)-N-(6,6-dimethyl-2-hepten-4-ynyl)-N-methyl-1-naphthalene-methanamine.

Loprox (ciclopiroxolamine) is commercially available from Hoescht Marion Roussel and is 6-cyclohexy-1-hydroxy-4-methyl-2(1H)-pyridone, 2-amino-ethanol salt.

Lotrimin (clotrimazole) is commercially available from Schering and is 1-(O-Chloro-α,α-diphenylbenzyl)imidazole.

Lotrisone (clotrimazole and betamethasone diproprionate) is commercially available from Schering and is 1-(O-Chloro-α,α-diphenyl benzyl)imidazole (clotrimazole) and 9-fluoro-11β,17,21 -trihroxy-16β-methylpregna-1,4-diene-3,20-dione-17,21 -diproprionate (betamethasone diproprionate).

Mentax (butenafme HCl) is commercially available from Penederm and is N-4-tert-butylbenzyl-N-methyl-1-naphthalenemethylamine hydrochloride.

Monistat-Derm (miconazole nitrate) is commercially available from Ortho Dermatological and is 1-[2,4-dichloro-β-{(2,4-dichlorobenzyl)oxy)}phenethyl]imidazole mononitrate.

Mycelex (clotrimazole) is commercially available from Alza and is [1-(O-chloro-α,α-di-phenylbenzyl)imidazole.

Mycostatin (nystatin) is commercially available from Westwood-Squibb.

Naftin (naftifine HCl) is commercially available from Allergan and is (E)-N-cinnamyl-N-methyl-1-naphthalene-methylamine hydrochloride.

Nizoral (ketoconazole) is commercially available from Janssen and is cis-1-acetyl-4[4-[[2-(2,4-dichorophenyl)-2-(1H-imidazol-1-ylmethyl)-1,3-dioxolan-4-yl]methoxy]phenyl]-piperazine.

Nystop (nystatin) is commercially available from Paddock.

Oxistat (oxiconazole nitrate) is commercially available from Glaxo Wellcome and is 2′,4′-dichloro-2-imidazole-1-ylacetophenone-(Z)-[O-(2,4-dichlorobenzyl)oxime mononitrate.

Selsun Rx (2.5% selenium sulfide lotion) is commercially available from Ross.

Spectazole (econazole nitrate) is commercially available from Ortho Dermatological and is 1-[2-{(4-chorophenyl)methoxy}-2-(2,4-dichlorophenyl)-ethyl]-1H-imidazole mono-nitrate.

Denavir (penciclovir cream) is commercially available from SmithKline Beecham and is 9-[4-hydroxy-3-(hydroxymethyl) butyl]guanine.

Zovirax (acyclovir) is commercially available from Glaxo-Wellcome and is 2-amino-1,9-dihydro-9-(2-hydroxyethoxy)methyl-6H-purin-6-one.

Benzashave (benzoyl peroxide) is commercially available from Medicis.

Betadine (povidone-iodine) is commercially available from Purdue Frederick.

Betasept (chlorhexidine gluconate) is commercially available from Purdue Frederick.

Cetaphil (soap substitute) is commercially available from Galaderma.

Clorpactin WCS-90 (sodium oxychlorosene) is commercially available from Guardiam Laboratories.

Dapsone Tablets (dapsone) is commercially available from Jacobus and is 4,4′-diamino-diphenylsulfone (DDS).

Desquam-E (benzoyl peroxide) is commercially available from Westwood-Squibb.

Desquam-X (benzoyl peroxide) is commercially available from Westwood-Squibb.

Hibiclens (chlorhexidine gluconate) is commercially available from Zeneca.

Hibistat (chlorhexidine gluconate) is commercially available from Zeneca.

Inpregon (tetrachlorosalicylanilide 2%) is commercially available from Fleming.

MetroCream (metronidazole) is commercially available from Galaderma and is 2-methyl-5-nitro-1H-imidazole-1-ethanol.

MetroGel (metronidazole) is commercially available from Galaderma and is 2-methyl-5-nitro-1H-imidazole-1-ethanol.

Noritate (metronidazole) is commercially available from Dermik and is 2-methyl-5-nitro-1H-imidazole-1-ethanol.

pHisoHex (hexachlorophene detergent cleanser) is commercially available from Sanofi and is 2,2′-methylene-bis[3,4,6-trichlorophenol].

Sulfacet-R (sodium sulfacetamide 10% and sulfur 5%) is commercially available from Dermik.

Sulfamylon (matenide acetate) is commercially available from Bertek and is α-amino-p-toluenesulfonamide monoacetate.

Triaz (benzoyl peroxide) is commercially available from Medicis.

Vanoxide-HC (benzoyl peroxide hydrocortisone) is commercially available from Dermik and is 11β,17,21-trihydroxypregn-4-ene-3,20-dione (hydrocortisone).

Acticin (permethrin) is commercially available from Penederm and is (±)-3-phenoxy-benzyl-3-(2,2-dichlorovinyl)-2,2-dimethylcyclopropanecarboxylate.

Elimite (permethrin) is commercially available from Allergan and is (±)-3-phenoxy-benzyl-3-(2,2-dichlorovinyl)-2,2-dimethylcyclopropanecarboxylate.

Eurax (crotamiton) is commercially available from Westwood-Squibb and is N-ethyl-N-(o-methylphenyl)-2-butenamide.

Lindane Lotion USP 1% (lindane) is commercially available from Alpharma.

Efudex (fluorouracil) is commercially available from ICN and is 5-flouro-2,4-(1H,3H)-pyrimidinedione.

Fluoroplex (fluorouracil) is commercially available from Allergan and is 5-flouro-2,4-(1H,3H)-pyrimidinedione.

Furadantin Oral Suspension (nitrofurantoin) is commercially available from Dura and is 1-[[5-nitro-2-furanyl)methylene]amino]-2,4-imidazolidine dione.

Zyvox (linezolid) is commercially available from Pharnacia & Upjohn.

The worldwide exploitation of antibiotics to treat infectious diseases has grown dramatically over the last forty years. In 1954, two million pounds of antibiotics were produced in the United States. Today, the figure exceeds 50 million pounds. According to the Centers Disease Control (CDC), humans consume 235 million doses of antibiotics annually.

Widespread misuse or overuse of antibiotics has fostered the spread of antibiotic resistance and has contributed to the development of a serious public health problem. Antibiotic resistance occurs when bacteria that cause infection are not killed by the antibiotics taken to stop the infection. The bacteria survive and continue to multiply, causing more harm. For example, the bacterium Staphlococous aureus is a major cause of hospital acquired infections that, historically, responded satisfactorily to the antibiotic vancomycin. Recently, however, many strains of S. aureus have been found to be resistant to vancomycin. Moreover, the death rate for some communicable diseases such as tuberculosis have started to rise again, in part because of increases in bacterial resistance to antibiotics.

Vitamin B₁₂

For several years after the isolation of vitamin B₁₂ as cyanocobalamin in 1948, it was assumed that cyanocobalamin and possibly hydroxocobalamin, its photolytic breakdown product, occurred in man. Since then it has been recognized that cyanocobalamin is an artifact of the isolation of vitamin B₁₂ and that hydroxocobalamin and the two coenzyme forms, methylcobalamin and adenosylcobalamin, are the naturally occurring forms of the vitamin.

The structure of these various forms is shown in FIG. 1, wherein X is CN, OH, CH₃ or adenosyl, respectively. Hereinafter, the term cobalamin will be used to refer to all of the molecule except the X group. The fundamental ring system without cobalt (Co) or side chains is called corrin and the octadehydrocorrin is called corrole. FIG. 1 is adapted from The Merck Index, Merck & Co. (11th ed.1989), wherein X is above the plane defined by the corrin ring and nucleotide is below the plane of the ring. The corrin ring has attached six amidoalkyl (H₂NC(O)Alk) substituents, at the 2, 3, 7, 8, 13 and 18 positions, which can be designated a-e and g, respectively. See D. L. Anton et al., J. Amer. Chem. Soc., 102, 2215 (1980).

Methylcobalamin serves as the cytoplasmic coenzyme for N⁵-methyltetrahydrofolate: homocysteine methyl transferase (methionine synthase, EC 2.1.1.13), which catalyzes the formation of methionine from homocysteine. Adenosylcobalamin is the mitochondrial coenzyme for methylmalonyl CoA mutase 25 (EC5.4.99.2) which interconverts methylmalonyl CoA and succinyl CoA.

Vitamin B₁₂ is water soluble, has no known toxicity and in excess is excreted by glomerular filtration. Vitamin B₁₂ alone, however, is not effective in treating or preventing infectious diseases (e.g. bacterial infections).

T. M. Houts (U.S. Pat. No. 4,465,775) reported that the components of the radiolabeled mixture of Niswender et al. did not bind with equal affinity to IF. Houts disclosed that radioiodinated derivatives of the pure monocarboxylic (d)-isomer are useful in assays of B₁₂ in which IF is used.

PCT Publication WO 98/08859 discloses bioconjugates (i.e. conjugates containing a bioactive agent and an organocobalt complex in which the bioactive agent is covalently bound directly or indirectly, via a spacer, to the cobalt atom). The organocobalt complex can be cobalamin and the bioactive agent can be a chemotherapeutic agent. However, only one bioactive agent (i.e. chemotherapeutic agent) is attached to the organocobalt complex (i.e. cobalamin) and the attachment is to the cobalt atom (i.e. the 6-position of cobalamin). The bioactive agent is released from the bioconjugate by the cleavage of the weak covalent bond between the bioactive agent and the cobalt atom as a result of normal displacement by cellular nucleophiles or enzymatic action or by application of an external signal (e.g. light, photoexcitation, ultrasound or the presence of a magnetic field).

PCT Publication WO 97/18231 discloses radionuclide labeling of vitamin B₁₂ through the propionamide moieties on naturally occurring vitamin B₁₂. In WO 97/18231, the inventors converted the propionamide moieties at the b-, d- and e- positions of the corrole ring to monocarboxylic acids, through a mild hydrolysis and separated the carboxylic acids by column chromatography. The inventors then attached a bifunctional linking moiety to the carboxylate function through an amide linkage and a chelating agent to the linking moiety again through an amide linkage. The chelating moiety was then used to attach an imaging radionuclide to the vitamin.

U.S. Pat. No. 5,428,023 to Russell-Jones et al. discloses a vitamin B₁₂ conjugate for delivering oral hormone formulations. The hormones are attached to the vitamin B₁₂ through a hydrolyzed propionamide linkage on the vitamin. The patent states that the method is useful for orally administering hormones, bioactive peptides, therapeutic agents, antigens and haptens and lists as therapeutic agents neomycin, salbutamol cloridine, pyrimethamine, penicillin G, methicillin, carbenicillin, pethidine, xylazine, ketamine hydrochloride, mephanesin and iron dextran. U.S. Pat. No. 5,548,064 to Russell-Jones et al. discloses a vitamin B₁₂ conjugate for delivering erythropoietin and granulocyte-colony stimulating factor, using the same approach as the '023 patent.

PCT Publication WO 94/27641 to Russell-Jones et al. discloses a vitamin B₁₂-polymeric linker system for the oral delivery of various active agents. In particular, WO 94/27641 discloses the attachment of various polymeric linkers to the propionamide positions of the vitamin B₁₂ molecule and the attachment of various bioactive agents to the polymeric linker. Exemplary bioactive agents include hormones, bioactive peptides and polypeptides, antitumor agents, antibiotics, antipyretics, analgesics, anti-inflammatories and haemostatic agents. Exemplary polymers include carbohydrates and branched chain amino acid polymers. The linkers used in WO 94/27641 were all extremely large (each having a molecular weight of about 5000 or greater). Moreover, the linkers were of uncertain length, due to the polymerization process by which they were made.

PCT Publication WO 99/65930 to Russell-Jones et al. discloses the attachment of various agents to the 5′OH position on the vitamin B₁₂ ribose ring. The publication indicates that the system can be used to attach polymers, nanoparticles, therapeutic agents, proteins and peptides to the vitamin.

U.S. Pat. No. 5,574,018 to Habberfield et al. discloses conjugates of vitamin B₁₂ in which a therapeutically useful protein is attached to the primary hydroxyl site of the ribose moiety. The patent lists erythropoietin, granulocyte-colony stimulating factor and human intrinsic factor as therapeutically useful proteins and indicates that the conjugates are particularly well adapted for oral administration.

U.S. Pat. No. 5,840,880 to Morgan, Jr. et al discloses vitamin B₁₂ conjugates to which are linked receptor modulating agents, which affect receptor trafficking pathways that govern the cellular uptake and metabolism of vitamin B₁₂. The receptor modulating agents are linked to the vitamin at the b-, d- or e- position.

The development of new drugs is an essential component to strategies designed to reverse the problem of bacterial resistance, particularly in treating infectious diseases (e.g. bacterial infections). Accordingly, there is a need to identify additional compounds to treat infectious diseases (e.g. bacterial infections). The additional compounds can preferably be administered orally.

### SUMMARY OF THE INVENTION

In one embodiment, a compound is provided that includes a transcobalamin- or intrinsic factor-binding agent (also referred to herein as TC- or IF-binding agent) linked to an antibiotic, or an active residue thereof, or its pharmaceutically acceptable salt or prodrug thereof. In one example, the transcobalamin- or intrinsic factor-binding agent is a vitamin B₁₂ carrier that is covalently linked directly or via a spacer group to the antibiotic. In an alternative embodiment, the transcobalamin- or intrinsic factor-binding agent that is covalently linked to the antibiotic has the chemical structure indicated in formula I. The transcobalamin- or intrinsic factor-binding agent can be covalently linked to the antibiotic via conventional chemical processes. It has been discovered that such compounds will localize in or near the infectious disease, allowing efficient therapy.

In another embodiment, infectious diseases are diagnosed and or mapped by the use of a compound that includes a transcobalamin- or intrinsic factor-binding agent linked to a detectable radionuclide (e.g. metallic radioisotope or non-metallic radioisotope) or paramagnetic metal atom, or its pharmaceutically acceptable salt, which will localize in or near an infectious disease. It has been discovered that a compound wherein a TC- or IF-binding agent is linked to a residue of an imaging agent or its pharmaceutically acceptable salt will localize in or near an infectious disease.

In a preferred embodiment, the antibiotic and/or imaging agent and the TC- or IF-binding agent or its pharmaceutically acceptable salt or prodrug thereof, is delivered to the site of unwanted infection in a manner that bypasses or at least does not rely on, the gastrointestinal route of absorption via the vitamin B₁₂ intrinsic factor binding protein. Preferred modes of administration are parenteral, intraperitoneal, intravenous, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, via rectal, vaginal or urethral administration including via suppository, percutaneous, nasal spray, surgical implant, internal surgical paint, infusion pump or via catheter. In one embodiment, the agent and carrier are administered in a slow release formulation such as a direct tissue injection or bolus, implant, microparticle, microsphere, nanoparticle or nanosphere.

In an alternative embodiment, it has been discovered that an agent for the treatment of infection can be highly and effectively absorbed into a site of unwanted infection by direct or indirect attachment to a compound that binds to the intrinsic factor (IF-binding agent), wherein the IF-binding agent and active agent are administered parenterally, for example, using any of the methods listed above.

The TC- or IF-binding agent and the antibiotic or its pharmaceutically acceptable salt or prodrug thereof, can be administered in the course of surgical or medical treatment of the afflicted site. For example, the TC- or IF-binding agent and active agent can be positioned directly at the site of infection during the course of surgery either by painting the formulation (with or without a controlled release matrix) onto the surface of the afflicted area or by depositing a bolus of material in a suitable matrix that is released into the afflicted area over time. In another embodiment, the TC- or IF-binding agent and the active agent are administered directly into the infection via injection or catheter.

In another embodiment, the TC- or IF-binding agent and the active agent is combined with either intrinsic factor or a transcobalamin carrier protein or both and administered parenterally, for example, via intravenous, intramuscular, direct injection or catheter, to the afflicted location.

It is preferred that the TC- or IF-binding agent and the active agent be administered parenterally and not orally to increase the effectiveness of the agent and, in the case of imaging, to decrease the exposure of normal cells to the imaging agent. It is known that the ileal receptor for intrinsic factor-bound cobalamin is present in the gastrointestinal tract in only very small quantities and on oral delivery of vitamin B₁₂ into the alimentary system the ileal receptor can only absorb approximately two micrograms per day of vitamin B₁₂ for systemic delivery. Even assuming a small amount of systemic absorption via passive transport of a large oral dose, this level of administration is insufficient for the treatment of infectious diseases.

The TC-or IF-binding agent and imaging agent useful to image sites of infection in the body, can optionally be joined by means of a di- or multi-valent linking moiety. The linker used to join the TC- or IF-binding agent and the active agent preferably has a single molecular weight and does not exhibit a molecular weight distribution, for example as found in most polymers. The linker can range in size from small to large molecular weight, as long as there is not a distribution of weights in the linker. It is important to strictly control the uniformity of size of the conjugate for predictability of therapeutic performance.

The linkers preferably have a molecular weight below about 2000, more preferably below about 1900 or 1800 and even more preferably below about 1500 or 1000.

Thus, in one embodiment the invention provides an imaging conjugate having a high specificity for infectious cells, comprising (1) a TC- or IF-binding agent and (2) an imaging agent linked directly or through a linker to the TC- or IF-binding agent, wherein the linker has either (i) a unimodal (i.e. single) and defined molecular weight or (ii) a molecular weight less than about 2000 and preferably, below 1900, 1800 or 1500.

In one embodiment, the TC- or IF-binding agent is any moiety that will bind to a transcobalamin receptor and is able to be linked to an antibiotic, and optionally an imaging agent. Methods for the assessment of whether a moiety binds the TC receptor are known and include those described by Pathare, et al., (1996) Bioconjugate Chem. 7, 217-232; and Pathare, et al., Bioconjugate Chem. 8, 161-172. An assay that assesses binding to a mixture of transcobalamin I and II receptors is found in Chaiken, et al, Anal. Biochem. 201, 197 (1992) An unsaturated vitamin B₁₂ binding capacity (UBBC) assay to assess the in vitro binding of the conjugate to the transcobalamin proteins is described by D. A. Collins and H. P. C. Hogenkamp in J. Nuclear Medicine, 1997, 38, 717-723. See also Fairbanks, V. F. Mayo Clinical Proc. 83, Vol 58, 203-204.

In one embodiment the TC- or IF- binding carrier is represented by formula I.

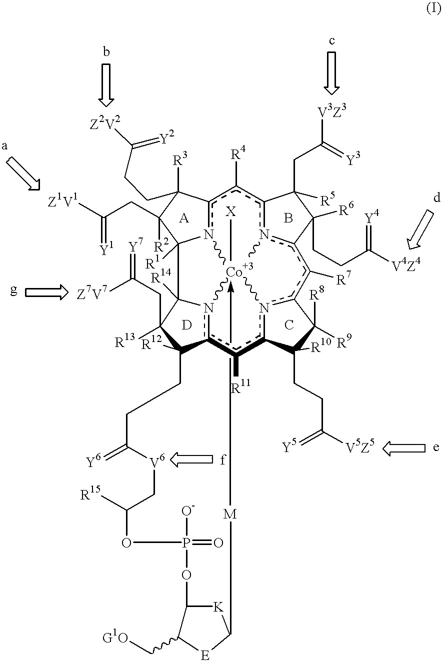

wherein:

the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;

the dotted line in the chemical structure indicates either a double or single bond such that the double bond does not over-extend the valence of the element (i.e. to give pentavalent carbons) and, in the case of a single bond, the valence is completed with hydrogen; and

wherein, in a preferred embodiment, the bonding and stereochemistry of the compound is the same as that of vitamin B₁₂ as it exists in nature.

X is hydrogen, cyano, halogen (Cl, F, Br or I), haloalkyl (including CF₃, CF₂CF₃, CH₂CF₃ and CF₂Cl), NO, NO₂, NO₃, phosphonate (including alkyl-P(O)₂OR¹⁵), PR¹⁵R¹⁶R¹⁷, NH₂, NR¹⁵R¹⁶, OH or ¹⁵, SR¹⁵, SCN, N₃, OC(O)R¹⁵, C(O)₂R¹⁵, C(O)R¹⁵, OC(O)NR¹⁵R¹⁶, C(O)₂NR¹⁵R¹⁶, C(O)NR¹⁵R¹⁶, P(O)₂OR¹⁵, S(O)₂OR¹⁵, a purine or pyrimidine nucleoside or nucleoside analog, including adenosyl (preferably linked through a 5′-deoxy linkage) and 5-FU, alkyl, alkenyl, alkynyl, aryl, aralkyl, alkaryl, amino acid, peptide, protein, carbohydrate, heteroalkyl, heterocycle, heteroaryl or alkylheteroaryl. In one embodiment that is less preferred, X is L-T or L-T′.

M is a monovalent heterocycle or heteroaromatic, which is capable of binding to the adjacent sugar ring. M is preferably a purine or pyrimidine including but not limited to adenine, 2-methyladenine, 2-methylmercaptoadenine, e-methylsulfinyl-adenine, 2-methylsulfonyladenine and guanine; or a benzimidazole, a 5- and/or 6- substituted benzimidazole, such as 5,6-dimethylbenzimidazole, 5-methylbenzimidazole, 5-hydroxy-benzimidazole, 5-methoxy-benzimidazole, naphth-imidazole, 5-hydroxy-6-methyl-benz-imidazole or 5-methoxy-6-methyl-benz-imidazole; or a phenol, such as phenol or p-cresol. The heterocycle or heteroaromatic can optionally be substituted with L-T or L-T′.

K is O, S, NJ¹, C(OH)H, CR¹⁰⁰R¹⁰¹ or C(R¹⁰⁰)V⁸Z⁸.

E is O, S, SO₂ or CH₂.

G¹ is hydrogen, alkyl, acyl, silyl, mono-, di- or tri-phosphate, L-T or L-T′.

Y¹, Y², Y³, Y⁴, Y⁵, Y⁶ and Y⁷ independently are O, S or NJ².

V¹, V², V³, V⁴, V⁵, V⁶, V⁷ and V⁸ independently are O, S, NJ³, CR¹⁰²R¹⁰³ or a direct bond.

Z¹, Z², Z³, Z⁴, Z⁵, Z⁷ and Z⁸ independently are R¹⁰⁴, L-T or L-T′.

Each L is independently a direct bond or a linker to one or more T or T′ moieties and that does not significantly impair the ability of the TC- or IF-binding agent to bind to a transcobalamin receptor.

Each T independently comprises an antibiotic agent, or a pharmaceutically acceptable residue thereof, optionally bound though a chelating moiety if necessary or desired. Each T′ independently comprises an imaging agent, optionally bound though a chelating moiety if necessary or desired. In one embodiment, T is an antibiotic for the treatment or prevention of infection. In an alternate embodiment, T′ is an imaging agent for the diagnosis of infection.

At least one of Z¹, Z², Z³, Z⁴, Z⁵, Z⁷, Z⁸, X, M and G¹ is independently L-T or L-T′. In a preferred embodiment, at least one of Z¹, Z², Z³, Z⁴, Z⁵, Z⁷, Z⁵ and G¹ is independently L-T, wherein T is independently an antibiotic. In another embodiment, the compound of formula I contain at least one T that is independently an antibiotic and at least one T′ that is independently an imaging agent. In a preferred embodiment, Z² comprises the sole L-T in the TC- or IF-binding agent.

J¹, J² and J³ independently are hydrogen, alkyl, alkenyl, alkynyl, alkaryl, cycloalkyl, aryl, cycloaryl, heteroalkyl, heterocycle, heteroaryl, hydroxyl, alkoxy or amine.

R¹, R², R³, R⁴, R⁵, R⁶, R⁷, R⁸, R⁹, R¹⁰, R¹¹, R¹², R¹³ and R¹⁴ independently are hydrogen, lower alkyl, lower alkenyl, lower alkynyl, lower cycloalkyl, heteroalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine.

R¹³ and R¹⁴ optionally can form a double bond.

R¹⁵, R¹⁶ and R¹⁷ are independently hydrogen, alkyl, alkenyl, alkynyl, aryl, alkaryl or aralkyl group, heteroalkyl, heterocycle or heteroaromatic.

R¹⁰⁰, R¹⁰¹, R¹⁰², R¹⁰³ and R¹⁰⁴ are independently hydrogen, alkyl, alkenyl, alkynyl, aryl, acyl, heteroaromatic, heteroaryl, heteroalkyl, hydroxyl, alkoxy, cyano, azido, halogen, nitro, SO₂, SO₃, thioalkyl or amino.

In naturally occurring vitamin B₁₂, there is an α-D-5,6-dimethylbenzimidazolyl ribose 3′-phosphate that is bound through the phosphate to the B₁₂ moiety and coordinated to the cobalt ion. In a modified TC- or IF-binding agent, the M-sugar component is likewise in an α-D configuration, although other configurations (i.e. α-L, β-D and β-L) are also possible.

One of the biologically active forms of vitamin B₁₂ has a 5′-deoxyadenosyl moiety in the X position. Vitamin B₁₂ catalysis occurs via the detachment and reattachment of the methylene radical at the 5′-deoxy position of the adenosyl moiety. In one embodiment, the selected substituent in the X position is capable of similar catalysis.

In one particular embodiment the linker used to attach the TC- or IF-binding agent and the active agent is a polyamine such as spermine or spermidine.

In another embodiment X comprises the residue of 5′-deoxyadenosine.

In one embodiment, the TC- or IF-binding agent comprises one or more active agents at each of one or more of the b-, d- or e- cobalamin positions, linked directly or through a linker and preferably through the b-position.

In another embodiment the TC- or IF-binding agent of the present invention comprises one or more active agents at M, V⁸ or G¹.

In yet another embodiment, X is NO. NO can be administered for wound healing or other known therapeutic functions of this moiety.

In still another embodiment, the active agent of the present invention comprises a radionuclide.

In still another embodiment, the active agent of the present invention does not comprise a radionuclide.

In one embodiment, the compound of formula I can be understood to exclude compounds (and therapeutic methods using such compounds) in which:

X is cyano, hydroxyl, methyl, adenosine or L-T,

M is the residue of 5,6-dimethylbenzimidazole,

E is O,

K is C(OH)H,

G¹ is hydrogen,

Y¹, Y², Y³, Y⁴, Y⁵, Y⁶ and Y⁷ are O,

L is a direct bond or a multivalent linker derived from a dicarboxylic acid (C(O)OH-alkylene-C(O)OH), a diamine (NH₂-alkylene-NH₂), an amino-carboxylic acid (C(O)OH-alkylene-NH₂), an amino acid, a peptide or a polymer of one or amino acids,

J¹, J² and J³ are all hydrogen,

all of R¹, R², R⁴, R⁵, R⁸, R⁹, R¹¹, R¹² and R¹⁵ are methyl and all of R³, R⁶, R⁷, R¹⁰, R¹³ and R¹⁴ are hydrogen, and/or

V¹Z¹, V³Z³, V⁶Z⁶ and V⁷Z⁷ are amino.

The invention also provides intermediates disclosed herein that are useful in the preparation of compounds of the present invention as well as synthetic methods for preparing the compounds of the invention.

The invention also provides a pharmaceutical composition comprising a compound of the invention, or its pharmaceutically acceptable salt or prodrug therein, and a pharmaceutically acceptable carrier or diluent.

The present invention also provides a method of preventing or treating an infection in a host, preferably, an animal, and even more preferably a human, comprising administering to the host a therapeutic amount of a TC- or IF-binding agent, or its pharmaceutically acceptable salt or prodrug therein, which comprises an antibiotic.

The present invention also provides a method of preventing, treating and/or imaging an infection in a host, preferably, an animal, and even more preferably a human, comprising administering to the animal an effective amount of a TC- or IF-binding agent, or its pharmaceutically acceptable salt or prodrug therein, which comprises an antibiotic and/or an imaging agent, and optionally detecting the presence of the compound.

The present invention also provides a method of imaging an infection in a host, preferably, an animal, and even more preferably a human, comprising administering to the animal a detectable amount of a TC- or IF-binding agent, or its pharmaceutically acceptable salt therein, which comprises an imaging agent and detecting the presence of the compound.

The invention also provides a method of preventing or treating an infectious disease (e.g. bacterial infections) in a host, preferably, an animal, and even more preferably a human, comprising administering to the host a therapeutic amount of a pharmaceutical composition comprising a TC- or IF-binding agent linked to an antibiotic, or its pharmaceutically acceptable salt or prodrug therein, and a pharmaceutically acceptable carrier.

The invention also provides a method of preventing, treating and/or imaging an infectious disease (e.g. bacterial infections) in a host, preferably, an animal, and even more preferably a human, comprising administering to the host an effective amount of a pharmaceutical composition comprising a TC- or IF-binding agent linked to an antibiotic and or an imaging agent, or its pharmaceutically acceptable salt or prodrug therein, and a pharmaceutically acceptable carrier, and optionally detecting the presence of the compound.

The invention also provides a method of imaging an infectious disease (e.g. bacterial infections) in a host, preferably, an animal, and even more preferably a human, comprising administering to the host a detectable amount of a pharmaceutical composition comprising a TC- or IF-binding agent linked to an imaging agent, or its pharmaceutically acceptable salt or prodrug therein, and a pharmaceutically acceptable carrier and detecting the presence of the compound.

The invention also provides a compound of the present invention for use in medical therapy.

The invention also provides the use of a TC- or IF-binding agent linked to an antibiotic, or its pharmaceutically acceptable salt or prodrug therein, for the treatment or prophylaxis of an infection in a host (e.g. an animal, preferably a human).

The invention also provides the use of a TC- or IF-binding agent linked to an antibiotic and/or an imaging agent, or its pharmaceutically acceptable salt or prodrug therein, for the treatment, prophylaxis or diagnosis of an infection in a host (e.g. an animal, preferably a human).

The invention also provides the use of a TC- or IF-binding agent linked to an imaging agent, or its pharmaceutically acceptable salt or prodrug therein, for the diagnosis of an infection in a host (e.g. an animal, preferably a human).

The invention also provides the use of a TC- or IF-binding agent linked to an antibiotic, or its pharmaceutically acceptable salt or prodrug therein, in the manufacture of a medicament for the treatment or prophylaxis of an infection in a host (e.g. an animal, preferably a human).

The invention also provides the use of a TC- or IF-binding agent linked to an antibiotic and/or an imaging agent, or its phannaceutically acceptable salt or prodrug therein, in the manufacture of a medicament for the treatment, prophylaxis or diagnosis of an infection in a host (e.g. an animal, preferably a human).

The invention also provides the use of a TC- or IF-binding agent linked to an imaging agent, or its pharmaceutically acceptable salt or prodrug therein, in the manufacture of a medicament for the diagnosis of an infection in a host (e.g. an animal, preferably a human).

### BRIEF DESCRIPTION OF THE FIGURES

FIG. 1 depicts the structure of cobalamin wherein X is CN (cyano), OH, CH₃ or adenosyl.

FIG. 2 illustrates a proposed synthesis of cyanocobalamin-leucine-antibiotic conjugates of the present invention.

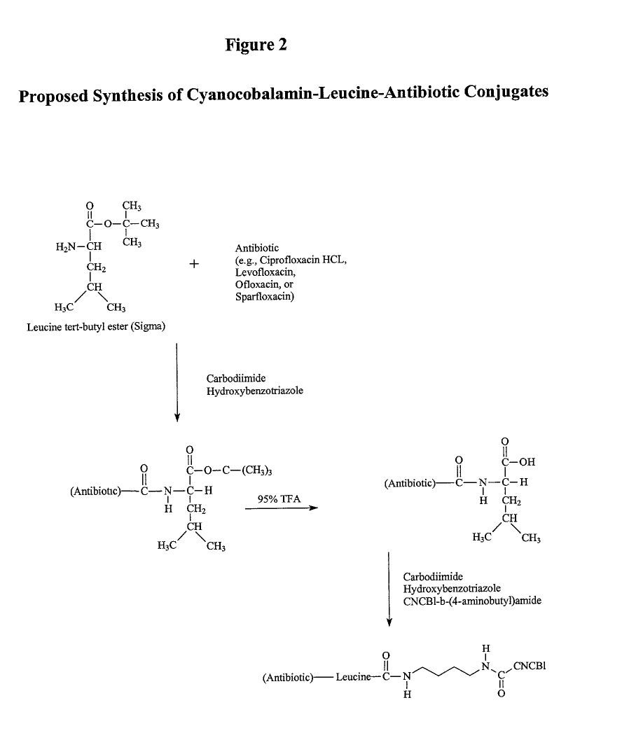

### DETAILED DESCRIPTION OF THE INVENTION

In one embodiment, a compound is provided that includes a transcobalamin- or intrinsic factor-binding agent (also referred to herein as a TC- or IF-binding agent) linked to an antibiotic, or an active residue thereof, or its pharmaceutically acceptable salt or prodrug thereof. In one example, the transcobalamin- or intrinsic factor-binding agent is a vitamin B₁₂ carrier that is covalently linked directly or via a spacer group to the antibiotic. In an alternative embodiment, the transcobalamin- or intrinsic factor-binding agent that is covalently linked to the antibiotic has the chemical structure indicated in formula I. The transcobalamin- or intrinsic factor -binding agent can be covalently linked to the antibiotic via conventional chemical processes. It has been discovered that such compounds will localize in or near the infectious disease, allowing efficient therapy.

In another embodiment, infectious diseases are diagnosed and or mapped by the use of a compound that includes a transcobalamin- or intrinsic factor -binding agent linked to a detectable radionuclide (e.g. metallic radioisotope or non-metallic radioisotope) or paramagnetic metal atom, or its pharmaceutically acceptable salt, which will localize in or near an infectious disease. It has been discovered that a compound wherein a TC- or IF-binding agent is linked to a residue of an imaging agent or its pharmaceutically acceptable salt will localize in or near an infectious disease.

In a preferred embodiment, the antibiotic and/or imaging agent and the TC- or IF-binding agent or its pharmaceutically acceptable salt or prodrug thereof, is delivered to the site of unwanted infection in a manner that bypasses or at least does not rely on, the gastrointestinal route of absorption via the vitamin B₁₂ intrinsic factor binding protein. Preferred modes of administration are parenteral, intraperitoneal, intravenous, intradermal, epidural, intraspinal, intrasternal, intra-articular, intra-synovial, intrathecal, intra-arterial, intracardiac, intramuscular, intranasal, subcutaneous, intraorbital, intracapsular, topical, transdermal patch, via rectal, vaginal or urethral administration including via suppository, percutaneous, nasal spray, surgical implant, internal surgical paint, infusion pump or via catheter. In one embodiment, the agent and carrier are administered in a slow release formulation such as a direct tissue injection or bolus, implant, microparticle, microsphere, nanoparticle or nanosphere.

In an alternative embodiment, it has been discovered that an agent for the treatment of infection can be highly and effectively absorbed into a site of unwanted infection by direct or indirect attachment to a compound that binds to the intrinsic factor (IF-binding agent), wherein the IF-binding agent and active agent are administered parenterally, for example, using any of the methods listed above.

In another embodiment, the TC- or IF-binding agent and the antibiotic or imaging agent is combined with either intrinsic factor or a transcobalamin carrier protein or both and administered parenterally, for example, via intravenous, intramuscular, direct injection or catheter, to the afflicted location.

The TC-or IF-binding agent and antibiotic or imaging agent useful to image sites of infectious diseases in the body, can optionally be joined by means of a di- or multi-valent linking moiety. The linker used to join the TC- or IF-binding agent and the active agent preferably has a single molecular weight and does not exhibit a molecular weight distribution, for example as found in most polymers. The linker can range in size from small to large molecular weight, as long as there is not a distribution of weights in the linker. It is important to strictly control the uniformity of size of the conjugate for predictability of therapeutic performance.

The linkers preferably have a molecular weight below about 2000, more preferably below about 1900 or 1800 and even more preferably below about 1500 or 1000.

Thus, in one embodiment the invention provides an antibiotic or an imaging conjugate having a high specificity for sites of infectious diseases, comprising (1) a TC- or IF-binding agent and (2) an antibiotic or an imaging agent linked directly or through a linker to the TC- or IF-binding agent, wherein the linker has either (i) a unimodal (i.e. single) and defined molecular weight or (ii) a molecular weight less than about 2000 and preferably, below 1900, 1800 or 1500.

In another embodiment, the invention provides a non-oral pharmaceutical formulation comprising an antibiotic or an imaging conjugate having a high specificity for sites of infectious diseases, comprising (1) a TC- or IF-binding agent and (2) an antibiotic or an imaging agent linked directly or through a linker to the TC- or IF-binding agent.

In one embodiment, the TC- or IF-binding agent is any moiety that will bind to a transcobalamin receptor and is able to be linked to an antibiotic or an imaging agent. Methods for the assessment of whether a moiety binds the TC receptor are known and include those described by Pathare et al. (1996) Bioconjugate Chem. 7, 217-232; and Pathare et al. Bioconjugate Chem. 8, 161-172. An assay that assesses binding to a mixture of transcobalamin I and II receptors is found in Chaiken et al. Anal. Biochem. 201, 197 (1992). An unsaturated vitamin B₁₂ binding capacity (UBBC) assay to assess the in vitro binding of the conjugate to the transcobalamin proteins is described by D. A. Collins and H. P. C. Hogenkamp in J. Nuclear Medicine, 1997, 38, 717-723. See also Fairbanks, V. F. Mayo Clinical Proc. 83, Vol 58, 203-204.

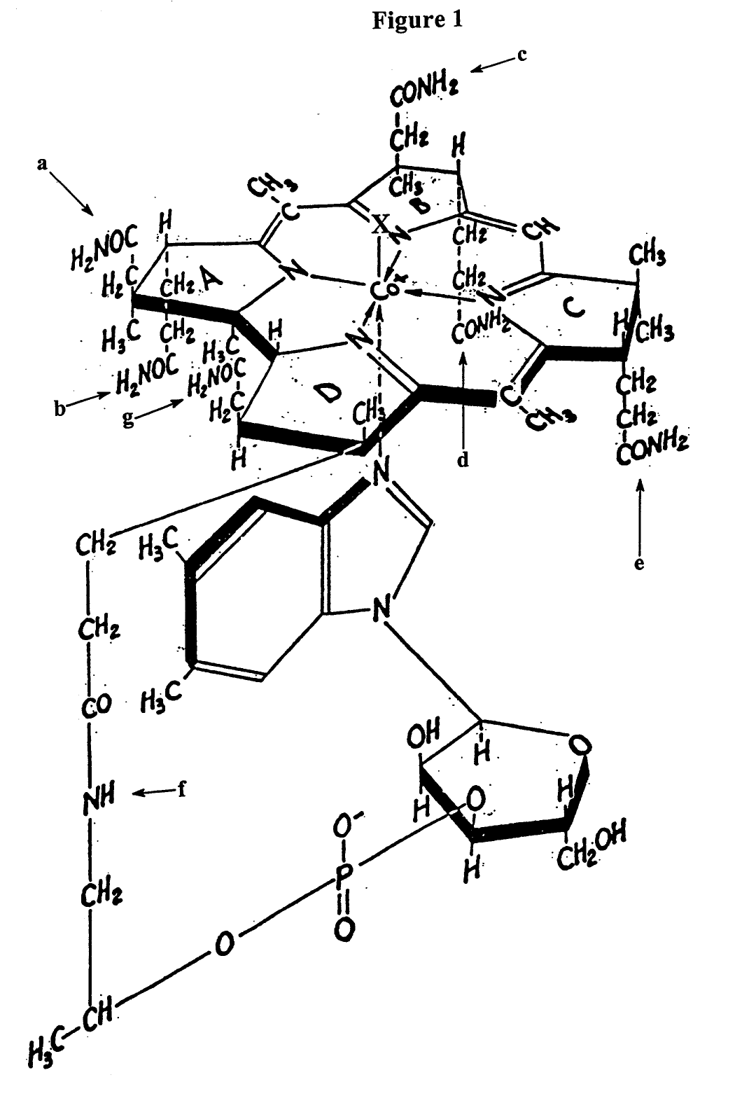

The imaging agent is preferably bound directly or indirectly through an amide residue at the b-position, as illustrated in FIG. 1.

In one embodiment, the agent and carrier are administered in a slow release formulation such as an implant, bolus, microparticle, microsphere, nanoparticle or nanosphere. Nonlimiting examples of sustained release compositions include semi-permeable polymer matrices in the form of shaped articles, e.g. films, microcapsules or microspheres. Sustained release matrices include, for example, polylactides (U.S. Pat. No. 3,773,919), copolymers of L-glutamic acid and γ-ethyl-L-glutarnate (Sidman et al., Biopolymers 22:547-556, 1983) or poly-D-(−)-3-hydroxybutyric acid (EP 133,988). Sustained release compositions also include one or more liposomally entrapped compounds of formula I. Such compositions are prepared by methods known per se, e.g. as taught by Epstein et al. Proc. Natl. Acad. Sci. USA 82:3688-3692, 1985. Ordinarily, the liposomes are of the small (200-800 Å) unilamellar type in which the lipid content is greater than about 30 mol % cholesterol, the selected proportion being adjusted for the optimal therapy.

A number of sustained-release implants are known in the art. Most implants are “matrix” type and comprise an active compound dispersed in a matrix of a carrier material. The carrier material may be either porous or non-porous, solid or semi-solid and permeable or impermeable to the active compound. Matrix devices are typically biodegradable, i.e. they slowly erode after administration. Alternatively, matrix devices may be nondegradable and rely on diffusion of the active compound through the walls or pores of the matrix. Matrix devices are preferred for the applications contemplated herein.

Thus, in one embodiment the invention provides a surgical implant for localized delivery of an active agent comprising the cobalamin conjugate of the present invention and a biodegradable binder. The implant preferably is capable of releasing and delivering the cobalamin conjugate to substantially all of an area of clear margin that results from a surgical resection and is also preferably capable of releasing the cobalamin conjugate at a substantially constant rate. In another embodiment the invention provides a method of delivering an imaging agent to an area of clear margin following a surgical resection comprising (i) providing an implant comprising a TC- or IF-binding agent linked to an imaging agent and a biodegradable binder; and (ii) placing the implant into a void created by surgical resection.

The surgical implant can exhibit a variety of forms. In one embodiment the implant is a bolus, comprising a viscous and deformable material capable of being shaped and sized before or during implantation to complement a void created by a surgical resection and sufficiently deformable upon implantation to contact substantially all of an area of clear margin. The surgical implant can also comprising a plurality of capsules that can be poured into the void created by a surgical resection. These capsules will contain the cobalamin conjugate and a suitable binder. Because they are flowable, they can be poured into the void created by a surgical lumpectomy and thereby contact substantially all of the areas of clear margin.

Many suitable compositions for the implant are known and can be used in practicing the invention. Such compositions are described in, for example, Chasin et al. Biodegradable Polymers as Drug Delivery Systems, Marcel Dekker Inc., NY, ISBN 0-8247-8344-1. Preferable compositions are pharmaceutically acceptable, biodegradable and meet the particular release profile characteristics that are required to achieve the administration regime involved.

The implant typically comprises a base composition that acts as a matrix to contain and hold the contents of the implant together. The base composition can, in turn, comprise one or more constituents. Examples of base compositions include polymers and copolymers of anhydrides or thoester, lactic acid, glycolic acid, dioxonane, trimethylene carbonate, ε-caprolactone, phosphazene and glyceryl monostearate.

In one embodiment the base composition for the matrix comprises a polyanhydride, which can be synthesized via the dehydration of diacid molecules by melt condensation. Degradation times can be adjusted from days to years according to the hydrophobicity of the monomer selected. The materials degrade primarily by surface erosion and possess excellent in vivo compatibility. In one embodiment the polyanhydride is formed from sebasic acid and hexadecandioic acid (poly(SA-HDA anhydride). Wafer-like implants using this base composition have been approved for use in brain cancer, as Giadel®, by Guilford Pharmaceuticals.

The implant optionally can comprise erosion and biodegradation enhancers that facilitate the erosion of the matrix, the dissolution of the core composition or the uptake of the core composition via metabolic processes. Particularly suitable erosion and biodegradation enhancers are biodegradable in biological fluids and biocompatible. Hydrophilic constituents are typical, because they are capable of enhancing the erosion of the implant in the presence of biological fluids. For example, K. Juni et al., Chem. Pharm. Bull., 33, 1609 (1985) disclose that the release rate of bleomycin from polylactic acid microspheres is greatly enhanced by incorporating fatty acid esters into the microspheres. Other exemplary hydrophilic constituents are described, for example, in Wade & Weller, Handbook of pharmaceutical Excipients (London: Pharmaceutical Press; Washington D.C.: American Pharmaceutical Ass'n 1995) and include the polyethylene glycols (“PEGs”), propylene glycol (“PG”), glycerin and sorbitol.

Surfactants further enhance the erosion of the matrix and the release of the drug. Surfactants are generally capable of increasing the wettability and the solubility of the base composition in biological fluids and thereby causing the disintegration and erosion of the implant. Surfactants can also help to break down the core composition matrix when, for example, the method of forming the dosage form has reduced the solubility of any of the constituents. Surfactants can also improve the uptake of the dosage forms into the bloodstream. Suitable surfactants include, for example, glyceryl based surfactants such as glyceryl monooleate and glyceryl monolaurate, poloxamers such as Pluronic F127 and polysorbates such as polyoxyethylene sorbitan monooleate (“Tween 80”).

The implant could also include components that retard the rate at which the implant erodes or biodegrades (erosion and/or biodegradation retardants). Hydrophobic constituents are a particularly suitable class of components for retarding the rate at which the outer layer biodegrades. Suitable hydrophobic constituents are described, for example, in the Handbook of Pharmaceutical Excipients, the disclosure from which being hereby incorporated by reference. Exemplary hydrophobic constituents include peanut oil, olive oil and castor oil.

Any proportions or types of constituents can be chosen that effectively achieve a desired release profile and thereby carry out the prescribed administration regime. The most desirable base compositions generally release the drug substantially continuously and biodegrade completely shortly after substantially all of the drug has been effectively released. The amount of drug included in the dosage forms is determined by the total amount of the drug to be administered and the rate at which the drug is to be delivered. The total amount of the drug to be delivered is determined according to clinical requirements and in keeping with the considerations that typically inform drug dosage determinations in other contexts. The surgical implant also can contain one or more other drugs having therapeutic efficacy in the intended applications, such as an antibiotic, an analgesic or an anesthetic.

In one embodiment, the TC- or IF-binding agent/active agent comprises at least one radionuclide.

In one embodiment, the TC- or IF-binding agent/active agent does not comprise a radionuclide.

In yet another embodiment, a TC- or IF-binding agent attached to a radiodiagnostic can be used in radionuclide detection of an infection, to detect and localize infections, especially sepsis, with or without localized signs or symptoms, or a fever of undetermined origin (FUO's). In this embodiment, the TC-binding or IF-binding agent and radiodiagnostic are administered, preferably via injection, to a site circumferental to the afflicted area in the body. The radiodiagnostic is preferentially taken up by infected cells due to the presence of the TC-binding or IF-binding agent and then is monitored in its normal course of travel in the body. This technique is especially useful in the detection of opportunistic lung infections found in immuno-compromised patients, such as an AIDS patient or patient who has undergone organ transplant. Scanning, therefore, is useful in staging and as a prognostic indicator that may obviate the need for bronchoscopy in some patients.

I. Definitions

Specific and preferred values listed below for radicals, substituents and ranges, are for illustration only; they do not exclude other defined values or other values within defined ranges for the radicals and substituents.

The following definitions are used, unless otherwise described: halo is fluoro, chloro, bromo or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho- fused bicyclic carbocyclic radical having about nine to ten ring atoms in which at least one ring is aromatic.

Specifically, (C₁-C₆)alkyl can be methyl, ethyl, propyl, isopropyl, butyl, iso-butyl, sec-butyl, pentyl, 3-pentyl, hexyl, heptyl, octyl, nonyl, decyl, undecyl, dodecyl, tridecyl or tetradecyl.

Specifically, (C₂-C₂₄)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1- hexenyl, 2-hexenyl, 3-hexenyl, 4-hexenyl, 5-hexenyl, heptenyl, octenyl, nonenyl, decenyl, undecenyl, dodecenyl, tridecenyl or tetradecenyl. Specifically, (C₂-C₂₄)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, heptynyl, octynyl, nonynyl, decynyl, undecynyl, dodecynyl, tridecynyl or tetradecynyl.

Specifically “aryl” can be phenyl, indenyl or naphthyl.

Specifically (C₃-C₈)cycloalkyl can be cyclopropyl, cyclobutyl, cyclcopentyl, cyclohexyl, cycloheptyl or cyclooctyl.

As used herein, an “amino acid” is a natural amino acid residue (e.g. Ala, Arg, Asn, Asp, Cys, Glu, Gln, Gly, His, Hyl, Hyp, Ile, Leu, Lys, Met, Phe, Pro, Ser, Thr, Trp, Tyr and Val) in D or L form, as well as unnatural amino acid (e.g. phosphoserine; phosphothreonine; phosphotyrosine; hydroxyproline; gamma-carboxyglutamate; hippuric acid; octahydroindole-2-carboxylic acid; statine; 1,2,3,4,-tetrahydroisoquinoline-3-carboxylic acid; penicillamine; ornithine; citruline; α-methyl-alanine; para-benzoylphenylalanine; phenylglycine; propargylglycine; sarcosine; and tert-butylglycine) residue having one or more open valences. The term also comprises natural and unnatural amino acids bearing amino protecting groups (e.g. acetyl, acyl, trifluoroacetyl or benzyloxycarbonyl), as well as natural and unnatural amino acids protected at carboxy with protecting groups (e.g. as a (C₁-C₆)alkyl, phenyl or benzyl ester or amide). Other suitable amino and carboxy protecting groups are known to those skilled in the art (See for example, T. W. Greene, Protecting Groups In Organic Synthesis; Wiley: New York, 1981; D. Voet, Biochemistry, Wiley: New York, 1990; L. Stryer, Biochemistry., (3rd Ed.), W. H. Freeman and Co.: New York, 1975; J. March, Advanced Organic Chemistry. Reactions, Mechanisms and Structure, (2nd Ed.), McGraw Hill: New York, 1977; F. Carey and R. Sundberg, Advanced Organic Chemistry, Part B: Reactions and Synthesis, (2nd Ed.), Plenum: New York, 1977; and references cited therein). According to the invention, the amino or carboxy protecting group can also comprise a non-metallic radionuclide (e.g. Fluorine-18, Iodine-123 or Iodine-124).

As used herein, a “peptide” is a sequence of 2 to 25 amino acids (e.g. as defined hereinabove) or peptidic residues having one or more open valences. The sequence may be linear or cyclic. For example, a cyclic peptide can be prepared or may result from the formation of disulfide bridges between two cysteine residues in a sequence. A peptide can be linked through the carboxy terminus, the amino terminus or through any other convenient point of attachment, such as, for example, through the sulfur of a cysteine. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right.

As used herein, “adenosyl” is an adenosine radical in which any synthetically feasible atom or groups of atoms have been removed, thereby providing an open valence. Synthetically feasible atoms that may be removed include the hydrogen atom of the hydroxy group at the 5′ position. Accordingly, adenosyl can conveniently be attached to the 6-position of a compound of formula I via the 5′position of adenosyl.

As used herein, the term “substantially free of” or “substantially in the absence of” refers to a composition that includes at least 85 or 90% by weight, preferably 95% to 98% by weight, and even more preferably 99% to 100% by weight, of the designated enantiomer of that TC- or IF-binding agent. In a preferred embodiment, in the methods and compounds of this invention, the compounds are substantially free their enantiomers.

Similarly, the term “isolated” refers to a composition that includes at least 85 or 90% by weight, preferably 95% to 98% by weight, and even more preferably 99% to 100% by weight, of the TC- or IF-binding agent, the remainder comprising other chemical species, including diastereomers or enantiomers.

The term “independently” is used herein to indicate that the variable that is independently applied varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

The term host, as used herein, refers to a unicellular or multicellular organism in which the infectious agent can replicate, including cell lines and animals, and preferably a human. Alternatively, the host can be carrying a part of the infectious agent's genome, whose replication or function can be altered by the compounds of the present invention. The term host specifically refers to infected cells, cells transfected with all or part of the infectious agent's genome and animals, in particular, primates (including chimpanzees) and humans. In most animal applications of the present invention, the host is a human patient. Veterinary applications, in certain indications, however, are clearly anticipated by the present invention (such as chimpanzees).

The term “pharmaceutically acceptable salt or prodrug” is used throughout the specification to describe any pharmaceutically acceptable form (such as an ester, mono-, di- or tri-phosphate ester, salt of an ester or a related group) of a TC- or IF-binding carrier, which, upon administration to a patient, provides the active compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess activity against infectious disease or are metabolized to a compound that exhibits such activity.

The term “residue” is used throughout the specification to describe any pharmaceutically acceptable form of an antibiotic agent, which, upon administration to a patient, does not inhibit the action of the antibiotic. As a non-limiting example, a pharmaceutically acceptable residue of an antibiotic is one that is modified to facilitate binding to the TC- or IF-binding agent, covalently, ionically or through a chelating agent, such that the modification does not inhibit the biological action of the antibiotic, in that it does not inhibit the drugs ability to modulate the infectious disease. In a preferred embodiment, the residue refers to the antibiotic with an open valence state such that covalent bonding to the compound is possible. This open valence state can be achieved by any means known in the art, including the methodology described herein. In a preferred embodiment, the open valence state is achieved through the removal of an atom, such as hydrogen, to activate a functional group.

II. Pharmaceutically Acceptable Salt or Prodrug Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including, sulfate, nitrate, bicarbonate and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the TC- or IF-binding agents described herein can be administered as a prodrug to increase the activity, bioavailability, stability or otherwise alter the properties of the carrier. A number of prodrug ligands are known. In general, alkylation, acylation or other lipophilic modification of the mono, di or triphosphate of the G¹ substituent on the five-membered “sugar-ring” moiety will increase the stability of the carrier. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed carriers to achieve a desired effect.

The G¹ substituent of the active carrier can also be provided as a 5′-phosphoether lipid or a 5′-ether lipid, as disclosed in the following references, which are incorporated by reference herein: Kucera, L. S., N. Iyer, E. Leake, A. Raben, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch. 1992. “Greatly enhanced inhibition of human immunodeficiency virus type 1 replication in CEM and HT4-6C cells by 3′-deoxythymidine diphosphate dimyristoylglycerol, a lipid prodrug of 3,-deoxythymidine.” Antimicrob. Agents Chemother. 36:2025.2029; Hosetler, K. Y., L. M. Stutuniller, H. B. Lenting, H. van den Bosch, and D. D. Richman, 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into the TC- or IF-binding agent, preferably at the G¹ position of the carrier or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (Jun. 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign patent applications that disclose lipophilic substituents that can be attached to the carrier of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

III. Nonlimiting Examples of Infectious Diseases

“Infectious diseases” or “infections” include, e.g. acute lower respiratory infections (e.g. pneumonia), lower urinary tract infections (UTI), tuberculosis (TB), Lyme's disease, malaria, meningitis, meningitis cause by Neisseria meningitis, hepatitis, measles, neonatal tetanus, diarrheal diseases (e.g. including cholera, typhoid and dysentery), whooping cough (pertussis), intestinal worm diseases and sexually transmitted diseases.

Some of the causative agents and diseases associated with them, include Rotavirus, a major cause of infantile diarrhea worldwide; Cryuptosporidium parvum, a parasite which causes acute and chronic diarrhea worldwide; Cryptosporidium parvum, a parasite which causes acute and chronic diarrhea; Legionella pneumophila, the bacterium which causes potentially fatal Legionnaires' disease; Ebola virus, which causes hemorrhagic fever—fatal in up to 80% of cases; Hantaan virus, which causes potentially fatal hemorrhagic fever with renal syndrome; Campylobacter jejuni, a bacterium which causes diarrhea; Human T-lymphotropic virus I (HTLV-1), which causes lymphoma-leukemia; Escherichia coli O157:H7 strain of bacteria, which causes bloody diarrhea; HTLV-2 virus, which causes hairy cell leukemia; Helicobacter pylori, the bacterium associated with peptic ulcer disease and stomach cancer; Human immunodeficiency virus (HIV), which causes AIDS; Hepatitis E virus, which causes epidemics of jaundice in hot climates; Human herpes virus 6, which causes fever and rash; Hepatitis C virus, which causes liver cancer as well as liver disease; Guanarito virus, which causes Venezuelan hemorrhagic fever; Vibrio cholerae 0139, which causes epidemic cholera; Sabia virus, which causes Brazilian hemorrhagic fever; and Human herpes virus 8, associated with Kaposi's sarcoma in AIDS patients.

The compounds of the invention can optionally be administered in conjunction with one or more known antimicrobial agents. Suitable antimicrobial agents are disclosed hereinabove as “antibiotics.”

In cases where compounds of the invention are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, α-ketoglutarate and α-glycerophosphate. Suitable inorganic salts may also be formed, including hydrochloride, sulfate, nitrate, bicarbonate and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) sales of carboxylic acids can also be made.

The compounds of the present invention can be formulated as pharmaceutical compositions and administered to a mammalian host, such as a human patient in a variety of forms adapted to the chosen route of administration, i.e. orally or parenterally (e.g. by intravenous, intramuscular, intraperitoneal). Preferably, the compounds are administered perenterally.

The active compound may also be administered intravenously or intraperitoneally by infusion or injection. Solutions of the active compound or its salts can be prepared in water, optionally mixed with a nontoxic surfactant. Dispersions can also be prepared in Glycerol, liquid polyethylene glycols, triacetin and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, propylene glycol, liquid polyethylene glycols and the like), vegetable oils, nontoxic glyceryl esters and suitable mixtures thereof. the proper fluidity can be maintained, for example, by the formation of liposomes, by the maintenance of the required particle size in the case of dispersions or by the use of surfactants. Various antibacterial and antifingal agents, for example, parabens, chlorobutanol, phenol, sorbic acid, thimersol and the like, can bring about the prevention of the action of microorganisms. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated about, as required followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For illustration, suitable doses of a compound of the invention for use in therapy, in conjunction with neutron capture, include doses in the range of from about 0.1 μg to about 100 μg, e.g. from about 0.5 μg to about 50 μg or from about 0.5 μg to 15 μg per treatment. Suitable doses for use in therapy include doses in the range of from about 0.1 mg to about 50 g, e.g. from about 0.5 mg to about 10 g or from about 0.5 g to 2 g per treatment.

The desired dose may conveniently be presented in a single dose or as divided doses administered at appropriate intervals, for example, as two, three, four or more sub-does per day. The sub-dose itself may be further divided, e.g. into a number of discrete loosely spaced admninistrations.

The compound are preferably dissolved or dispersed in a nontoxic liquid vehicle, such as physiological saline or a similar aqueous vehicle, to the desired concentration. A preselected therapeutic unit dose is then administered to the test animal or human patient, by oral administration or ingestion or by parenteral administration, as by intravenous or intraperitoneal infusion or injection, to attain the desired in vivo concentration. Doses useful for treating infectious diseases can be derived from those found to be effective to treat infectious diseases in humans in vitro or in animal models, such as those described hereinbelow or from dosage of other vitamin B₁₂molecules, previously employed in animal therapy.

IV. TC- or IF-Binding Carrier

In one embodiment, the TC- or IF-binding agent is any ligand that will bind effectively to a vitamin B₁₂ transport protein (i.e. transcobalamin I, II or III or intrinsic factor) and which when appropriately linked to an antibiotic and/or an imaging agent and bound to a transport protein, will fit into a transcobalamin receptor. Methods for the assessment of whether a moiety binds the TC receptor are known and include those described by Pathare et al., Bioconjugate Chem. 1996, 7, 217-232; and Pathare, et al., Bioconjugate Chem. 8, 161-172. An assay that assesses binding to a mixture of transcobalamin I and II receptors is found in Chaiken, et al, Anal. Biochem. 1992, 201, 197. An unsaturated Vitamin B₁₂ binding capacity (UBBC) assay to assess the in vitro binding of the conjugate to the transcobalamin proteins is described by D. A. Collins and H. P. C. Hogenkamp in J. Nuclear Medicine, 1997, 38, 717-723. See also Fairbanks, V. F. Mayo Clinical Proc. 83, Vol 58, 203-204. See also Fairbanks, V. F. Mayo Clinical Proc. 83, Vol 58, 203-204. The ligand preferably displays a binding affinity to transcobalamin of at least 50% of the binding affinity displayed by vitamin B₁₂, more preferably at least 75% and even more preferably at least 90%.

In one embodiment the cobalamin conjugate of the present invention is represented by formula I or an enantiomer, diasteriomer, salt or pro-drug thereof:

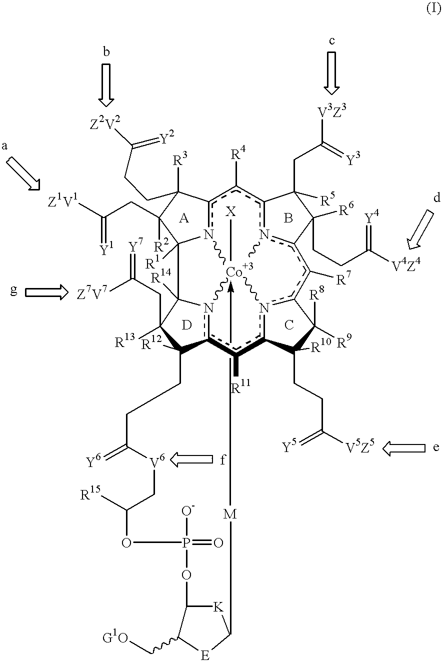

wherein:

the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;

the dotted line in the chemical structure indicates either a double or single bond such that the double bond does not over-extend the valence of the element (i.e. to give pentavalent carbons) and, in the case of a single bond, the valence is completed with hydrogen; and

wherein, in a preferred embodiment, the bonding and stereochemistry of the compound is the same as that of vitamin B₁₂ as it exists in nature.

X is hydrogen, cyano, halogen (Cl, F, Br or I), haloalkyl (including CF₃, CF₂CF₃, CH₂CF₃ and CF₂Cl), NO, NO₂, NO₃, phosphonate (including alkyl-P(O)₂OR¹⁵), PR¹⁵R¹⁶R¹⁷, NH₂, NR¹⁵R¹⁶, OH, OR¹⁵, SR¹⁵, SCN, N₃, OC(O)R¹⁵, C(O)₂R¹⁵, C(O)R¹⁵, OC(O)NR¹⁵R¹⁶, C(O)₂NR¹⁵R¹⁶, C(O)NR¹⁵R¹⁶, P(O)₂OR¹⁵, S(O)₂OR¹⁵, a purine or pyrimidine nucleoside or nucleoside analog, including adenosyl (preferably linked through a 5′-deoxy linkage) and 5-FU, alkyl, alkenyl, alkynyl, aryl, aralkyl, alkaryl, amino acid, peptide, protein, carbohydrate, heteroalkyl, heterocycle, heteroaryl or alkylheteroaryl. In one embodiment that is less preferred, X is L-T or L-T′.

M is a monovalent heterocycle or heteroaromatic, which is capable of binding to the adjacent sugar ring. M is preferably a benzimidazole, a 5- and/or 6- substituted benzimidazole, such as 5,6-dimethylbenzimidazole, 5-methyl-benzimidazole, 5-hydroxy-benzimidazole, 5-methoxy-benzimidazole, naphth-imidazole, 5-hydroxy-6-methyl-benz-imidazole or 5-methoxy-6-methyl-benz-imidazole; or a purine or pyrimidine including but not limited to adenine, 2-methyladenine, 2-methylmercaptoadenine, e-methylsulfinyladenine, 2-methyl-sulfonyladenine and guanine; or a phenol, such as phenol or p-cresol. The heterocycle or heteroaromatic can optionally be substituted with L-T or L-T′.

K is O, S, NJ¹, C(OH)H, CR¹⁰⁰R¹⁰¹ or C(R¹⁰⁰)V⁸Z⁸.

E is O, S, SO₂ or CH₂.

G¹ is hydrogen, alkyl, acyl, silyl, phosphate, L-T or L-T′.

Y¹, Y², Y³, Y⁴, Y⁵, Y⁶ and Y⁷ independently are O, S or NJ².

V¹, V², V³, V⁴, V⁵, V⁶, V⁷ and V⁸ independently are O, S, NJ³, CR¹⁰²R¹⁰³ or a direct bond.

Z¹, Z², Z³, Z⁴, Z⁵, Z⁷ and Z⁸ independently are R¹⁰⁴, L-T or L-T′.

Each L is independently a direct bond or a linker to one or more T or T′ moieties and that does not significantly impair the ability of the TC- or IF-binding agent to bind to a transcobalamin receptor.

Each T independently comprises an antibiotic agent, or a pharmaceutically acceptable residue thereof, optionally bound though a chelating moiety if necessary or desired. Each T′ independently comprises an imaging agent, optionally bound though a chelating moiety if necessary or desired. In one embodiment, T is an antibiotic for the treatment or prevention of infectious disease. In an alternate embodiment, T′ is an imaging agent for the diagnosis of infectious disease.

At least one of Z¹, Z², Z³, Z⁴, Z⁵, Z⁷, Z⁸, X, M and G¹ is independently L-T or L-T′. In a preferred embodiment, at least one of Z¹, Z², Z³, Z⁴, Z⁵, Z⁷, Z⁵ and G¹ is independently L-T, wherein T is independently an antibiotic. In another embodiment, the compound of formula I contain at least one T that is independently an antibiotic and at least one T′ that is independently an imaging agent. In a preferred embodiment, Z² comprises the sole L-T in the TC- or IF-binding agent.

J¹, J² and J³ independently are hydrogen, alkyl, alkenyl, alkynyl, alkaryl, cycloalkyl, aryl, cycloaryl, heteroalkyl, heterocycle, heteroaryl, hydroxyl, alkoxy or amine.

R¹, R², R³, R⁴, R⁵, R⁶, R⁷, R⁸, R⁹, R¹⁰, R¹¹, R¹², R¹³ and R¹⁴ independently are hydrogen, lower alkyl, lower alkenyl, lower alkynyl, lower cycloalkyl, heteroalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine.

R¹³ and R¹⁴ optionally can form a double bond.

R¹⁵, R¹⁶ and R¹⁷ are independently hydrogen, alkyl, alkenyl, alkynyl, aryl, alkaryl or aralkyl group, heteroalkyl, heterocycle or heteroaromatic.

R¹⁰⁰, R¹⁰¹, R¹⁰², R¹⁰³ and R¹⁰⁴ are independently hydrogen, alkyl, alkenyl, alkynyl, aryl, acyl, heteroaromatic, heteroaryl, heteroalkyl, hydroxyl, alkoxy, cyano, azido, halogen, nitro, SO₂, SO₃, thioalkyl or amino.

In naturally occurring vitamin B₁₂, there is an α-D-5,6-dimethylbenzimidazolyl ribose 3′-phosphate that is bound through the phosphate to the B₁₂ moiety and coordinated to the cobalt ion. In a modified vitamin B₁₂ TC- or IF-binding agent, the M-sugar component is likewise in an α-D configuration, although other configurations (i.e. α-L, β-D and β-L) are possible.

One of the biologically active forms of vitamin B₁₂ has a 5′-deoxyadenosyl moiety in the X position. Vitamin B₁₂ catalysis occurs via the detachment and reattachment of the methylene radical at the 5′-deoxy position of the vitamin.

In one particular embodiment the linker used to conjugate the TC- or IF-binding agent and the imaging agent is a polyamine such as spermine or spermidine.

Because vitamin B₁₂ is preferentially taken up in or near the infectious site, the TC-or IF-binding agent/active agent of the present invention provides a delivery system capable of targeting infections and selectively imaging a greater proportion of such infection in relation to healthy cells. A wide range of analogs and derivatives are capable of attaining these properties, as reflected by the above referenced chemical structure and variables.

The TC- or IF-binding agent can be modified in any manner that does not interfere with its fundamental ability to bind a transcobalamin transport protein and thereafter bind the TC receptor. In one embodiment, however, each variable on the vitamin B₁₂ structure independently either (i) retains its natural vitamin B₁₂ structure, (ii) imparts an imaging agent and/or antibiotic to the cobalamin conjugate, (iii) renders the cobalamin conjugate more water soluble or more stable, (iv) increases the bioavailability of the carrier; (v) increases or at least does not decrease the binding affinity of the carrier for the TC-binding or IF-binding protein over vitamin B_{12;} or (vi) imparts another characteristic that is desired for pharmaceutical or diagnostic performance.

The imaging agent can be linked to the TC-binding or IF-binding moiety through a number of positions, including any of the V-Z moieties, the X moiety, the M moiety, the K moiety and/or the G¹ moiety, though as mentioned above at least one of Z¹, Z², Z³, Z⁴, Z⁵, Z⁷, Z⁸, M and G¹ moieties comprises an imaging agent. In one embodiment an imaging agent is linked to the TC- or IF-binding agent through Z², Z⁴, and/or Z⁵ (i.e. one or more of Z², Z⁴ and Z⁵ is L-T and T is an imaging agent). In a more particular embodiment an imaging agent is linked to the TC- or IF-binding agent through the Z² moiety (i.e. Z² is L-T and T is an imaging agent). In each of the foregoing embodiments, the Z moiety or moieties not containing an imaging agent preferably retain its natural vitamin B₁₂ configuration, in which VZ is NH₂. Alternatively, the Z moieties not containing an imaging agent may comprise a secondary or tertiary amino analog of NH₂ substituted by one or two of J¹.

In any Z¹, Z², Z³, Z⁴, Z⁵, Z⁶, Z⁷, Z⁸, X, M or G¹ moieties through which an imaging agent is linked, it will be understood that such moiety may comprise more than one imaging agent or a combination of imaging agents, i.e. each T can independently comprise the residue of one or more imaging agents bound to L through one or more chelating moieties. More specifically, in a series of embodiments, each T can comprise 1, 2, 3, 4, 5, 6, 7, 8, 9 or 10 imaging agents bound through one or more chelating moieties.

R¹, R², R³, R⁴, R⁵, R⁶, R⁷, R⁸, R⁹, R¹⁰, R¹¹, R¹² and R¹³ independently represent moieties that do not interfere with binding between the compound and the transcobalamin transport protein or receptor. Vitamin B1₂ can be modified through these moieties to modulate physical properties of the molecule, such as water solubility, stability or λₘₐₓ, Preferred groups for enhancing water solubility include heteroalkyl, amino, C₁₋₆ alkylamino, C₁₋₆ alcohol, C₁₋₆ carboxylic acid and SO₃⁻.

In another embodiment, one, some or all of R¹, R², R³, R⁴, R⁵, R⁶, R⁷, R⁸, R⁹, R¹⁰, R¹¹, R¹² and R¹³ independently assume their natural roles in vitamin B_{12.} Thus, one, some or all of R¹, R², R⁴, R⁵, R⁸, R⁹, R¹¹, R¹² and R¹⁵ are independently methyl in one embodiment and one, some or all of R³, R⁶, R⁷, R¹⁰, R¹³ and R¹⁴ are independently hydrogen.

In another embodiment, one, some or all of Y¹, Y², Y³, Y⁴, Y⁵, Y⁶ and Y⁷ assume their natural roles in vitamin B₁₂ and are O. Similarly, in another embodiment V⁶ assumes its natural role in vitamin B₁₂ and is NH or a primary amine analog thereof substituted by J¹.

In still another embodiment, position X assumes its natural role in vitamin B₁₂, i.e. as cyano, hydroxyl, methyl or 5′-deoxyadenosyl, most preferably 5′-deoxyadenosyl.

In another embodiment M is the radical of a purine or pyrimidine base. In another embodiment M is the radical of adenosine, guanine, cytosine, uridine or thymine. In still another embodiment M is the radical of 5,6-dimethylbenzimidazole.

In still another embodiment K is CH(OH).

In yet another embodiment E is O.

In another embodiment G¹ is OH.

In still another embodiment, all constituents of the conjugate assume their natural roles in vitamin B₁₂, except for the moieties through which any imaging agents are linked. The imaging agent(s) are preferably linked to the vitamin B₁₂ structure through Z², Z⁴ and/or Z⁵ and even more preferably through the Z² moieties.

V. Linkers

As noted above, L is the residue of a linker molecule that conjugates one or more imaging agents to the TC ligand. The structure of the linker from which L is derived (in any one of the Z¹, Z², Z³, Z⁴, Z⁵, Z⁶, Z⁷, X, M or G¹ moieties) is not crucial, provided it does not significantly impair the ability of the conjugate to bind to the transcobalamin or IF transport protein or receptor. L is preferably any multivalent molecule (divalent or greater) that does not significantly impair the ability of the TC carrier to bind to the transcobalamin transport protein or receptor. The ability of vitamin B₁₂ or any other TC-binding carrier to bind to the transcobalamin transport protein or receptor is “significantly impaired” when attaching a linking moiety to the B₁₂ or TC-binding carrier lessens the affinity of the vitamin B₁₂ or the TC-binding carrier for the transcobalamin transport protein to which the vitamin B₁₂ or TC-binding carrier is most readily bound by 50% or more. The unsaturated vitamin B₁₂ binding capacity (UBBC) assay described by D. A. Collins and H. P. C. Hogenkamp in J. Nuclear Medicine, 1997, 38, 717-723 can be used to compare the relative affinities of ligands for this receptor.

In one embodiment the linker is of precise molecular weight and does not posses a molecular weight distribution. In one embodiment, the linker has a molecular weight less than about 2,500, 2,000, 1900, 1800, 1,500, 1,000 or 500.

A particularly preferred linker is one having multiple sites for conjugation to one or more imaging agents, wherein the linker has a unimodal molecular weight. Recombinant protein production techniques can be employed to obtain poly(amino acid) linkers of substantially constant molecular weight.

In one embodiment the linker is an amino acid or a polymer or peptide formed from a plurality of amino acids. The polymer or peptide can be derived from one or more amino acids. The amino acid, poly(amino acid) or peptide can link T to V through the carboxy terminus or the amino terminus. The amino acid residue, peptide residue or poly(amino acid) residue can conveniently be linked to V and T through an amide (e.g. —N(R)C(—O)— or —C(═O)N(R)—), ester (e.g. —OC(═O)— or —C(═O)O—), ether (e.g. —O—), ketone (e.g. —C(═O)—), thioether (e.g. —S—), sulfinyl (e.g. —S(O)—), sulfonyl (e.g. —S(O)₂—) or a direct (e.g. C—C bond) linkage, wherein each R is independently H or (C₁-C₁₄) alkyl.

Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right, but are meant to also include the opposite flow. Particularly suitable peptides and poly(amino acids) comprise from 2 to about 20 amino acids, from 2 to about 15 amino acids or from 2 to about 12 amino acids.

One exemplary poly(amino acid) is poly-L-lysine ((—NHCH((CH₂)₄—NH₂)CO—)ₘ—Q, wherein Q is H, (C₁-C₁₄)alkyl or a suitable carboxy protecting group and m is from 2 to about 20, from about 5 to about 15 or from about 8 to about 11. The polylysine offers multiple primary amine sites to which active agents can be readily attached. Alternatively, the linkers can be formed with multiple cysteines, to provide free thiols or multiple glutamates or aspartates, to provide free carboxyls for conjugation using suitable carbodiimides. Similarly the linker can contain multiple histidines or tyrosines for conjugation. Other exemplary poly(amino acid) linkers are poly-L-glutamic acid, poly-L-aspartic acid, poly-L-histidine, poly-L-ornithine, poly-L-serine, poly-L-threonine, poly-L-tyrosine, poly-L-lysine-L-phenylalanine or poly-L-lysine-L-tyrosine. When the linker is derived from a poly(amino acid) other than polylysine, the linker is, in a series of embodiments, prepared from 2 to about 30 amino acids, 5 to about 20 amino acids or 8 to about 15 amino acids.

In another particular embodiment L is a polyamine residue (having at least three amino moieties) of the following chemical structure: NR′(alkylene-NR′)ₙalkyleneNR′, wherein n is from 1 to 20, the carbon length of alkylene can vary within the n units and each R′ is independently hydrogen, lower alkyl or T. N is preferably from 1 to 10. Moreover, L preferably has a backbone along its longest length of no more than 100, 75, 50, 40, 30, 20 or 15 atoms. Exemplary polyamines from which L can be derived include spermine (H₂N(CH₂)₃NH(CH₂)₄NH(CH₂)₃NH₂), spermidine (H₂N(CH₂)₃NH(CH₂)₄NH₂), decamethylene tetraamine and pentamethylene hexamine. These linkers are a definite size and thus provide consistent and predictable targeting by the cobalamin conjugate, in addition to multiple binding sites for the imaging agent.

In another embodiment L is a diamine represented by the formula NH₂ (CH₂)ₓ NH₂, in which x is 2-20 and preferably 2-12. Thus, the linker can be prepared from 1,6-diaminohexane, 1,5-diaminopentane, 1,4-diaminobutane and 1,3-diaminopropane.

Other suitable linkers are formed from the covalent linkage of various water soluble molecules with amino acids, peptides, poly(amino acids), polyamines, polyoxyalkylenes, polyanhydrides, polyesters, polyamides, polyglycolides and diamines. Suitable water soluble molecules include, for example, polyethylene glycol and dicarboxylic monosaccharides such as glucaric acid, galactaric acid and xylaric acid.

Other suitable linkers include those represented by the formula HO(O)C(CH₂)ₓC(O)OH, in which x is 2-20 and preferably 2-12. Thus, the linker can be prepared from succinic acid, glutaric acid, adipic acid, suberic acid, sebacic acid, azelaic acid or maleic acid. Still other suitable linkers comprise carboxylic acid derivatives that yield an amide upon reaction with an amine. Such reactive groups include, by way of example, carboxylic acid halides such as acid chlorides and bromides; carboxylic acid anhydrides such as acetic anhydrides and trifluoroacetic anhydrides; esters such as p-nitrophenyl esters and N-hydroxysuccinimide esters; and imidazolides. Techniques for using such linkers are described in detail in Bodanszky, Principles of Peptide Synthesis, Springer Verlag, Berlin, 1984.

In one embodiment, the linker is modified to facilitate its conjugation either to V or T. Suitable molecules for modifying the linker include: disuccinimidyl suberate (DSS), bis(sulfosuccinimidyl) suberate (BSS), ethylene glycolbis(succinimidylsuccinate) (EGS), ethylene glycolbis(sulfosuccinimidyl-succinate) (Sulfo-EGS), p-aminophenylacetic acid, dithio-bis-(succinimidyl-propionate) (DSP), 3,3′-dithiobis-(sulfosuccinimidylpropionate) (DTSSP), disuccinimidyl tartarate (DST), disulfosuccinimidyl tartarate (Sulfo-DST), bis(2-(succinimidooxycarbonyloxy)-ethylene)sulfone (BSOCOES), bis(2-(sulfosuccinimidooxy-carbonyloxy)ethylene)sulfone (Sulfo-BSOCOES), dimethyl adipimidate.2HCl (DMA), dimethyl pimelimidate.2HCl (DMP) and dimethyl suberimidate.2HCl (DMS).

Biodegradable linkers

Various degradable linkers can be used to link the TC-binding or IF-binding moiety to the active agent. The desired linkers can degrade under biological conditions such as by enzymatic cleavage or by systemic pH or temperature. Alternatively, these linkers can be induced to degrade by external manipulation such as changes in pH, temperature, ultrasound, magnetic field, radiation (i.e. UV radiation) or light.

U.S. Pat. No. 5,639,885 entitled “Redox amino acids and peptides containing them;” U.S. Pat. No. 5,637,601 entitled “Anticholinergic compounds, compositions and methods of treatment;” U.S. Pat. No. 5,624,894 entitled “Brain-enhanced delivery of neuroactive peptides by sequential metabolism;” U.S. Pat. No. 5,618,826 entitled “Anticholinergic compounds, compositions and methods of treatment;” U.S. Pat. No. 5,618,803 entitled “Targeted drug delivery via phosphonate derivatives;” U.S. Pat. No. 5,610,188 entitled “Anticholinergic compounds, compositions and methods of treatment;” U.S. Pat. No. 5,525,727 entitled “Brain-specific drug delivery;” U.S. Pat. No. 5,418,244 entitled “Anticholinergic compounds, compositions and methods of treatment;” U.S. Pat. No. 5,413,996 entitled “Targeted drug delivery via phosphonate derivatives;” U.S. Pat. No. 5,389,623 entitled “Redox carriers for brain-specific drug delivery;” U.S. Pat. No. 5,296,483 entitled “Brain-specific analogues of centrally acting amines;” U.S. Pat. No. 5,258,388 entitled “Anticholinergic compounds, compositions and methods of treatment;” U.S. Pat. No. 5,231,089 entitled “Method of improving oral bioavailability of carbamazepine;” U.S. Pat. No. 5,223,528 entitled “Anticholinergic compounds, compositions and methods of treatment;” U.S. Pat. No. 5,187,158 Brain-specific drug delivery;” U.S. Pat. No. 5,177,064 entitled “Targeted drug delivery via phosphonate derivatives;” U.S. Pat. No. 5,155,227 entitled “Compounds for site-enhanced delivery of radionuclides;” U.S. Pat. No. 5,136,038 entitled “Radiopharmaceuticals and chelating agents useful in their preparation;” U.S. Pat. No. 5,087,618 entitled “Redox carriers for brain-specific drug delivery;” U.S. Pat. No. 5,079,366 entitled “Quaternary pyridinium salts;” U.S. Pat. No. 5,053,215 entitled “NMR-assayable ligand-labeled trifluorothymidine containing composition and method for diagnosis of HSV infection;” U.S. Pat. No. 5,024,998 entitled “Pharmaceutical formulations for parenteral use;” U.S. Pat. No. 5,017,618 entitled “Labile derivatives of ketone analogs of 3-substituted-1-alkylamino-2-propanols and their use as beta-adrenergic blockers;” U.S. Pat. No. 5,017,566 entitled “Redox systems for brain-targeted drug delivery;” U.S. Pat. No. 5,008,257 entitled “Brain-specific drug delivery;” U.S. Pat. No. 5,002,935 entitled “Improvements in redox systems for brain-targeted drug delivery;” U.S. Pat. No. 4,983,586 entitled “Pharmaceutical formulations for parenteral use;” U.S. Pat. No. 4,963,688 entitled “Compounds for site-enhanced delivery of radionuclides and uses thereof;” U.S. Pat. No. 4,963,682 entitled “Novel radiopharmaceuticals and chelating agents useful in their preparation;” U.S. Pat. No. 4,933,438 entitled “Brain-specific analogues of centrally acting amines;” U.S. Pat. No. 4,900,837 entitled “Brain-specific drug delivery of steroid sex hormones cleaved from pyridinium carboxylates and dihydropyridine carboxylate precursors;” U.S. Pat. No. 4,892,737 entitled “Composition and method for enhancing permeability of topical drugs;” U.S. Pat. No. 4,888,427 entitled “Amino acids containing dihydropyridine ring systems for site-specific delivery of peptides to the brain;” U.S. Pat. No. 4,880,921 entitled “Brain-specific drug delivery;” 35. U.S. Pat. No. 4,863,911 entitled “Method for treating male sexual dysfunction;” U.S. Pat. No. 4,829,070 entitled “Novel redox carriers for brain-specific drug delivery;” U.S. Pat. No. 4,824,850 entitled “Brain-specific drug delivery;” U.S. Pat. No. 4,801,597 entitled “Certain inositol-nicotinate ester derivatives and polyionic complexes therefore useful for treating diabetes meuitus, hyperlipidemia and lactic acidosis;” U.S. Pat. No. 4,771,059 entitled “Brain-specific analogues of centrally acting amines;” U.S. Pat. No. 4,727,079 entitled “Brain-specific dopaminergic activity involving dihydropyridine carboxamides, dihydroquinoline and isoquinoline carboxamides;” U.S. Pat. No. 4,540,564 entitled “Brain-specific drug delivery;” and U.S. Pat. No. 4,479,932 entitled “Brain-specific drug delivery” to Nicholas S. Bodor, et al., disclose several biodegradable linkers that target the brain. For example, a lipoidal form of dihydropyridine pyridinium salt redox carrier, DHC, linked to a centrally acting drug which can be reduced and biooxidized to pass through the blood brain barrier. The dihydropyridine nucleus readily and easily penetrates the blood brain barrier in increased concentrations; furthermore, the in vivo oxidation of the dihydropyridine moiety to the ionic pyridinium salts thereby prevents its elimination from the brain, while elimination from the general circulation is accelerated, resulting in a prolongedly sustained brain-specific drug activity. This dihydropyridine can be incorporated into the linkers set forth above for biodegradation.

Additionally U.S. Pat. No. 4,622,218 entitled “Testicular-specific drug delivery,” discloses linkers that can specifically deliver drugs to the testes in much the same manner and which can be used in the linkers of the present invention. The lipoidal form [D--DHC] of a dihydropyridine pyridinium salt redox carrier, e.g. 1,4-dihydrotrigonelline, penetrates the blood-testis barrier. Oxidation of the dihydropyridine carrier moiety in vivo to the ionic pyridinium salt type drug/carrier entity [D--QC]⁺ prevents elimination thereof from the testes, while elimination from the general circulation is accelerated, resulting in significant and prolongedly sustained testicular-specific drug activity.

Margerum, et al. in U.S. Pat. No. 5,976,493 discloses the use of polychelant compounds which are degradable in vivo to release excretable fragments for diagnostic imaging which also are suitable in the linkers of the present invention. These compounds contain a linker moiety which is metabolically cleavable to release macrocyclic monochelant fragments, wherein the macrocyclic skeleton preferably has 9 to 25 ring members and a biotolerable polymer, preferably a substantially monodisperse polymer. Other suitable linkers are disclosed, for example, in Krejcarek et al. (Biochemical and Biophysical Research Communications 77: 581 (1977)) (mixed anhydrides), Hnatowich et al. (Science 220: 613 (1983))(cyclic anhydrides), U.S. Pat. No. 5,637,684 to Cook, et al. (Phosphoramidate and phosphorothioamidate oligomeric compounds).

Other suitable biodegradable polymers from which the linker can be formed are the polyanhydrides and polyorthoesters, which take advantage of labile backbone linkages (see: Domb et al. Macromolecules, 22, 3200, 1989; and Heller et al. Biodegradable Polymers as Drug Delivery Systems, Dekker, NY: 1990). Other linker materials include hydrogels, such as the PEG-oligoglycolyl-acrylates disclosed in U.S. Pat. No. 5,626,863 to Hubbell et al. Other biodegradable linkers are formed from oligoglycolic acid is a poly(a-hydroxy acid), polylactic acid, polyeaprolactone, polyorthoesters, polyanhydrides and polypeptides.

Nonlimiting examples of U.S. Patents that describe controlled release formulations suitable for use as linking agents are: U.S. Pat. No. 5,356,630 to Laurencin et al. (Delivery System for Controlled Release of Bioactive Factors); ; U.S. Pat. No. 5,797,898 to Santini, Jr. et al. (Microchip Drug Delivery Devices); U.S. Pat. No. 5,874,064 to Edwards et al. (Aerodynamically Light Particles for Pulmonary Drug Delivery); U.S. Pat. No. 5,548,035 to Kim et al. (Biodegradable Copolymer as Drug Delivery Matrix Comprising Polyethyleneoxide and Aliphatic Polyester Blocks); U.S. Pat. No. 5,532,287 to Savage et al. (Radiation Cured Drug Release Controlling Membrane); U.S. Pat. No. 5,284,831 to Kahl et al. (Drug Delivery Porphyrin Composition and Methods); U.S. Pat. No. 5,741,329 to Agrawal et al. (Methods of Controlling the pH in the Vicinity of Biodegradable Implants); U.S. Pat. No. 5,820,883 to Tice et al. (Methods for Delivering Bioactive Agents into and Through the Mucosally-Associated Lymphoid Tissues and Controlling Their Release); U.S. Pat. No. 5,955,068 to Gouin et al. (Biodegradable polyanhydrides Derived from Dimers of Bile Acids and Use Thereof as Controlled Drug Release Systems); U.S. Pat. No. 6,001,395 to Coombes et al. (Polymeric Lamellar Substrate Particles for Drug Delivery); U.S. Pat. No. 6,013,853 to Athanasiou et al. (Continuous Release Polymeric Inplant Carriers); U.S. Pat. No. 6,060,582 to Hubbell et al. (Photopolymerizable Biodegradable Hydrogels as Tissue Contacting Materials and Controlled Release Carriers); U.S. Pat. No. 6,113,943 to Okada et al. (Sustained-Release Preparation Capable of Releasing a Physiologically Active Substance); and PCT Publication No. WO 99/59548 to Oh et al. (Controlled Drug Delivery System Using the Conjugation of Drug to Biodegradable Polyester); U.S. Pat. No. 6,123,861 (Fabrication of Microchip Drug Delivery Devices); U.S. Pat. No. 6,060,082 (Polymerized Liposomes Targeted to M cells and Useful for Oral or Mucosal Drug Delivery); U.S. Pat. No. 6,041,253 (Effect of Electric Field and Ultrasound for Transdermal Drug Delivery); U.S. Pat. No. 6,018,678 (Transdermal protein delivery or measurement using low-frequency sonophoresis); U.S. Pat. No. 6,007,845 Nanoparticles And Microparticles Of Non-Linear Hydrophilic-Hydrophobic Multiblock Copolymers; U.S. Pat. No. 6,004,534 Targeted Polymerized Liposomes For Improved Drug Delivery; U.S. Pat. No. 6,002,961 Transdermal Protein Delivery Using Low-Frequency Sonophoresis; U.S. Pat. No. 5,985,309 Preparation Of Particles For Inhalation; U.S. Pat. No. 5,947,921 Chemical And Physical Enhancers And Ultrasound For Transdermal Drug Delivery; U.S. Pat. No. 5,912,017 Multiwall Polymeric Microspheres; U.S. Pat. No.5,911,223 Introduction Of Modifying Agents Into Skin By Electroporation; U.S. Pat. No. 5,874,064 Aerodynamically Light Particles For Pulmonary Drug Delivery; U.S. Pat. No. 5,855,913 Particles Incorporating Surfactants For Pulmonary Drug Delivery; U.S. Pat. No. 5,846,565 Controlled Local Delivery Of Chemotherapeutic Agents For Treating Solid Tumors; U.S. Pat. No. 5,837,752 Semi-Interpenetrating Polymer Networks; U.S. Pat. No. 5,814,599 Transdermal Delivery Of Encapsulated Drugs; U.S. Pat. No. 5,804,178 Implantation Of Cell-Matrix Structure Adjacent Mesentery, Omentum Or Peritoneum Tissue; U.S. Pat. No. 5,797,898 Microchip Drug Delivery Devices; U.S. Pat. No. 5,770,417 Three-Dimensional Fibrous Scaffold Containing Attached Cells For Producing Vascularized Tissue In vivo; U.S. Pat. No. 5,770,193 Preparation Of Three-Dimensional Fibrous Scaffold For Attaching Cells To Produce Vascularized Tissue In vivo; U.S. Pat. No. 5,762,904 Oral Delivery Of Vaccines Using Polymerized Liposomes; U.S. Pat. No. 5,759,830 Three-Dimensional Fibrous Scaffold Containing Attached Cells For Producing Vascularized Tissue In vivo; U.S. Pat. No. 5,749,847 Delivery Of Nucleotides Into Organisms By Electroporation; U.S. Pat. No. 5,736,372 Biodegradable Synthetic Polymeric Fibrous Matrix Containing Chondrocyte For In vivo Production Of A Cartilaginous Structure; U.S. Pat. No. 5,718,921 Microspheres Comprising Polymer And Drug Dispersed There Within; U.S. Pat. No. 5,696,175 Preparation Of Bonded Fiber Structures For Cell Implantation; U.S. Pat. No. 5,667,491 Method For Rapid Temporal Control Of Molecular Transport Across Tissue; U.S. Pat. No. 5,654,381 Functionalized Polyester Graft Copolymers; U.S. Pat. No. 5,651,986 Controlled Local Delivery Of Chemotherapeutic Agents For Treating Solid Tumors; U.S. Pat. No. 5,629,009 Delivery System For Controlled Release Of Bioactive Factors; U.S. Pat. No. 5,626,862 Controlled Local Delivery Of Chemotherapeutic Agents For Treating Solid Tumors; U.S. Pat. No. 5,593,974 Localized Oligonucleotide Therapy; U.S. Pat. No. 5,578,325 Nanoparticles And Microparticles Of Non-Linear Hydrophilic-Hydrophobic Multiblock Copolymers; U.S. Pat. No. 5,562,099 Polymeric Microparticles Containing Agents For Imaging; U.S. Pat. No. 5,545,409 Delivery System For Controlled Release Of Bioactive Factors; U.S. Pat. No. 5,543,158 Biodegradable Injectable Nanoparticles; U.S. Pat. No. 5,514,378 Biocompatible Polymer Membranes And Methods Of Preparation Of Three Dimensional Membrane Structures; U.S. Pat. No. 5,512,600 Preparation Of Bonded Fiber Structures For Cell Implantation; U.S. Pat. No. 5,500,161 Method For Making Hydrophobic Polymeric Microparticles; U.S. Pat. No. 5,487,390 Gas-filled polymeric microbubbles for ultrasound imaging; U.S. Pat. No. 5,399,665 Biodegradable polymers for cell transplantation; U.S. Pat. No. 5,356,630 Delivery system for controlled release of bioactive factors; U.S. Pat. No. 5,330,768 Controlled drug delivery using polymer/pluronic blends; U.S. Pat. No. 5,286,763 Bioerodible polymers for drug delivery in bone; U.S. Pat. No. 5,149,543 Tonically cross-linked polymeric microcapsules; U.S. Pat. No. 5,128,420 Method of making hydroxamic acid polymers from primary amide polymers; U.S. Pat. No. 5,122,367 Polyanhydride bioerodible controlled release implants for administration of stabilized growth hormone; U.S. Pat. No. 5,100,668 Controlled release systems containing heparin and growth factors; U.S. Pat. No. 5,019,379 Unsaturated polyanhydrides; U.S. Pat. No. 5,010,167 Poly(amide-and imide-co-anhydride) for biological application; U.S. Pat. No. 4,948,587 Ultrasound enhancement of transbuccal drug delivery; U.S. Pat. No. 4,946,929 Bioerodible articles useful as implants and prostheses having predictable degradation rates; U.S. Pat. No. 4,933,431 One step preparation of poly(amide-anhydride); U.S. Pat. No. 4,933,185 System for controlled release of biologically active compounds; U.S. Pat. No. 4,921,757 System for delayed and pulsed release of biologically active substances; U.S. Pat. No. 4,916,204 Pure polyanhydride from dicarboxylic acid and coupling agent; U.S. Pat. No. 4,906,474 Bioerodible polyanhydrides for controlled drug delivery; U.S. Pat. No. 4,900,556 System for delayed and pulsed release of biologically active substances; U.S. Pat. No. 4,898,734 Polymer composite for controlled release or membrane formation; U.S. Pat. No. 4,891,225 Bioerodible polyanhydrides for controlled drug delivery; U.S. Pat. No. 4,888,176 Controlled drug delivery high molecular weight polyanhydrides; U.S. Pat. No. 4,886,870 Bioerodible articles useful as implants and prostheses having predictable degradation rates; U.S. Pat. No. 4,863,735 Biodegradable polymeric drug delivery system with adjuvant activity; U.S. Pat. No. 4,863,611 Extracorporeal reactors containing immobilized species; U.S. Pat. No. 4,861,627 Preparation of multiwall polymeric microcapsules; U.S. Pat. No. 4,857,311 Polyanhydrides with improved hydrolytic degradation properties; U.S. Pat. No. 4,846,786 Bioreactor containing suspended, immobilized species; U.S. Pat. No. 4,806,621 Biocompatible, bioerodible, hydrophobic, implantable polyimino carbonate article; U.S. Pat. No. 4,789,724 Preparation of anhydride copolymers; U.S. Pat. No. 4,780,212 Ultrasound enhancement of membrane permeability; U.S. Pat. No. 4,779,806 Ultrasonically modulated polymeric devices for delivering compositions; U.S. Pat. No. 4,767,402 Ultrasound enhancement of transdermal drug delivery; U.S. Pat. No. 4,757,128 High molecular weight polyanhydride and preparation thereof; U.S. Pat. No. 4,657,543 Ultrasonically modulated polymeric devices for delivering compositions; U.S. Pat. No. 4,638,045 Non-peptide polyamino acid bioerodible polymers; U.S. Pat. No. 4,591,496 Process for making systems for the controlled release of macromolecules.

Nonmetallic radioisotopes can conveniently be linked to the vitamin B₁₂ structure through a residue of a peptide having the following formula:

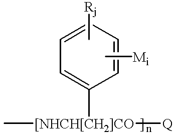

wherein each M is independently a non-metallic radionuclide; each R is independently (C₁-C₁₄)alkyl, (C₂-C₁₄)alkenyl, (C₂-C₁₄)alkynyl, (CI-CI₄)alkoxy, hydroxy, cyano, nitro, halo, trifluoromethyl, N(Rₐ)(R_{b}), (C₁-C₁₄)alkanoyl, (C₂-C₁₄)alkanoyloxy, (C₆-C₁₀)aryl or (C₃-C₈)cycloalkyl wherein Rₐ and R_{b} are each independently H or (C₁-C₁₄)alkyl; P; Q is H, (C₁-C₁₄)alkyl or a suitable carboxy protecting group; n is 2 to about 20; I is 1-5, j is 0-4 and I+j is≦5; or a pharmaceutically acceptable salt thereof. Specifically, i can be 1, j can be 0, M can be a positron emitter such as Fluorine-18, Bromine-76, Iodine-124 or a gamma emitter such as Iodine-123 or Iodine-131 and n can be about 6 to about 12.

The above discussion has demonstrated how the various variables associated with the cobalamin conjugates of the present invention can be independently varied to more particularly define specific classes of cobalamin conjugates encompassed by this invention. It is to be understood that the modification of one variable can be made independently of the modification of any other variable. Moreover, any number of embodiments can be defined by modifying two or more of the variables in such embodiments. A few of such embodiments are described below for purposes of exemplification.

Subembodiment 1: X is 5′-deoxyadenosyl; M is a divalent heterocycle wherein the radical positions can be within the ring or a substituent to the ring such that at least one radical is on a heteroatom to form a dative bond with cobalt, optionally substituted by L-T; K is O, S, NJ¹, CR¹⁰⁰R¹⁰¹ or C(R¹⁰⁰)V⁸Z⁸; E is O or S; G¹ is hydrogen, alkyl, acyl, silyl, mono-, di- or tri-phosphate or L-T; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z¹, Z², Z³, Z⁴, Z⁵, Z⁷ and Z⁸, M or G¹ comprises a radionuclide; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 2: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z¹, Z², Z³, Z⁴, Z⁵, Z⁷ and Z⁸, M or G¹ comprises a radionuclide; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 3: X is 5′-deoxyadenosyl; M is a divalent heterocycle wherein the radical positions can be within the ring or a substituent to the ring such that at least one radical is on a heteroatom to form a dative bond with cobalt, optionally substituted by L-T; K is O, S, NJ¹, CR¹⁰⁰R¹⁰¹ or C(R¹⁰⁰)V⁸Z⁸; E is O or S; G¹ is hydrogen, alkyl, acyl, silyl, mono-, di- or tri-phosphate or L-T; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 4: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z¹, Z², Z³, Z⁴, Z⁵, Z⁷, Z⁸, M and G¹ comprises a radionuclide; J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or ammo.

Subembodiment 5: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, CI- carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 6: X is hydrogen, cyano, amino, amido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 7: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z¹, Z², Z³, Z⁴, Z⁵, Z⁷, Z⁸, M and G¹ comprises a radionuclide; J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 8: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ independently are hydrogen, lower alkyl, lower alkenyl, lower alkynyl, lower cycloalkyl, heterocyclic, lower alkoxy, azido, amino, lower alkylamino, halogen, thiol, SO₂, SO₃, carboxylic acid, CI-₆ carboxyl, hydroxyl, nitro, cyano, oxime or hydrazine; R¹³ and R¹⁴ optionally can come together to form a double bond; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 9: X is hydrogen, cyano, amino, amnido, hydroxyl, 5′-deoxyadenosyl, L-T, alkyl, alkenyl, alkynyl, cycloalkyl, aryl, aralkyl, heterocycle or heteroaryl or alkylheteroaryl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ all retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiment 10: X is 5′-deoxyadenosyl; M, K, E and G¹ retain their natural vitamin B₁₂ configuration; Y¹, Y², Y³, Y⁴, Y⁵, Y⁶ and Y⁷ independently are O, S or NJ²; V¹, V², V³, V⁴, V⁵, V⁶, V⁷ and V⁸ independently are O, S or NJ³; CR¹⁰²R¹⁰³ or a direct bond; Z¹, Z², Z³, Z⁴, Z⁵, Z⁷ and Z⁸ independently are R¹⁰⁴, L-T or L-T′; each L is independently a direct bond or the residue of a multivalent moiety that does not significantly impair the ability of the compound to bind transcobalamin or intrinsic factor proteins; each L is independently a direct bond or the residue of a multivalent moiety that does not significantly impair the ability of the compound to bind transcobalamin or intrinsic factor proteins; each T or T′ independently comprises the residue of one or more radionuclides; at least one of Z², Z⁴ or Z⁵ comprises a radionuclide, the remaining Z moieties retaining their natural vitamin B₁₂ configuration; J¹, J² and J³ independently are hydrogen, alkyl, alkenyl, alkynyl, alkaryl, cycloalkyl, aryl, cycloaryl, heterocycle, heteroaryl, hydroxyl, alkoxy or amine; R¹, R², R³, R⁴, R⁵, R⁶, R⁷, R⁸, R⁹, R¹⁰, R¹¹, R¹², R¹³, R¹⁴ and R¹⁵ all retain their natural vitamin B₁₂ configuration; and R¹⁰⁰, R¹⁰¹, R¹⁰², R¹⁰³ and R¹⁰⁴ are independently hydrogen, alkyl, alkenyl, alkynyl, hydroxyl, alkoxy, cyano, azido, halogen, nitro, SO₂, SO₃, thioalkyl or amino.

Subembodiments 11-20: Any one of subembodiments 1-10, wherein the linker has a substantially constant molecular weight.

Subembodiments 21-30: Any one of subembodiments 1-10, wherein the linker is a polyamine of the following chemical structure: NR′(alkylene-NR′)ₙalkyleneNR′, wherein n is from 1 to 20, the carbon length of alkylene can vary within the n units and each R′ is independently hydrogen, lower alkyl or T.

Subembodiments 31-40: Any one of subembodiments 1-10, wherein the linker is spermine, spermidine, decamethylene tetraamine or pentamethylene hexamine.

VI. Detectable Radionuclides

As used herein, a “detectable radionuclide” is any suitable radionuclide (i.e. radioisotope) capable of being detected in a diagnostic procedure in vivo or in vitro. Suitable detectable radionuclides include metallic radionuclides (i.e. metallic radioisotopes) and non-metallic radionuclides (i.e. non-metallic radioisotopes).

Suitable metallic radionuclides (i.e. metallic radioisotopes or metallic paramagnetic ions) include Antimony-124, Antimony-125, Arsenic-74, Barium-103, Barium-140, Beryllium-7, Bismuth-206, Bismuth-207, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-67, Erbium-169, Europium-152, Gallium-64, Gallium-68, Gadolinium-153, Gadolinium-157 Gold-195, Gold-199, Hafnium-175, Hafnium-175-181, Holmium-166, Indium-110, Indium-111, Iridium-192, Iron-55, Iron-59, Krypton-85, Lead-210, Lutetium-177, Manganese-54, Mercury-197, Mercury-203, Molybdenum-99, Neodymium-147, Neptunium-237, Nickel-63, Niobium-95, Osmium-185+191, Palladium- 103, Platinum-195m, Praseodymium-143, Promethium- 147, Protactinium-233, Radium-226, Rhenium-186, Rhenium-188, Rubidium-86, Ruthenium-103, Ruthenium-106, Scandium-44, Scandium-46, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65 and Zirconium-95.

The compounds of the invention can also comprise one or more (e.g. 1, 2, 3 or 4) non-metallic radionuclide which can be directly linked to a residue of the compound of formula I at any synthetically feasible site or can be linked to a residue of the compound of formula I, by a linker, at any synthetically feasible site. Suitable linkers are described herein. In addition, suitable points of attachment of a compound of formula I for the non-metallic radionuclide, either directly or by a linker, are also described herein. The invention also provides compounds having more than one non-metallic radionuclide attached to a compound of formula I, either directly or by a linker.

Specifically, the non-metallic radionuclide can be a non-metallic paramagnetic atom (e.g. Fluorine-19); or non-metallic positron emitting radionuclide (e.g. Carbon-11, Fluorine-18, Iodine-12 or Bromine-76) or a nonmetallic gamma emitting radionuclide such as Iodine-123 or Iodine-131. Fluorine-19 is a suitable non-metallic paramagnetic for use the compounds of the present invention in part because there is typically little or no background noise associated with the diagnostic use of fluorine in the body of a mammal (e.g. human).

VII. Chelating Group

Chelating groups can be used to link radionuclides to the cobalamin conjugate of the present invention. Any suitable chelating group can be employed. Suitable chelating groups include those disclosed in U.S. Pat. Number 5,739,313. Other suitable chelating groups are the thiazoline derivatives disclosed in U.S. Pat. No. 6,083,966, the pyridinones disclosed in U.S. Pat. No. 5,892,029 and the catecholaurates disclosed in U.S. Pat. No. 5,514,695.

As used herein, a “therapeutic chelating group” is a chelating group comprising a metallic or nonmetallic radionuclide that possesses therapeutic efficacy against infections in vivo or in vitro. Any suitable chelating group can be employed.

Specifically, the therapeutic chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising a metallic radionuclide. More specifically, the therapeutic chelating group can be any of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising Rhenium-186 or Rhenium-188.

In one embodiment, the chelating group can be NTA, HEDTA, DCTA, RP414, MDP, DOTATOC, CDTA, HYNIC, EDTA, DTPA, TETA, DOTA, DOTMP, DCTA, 15N4, 9N3, 12N3 or MAG3 (or another suitable polyamino acid chelator), which are described herein below or a phosphonate chelator (e.g. EDMT). In a preferred embodiment, the chelating group is DTPA.

DTPA is diethylenetriaminepentaacetic acid; TETA is 1,4,8,11 -tetraaza-cyclo-tetradecane-N,N′,N″,N′″-tetraacetic acid; DOTA is 1,4,7,10-tetraaza-cyclododecane-N,N′,N″,N′″-tetraacetic acid; 15N4 is 1,4,8,12-tetraazacyclo-pentadecane-N,N′,N″,N′″-tetra-acetic acid; 9N3 is 1,4,7-triazacyclononane-N,N′,N″-triacetic acid; 12N3 is 1,5,9-triazacyclo-dodecane-N,N′,N″-triacetic acid; polyaminoacid chelators, such as MAG3 is (N-(N-(N-((benzoylthio)acetyl)glycyl)glycyl)glycine); and DCTA is a cyclohexane-based metal chelator of the formula

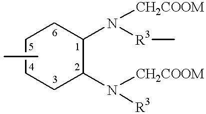

wherein R³ may by (C₁-C₄)alkyl or CH₂CO₂—, which may be attached through positions 4 or 5 or through the group R³ and which carries from 1 to 4 detectable metal or nonmetal cations (M), monovalent cations or the alkaline earth metals. Thus, with metals of oxidation state +1, each individual cyclohexane-based molecule may carry up to 4 metal cations (where both R³ groups are CH₂COOM). As is more likely, with higher oxidation states, the number of metals will decrease to 2 or even 1 per cyclohexane skeleton. This formula is not intended to limit the molecule to any specific stereochemistry.

NTA, HEDTA and DCTA are disclosed in Poster Sessions, Proceedings of the 46th Annual Meeting, J. Nuc. Med., p. 316, No. 1386. RP414 is disclosed in Scientific Papers, Proceedings of the 46th Annual Meeting, J. Nuc. Med., p. 123, No. 499. MDP is disclosed in Scientific Papers, Proceedings of the 46th Annual Meeting, J. Nuc. Med., p. 102, No. 413. DOTATOC is disclosed in Scientific Papers, Proceedings of the 46th Annual Meeting, J. Nuc. Med., p. 102, No. 414 and Scientific Papers, Proceedings of the 46th Annual Meeting, J. Nuc. Med., p. 103, No. 415. CDTA is disclosed in Poster Sessions, Proceedings of the 46th Annual Meeting, J. Nuc. Med., p. 318, No. 1396. HYNIC is disclosed in Poster Sessions, Proceedings of the 46th Annual Meeting, J. Nuc. Med., p. 319, No. 1398.

Bifunctional chelators (i.e. chelating groups) based on macrocyclic ligands in which conjugation is via an activated arm attached to the carbon backbone of the ligand can also be employed as a chelating group, as described by M. Moi et al., J. Amer. Chem., Soc., 49, 2639 (1989) (2-p-nitrobenzyl-1,4,7,10-tetraazacyclododecane-N,N′,N″,N′″-tetraacetic acid); S. V. Deshpande et al., J. Nucl. Med., 31, 473 (1990); G. Kuser et al., Bioconj. Chem., 1, 345 (1990); C. J. Broan et al., J. C. S. Chem. Comm., 23, 1739 (1990); and C. J. Anderson et al., J. Nucl. Med. 36, 850 (1995) (6-bromoacetamido-benzyl-1,4,8,11-tetraazacyclotetadecane-N,N′,N″,N′″-tetraacetic acid (BAT)).

In addition, the chelator or chelating group can be any of the chelating groups disclosed in Scientific Papers, Proceedings of the 46th Annual Meeting, J. Nuc. Med., Wednesday, Jun. 9, 1999, p. 124, No. 500.

Specifically, the chelating group can be any one of the carbonyl complexes disclosed in Waibel et al., Nature Biotechnology, 897-901, Vol. 17, September 1999; or Sattelberger et al., Nature Biotechnology, 849-850, Vol. 17, September 1999.

Specifically, the detectable chelating group can be any one of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising a metallic radionuclide. More specifically, the detectable chelating group can be any one of the carbonyl complexes disclosed in Waibel et al., Nature Biotechnology, 897-901, Vol. 17, September 1999; or Sattelberger et al., Nature Biotechnology, 849-850, Vol. 17, September 1999, further comprising Technetium-99m, Rhenium-186 or Rhenium-188.

VIII. Antibiotics

As used herein, an “antibiotic agent” is any compound having activity against either Gram-positive or Gram-negative organisms (i.e. inhibits the growth or destroys the development of either Gram-positive or Gram-negative organisms). Stedman's Medical Dictionary., Illustrated, (25^{th} Ed.), Williams & Wilkins: Baltimore (1990) and Mosby's Medical, Nursing & Allied Health Dictionary, (5^{th} Ed.), Mosby: St. Louis (1998).

Suitable antibiotic agents are disclosed, e.g. in Physician's Desk 30 Reference (PDR), Medical Economics Company (Montvale, N.J.), (53^{rd} Ed.), 1999; Mayo Medical Center Formulary, Unabridged Version, Mayo Clinic (Rochester, Minn.), January 1998; Merck Index An Encyclopedia of Chemicals, Drugs and Biologicals, (11^{th} Ed.), Merck & Co., Inc. (Rahway, N.J.), 1989; University of Wisconsin Antimicrobial Use Guide, htt ://www.medsch.wisc.edu/clinsci/ 5amcg/amcg.html; Introduction on the Use of the Antibiotics Guideline, of Specific Antibiotic Classes, Thomas Jefferson University, http://ieffiine.tju.edu/CWIS/OAC/antibiotics guide/ intro.html; and references cited therein.

It is appreciated that those skilled in the art understand that the antibiotic useful in the present invention is the biologically active compound present in any of the antibiotic drugs disclosed above. For example, Azactam (aztreonamn) is typically available as an injectable solution. The antibiotic agent, however, is (z)-2-[[[(2-amino-4-thiazolyl) [[(2S,-3S)-2-methyl-4-oxo- 1 -sulfo-3-azetidinyl]carbamoyl]methylene]amino]oxy]-2-methylpropionic acid. Physician's Desk Reference (PDR), Medical Economics Company (Montvale. N.J.), (53^{rd} Ed.), pp. 820-823. 1999.

As used herein, a “residue of an antibiotic” is a radical of an antibiotic having one or more open valences. Any synthetically feasible atom or atoms of the antibiotic can be removed to provide the open valence, provided activity against either Gram-positive or Gram-negative organisms is substantially retained when the radical is attached, either directly or via a linker, to a residue of a compound of formula I or provided the compound, upon being linked directly or by a linker to a detectable radionuclide or paramagnetic metal atom, can effectively image the infectious disease. Based on the linkage that is desired, one skilled in the art can select suitably fanctionalized starting materials that can be derived from an antibiotic using procedures that are known in the art.

Any of the antibiotics listed in the Background, any listed below or any other such agent known or discovered to exhibit antimicrobial effect that can be more effectively delivered by conjugation to a TC- or IF-binding agent can be used in accordance with this invention.

In an alternative embodiment, any of the antibiotics listed in the Background, listed below or any other such known agents can be used in combination with a TC- or IF-binding agent/antibiotic or imaging agent to achieve a combination therapeutic effect.

The antibiotic can be bound through a covalent bond, a dative bond, a coordination bond, complexation (such as found in a bound antibody/epitope) or ionic bond. Covalent bonding is preferred over ionic bonding, however, a tightly held ionic bond may be suitable. Below are nonlimiting examples of how agents can be attached to carriers. Other routine means are known to those skilled in the art and are assumed included within the scope of the invention.

Free amine or amide

The following are examples of antibiotics that contain an amine or an amide group and thus can be linked to the TC- or IF-binding agent through that functional moiety, using standard chemical reactions for covalent bond formation to a nitrogen atom.

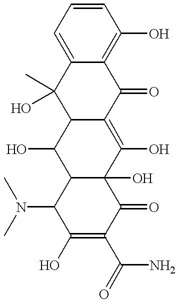

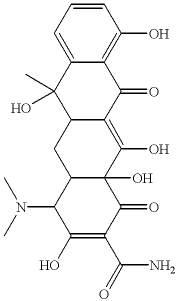

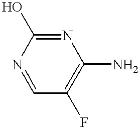

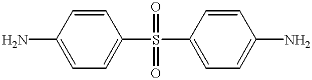

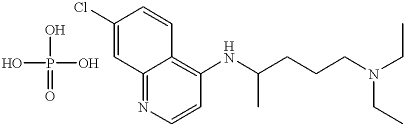

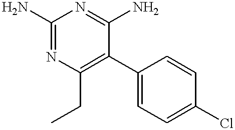

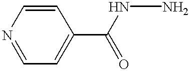

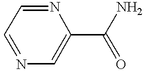

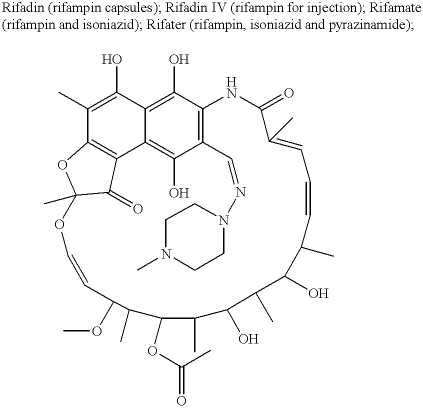

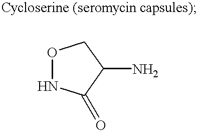

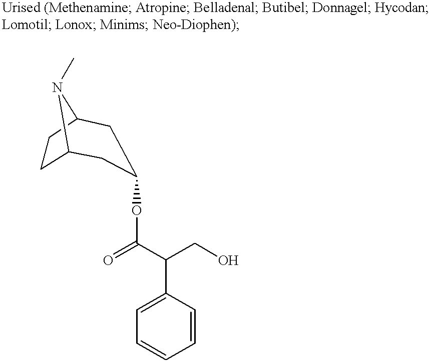

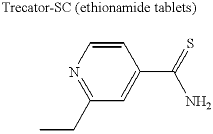

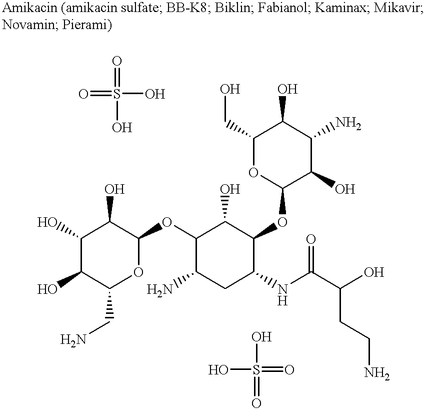

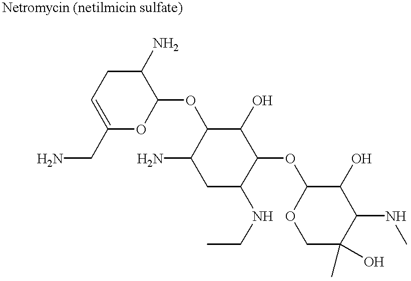

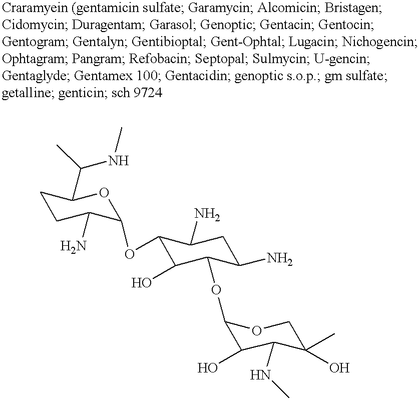

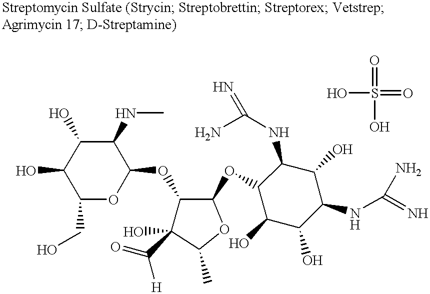

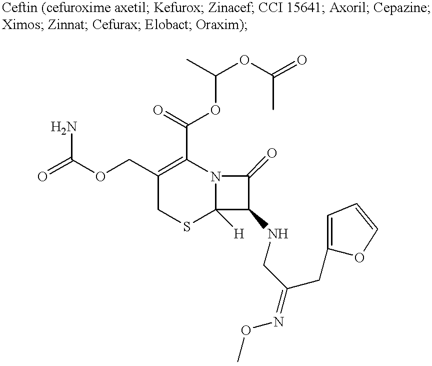

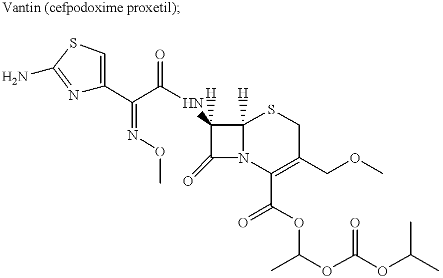

Sulfisoxazole acetyl (Gantrisin; Sulfafurazole; SK-Soxazole; urogan; urisoxin; US-67; entusil; sulfoxol; roxosul; SOXO; soxisol);

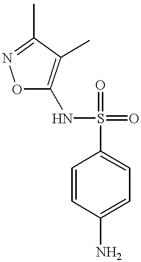

Vancocin HCl (vancomycin hydrochloride)

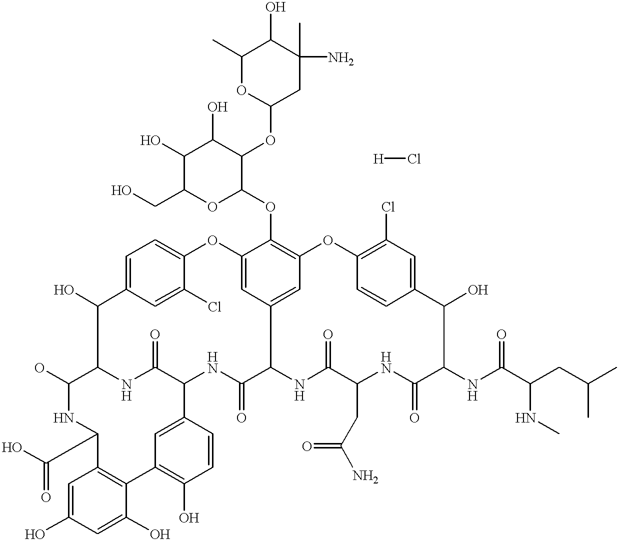

Symmetrel Syrup (amantadine HCl);

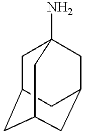

Flumadine (rimantadine HCl);

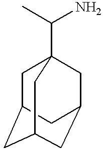

Daraprim (pyrimethamine);

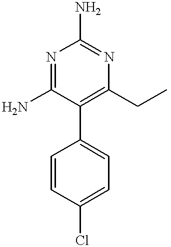

Mepron (atovaquone);

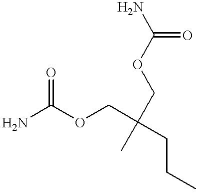

Bactrim (sulfamethoxazole); Bactrim DS (Irimethoprim and sulfamethoxazole double strength);

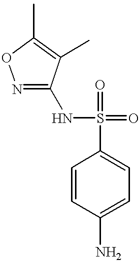

Trimethoprim (Proloprim; Trimpex);

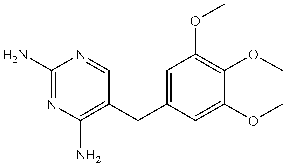

Sulfapyridine,

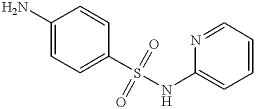

Furadantin (nitrofurantoin; Macrodantin);

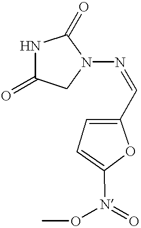

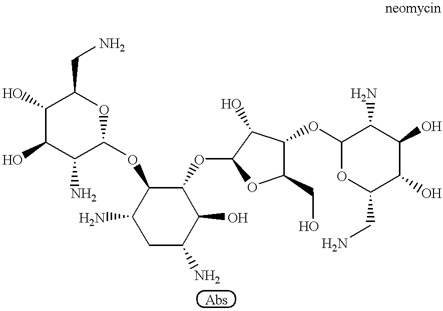

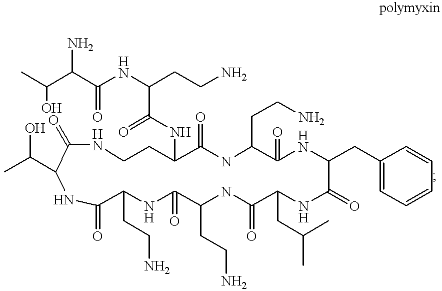

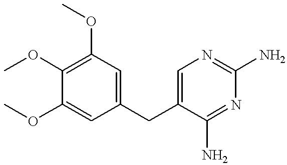

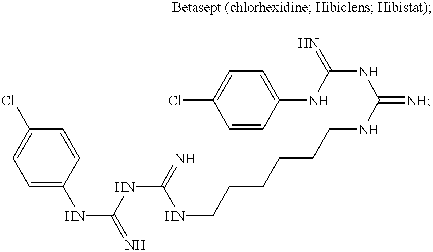

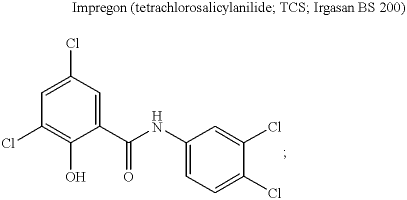

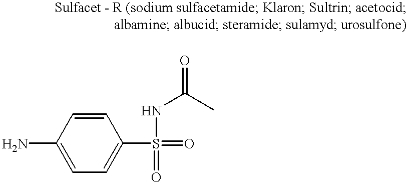

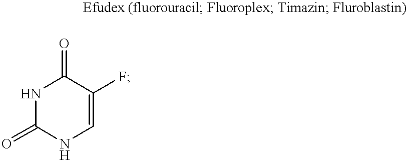

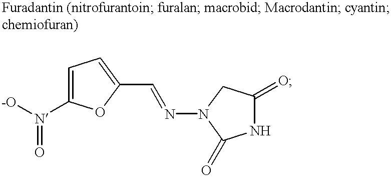

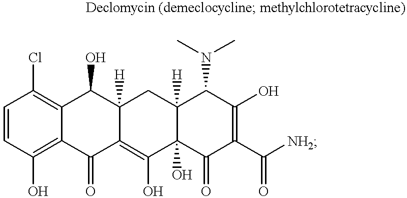

Dynacin (minocylcine HCl; Minocin; Vectrin);

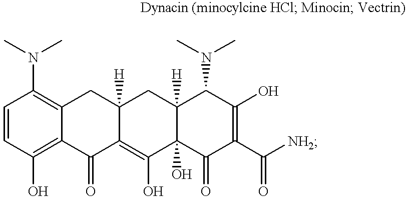

Neutrexin (trimetrexate).

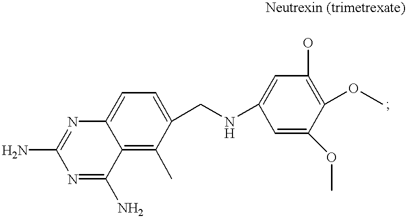

Nizoral (Ocetoconazole; ketoconazole);

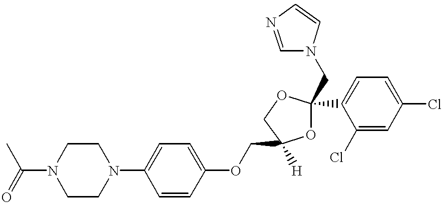

phenazopyridine;

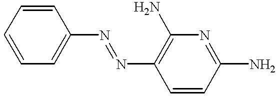

Viramune (nevirapine; BI-RG-587);

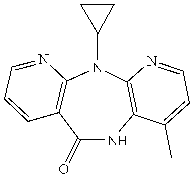

Rescriptor (delavirdine);

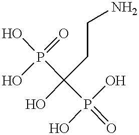

Sustiva (Efavirenz);

Invirase (saquinavir);

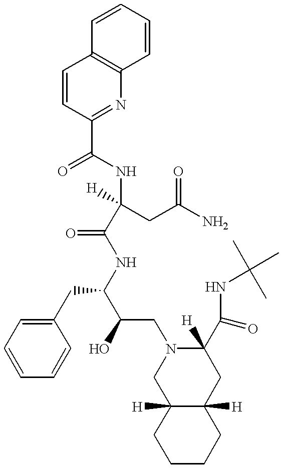

Crixivan (indinavir);

Norvir (ritonavir);

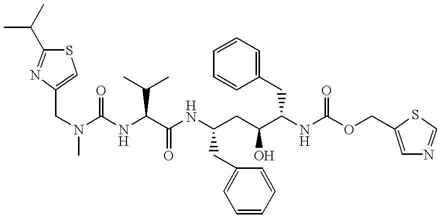

Viracept (nelfinavir);

Agenerase (Amprenavir);

Famvir (famciclovir);

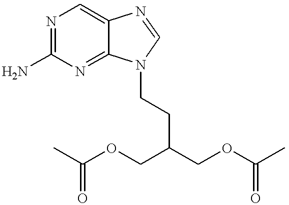

Valtrex (valacyclovir HCl);

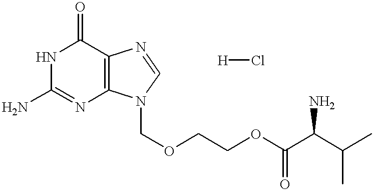

Free hydroxyl

The following are examples of antibiotics that contain an alcohol moiety and thus can be linked to the TC- or IF-binding agent through that functional moiety, using standard chemical reactions for covalent bond formation by derivatization of a hydroxyl.

TOBI (tobramycin; Nebcin; Nebramycin factor 6; 3′-Deoxykanamycin B);

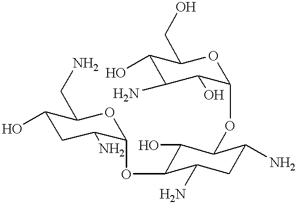

Biaxin (clarithromycin; A-56268; TE-031; 6-O-methylerythromycin; Klacid);

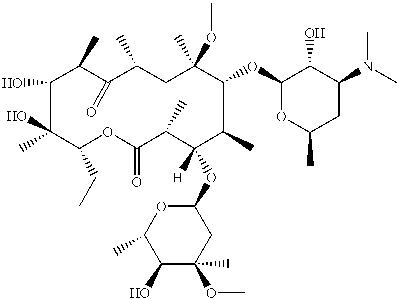

Dynabac (dirithromycin—(9S)-9-Deox-11-deoxy-9,11-[imino[(1R)-2-(2-methoxy-ethoxy)ethylidene]oxy]erythromycin;

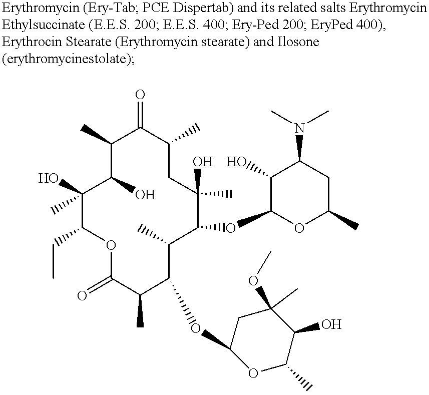

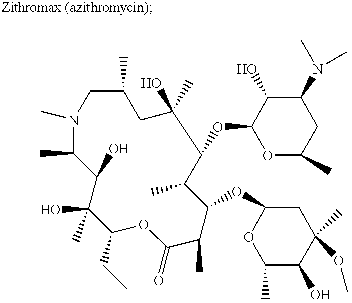

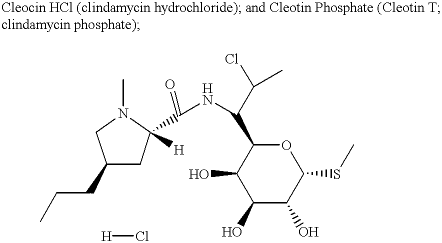

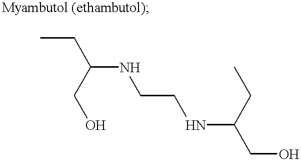

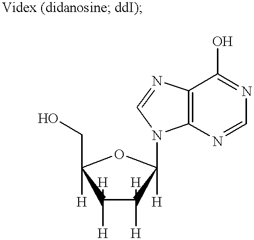

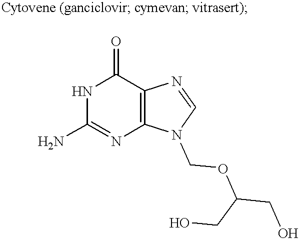

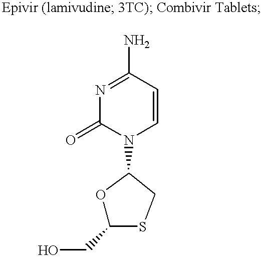

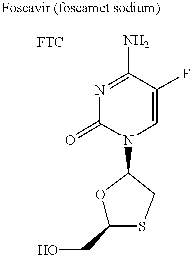

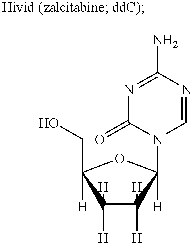

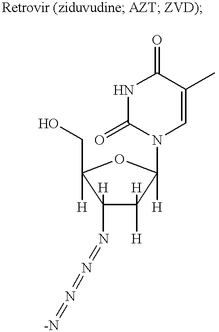

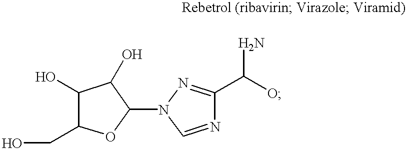

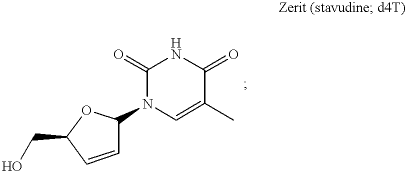

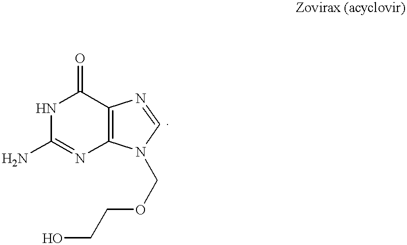

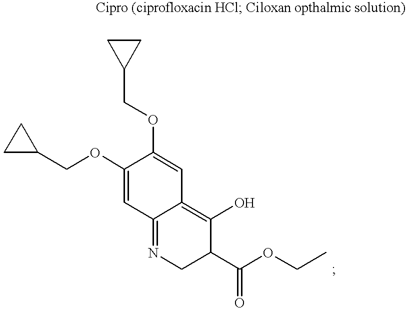

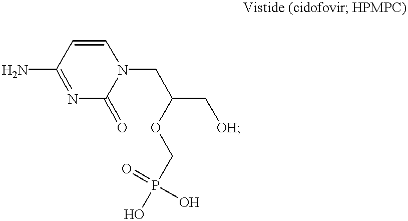

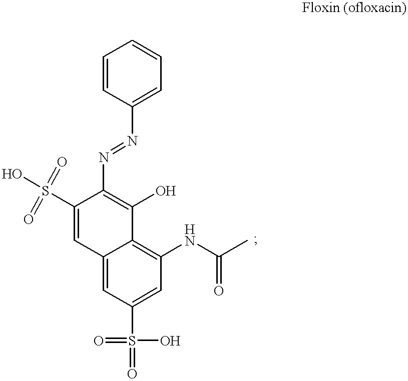

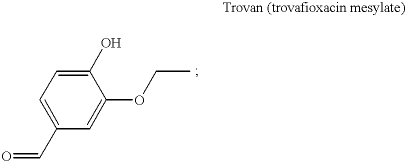

dexamethasone sodium phosphate;

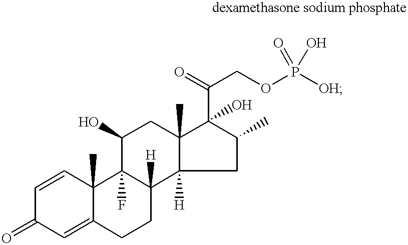

hydrocortisone (prednisolone);

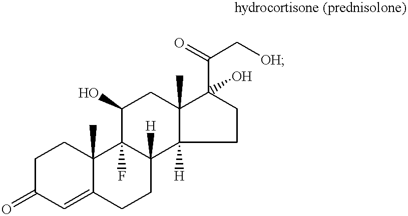

dexamethasone (opthalmic suspension and ointment)

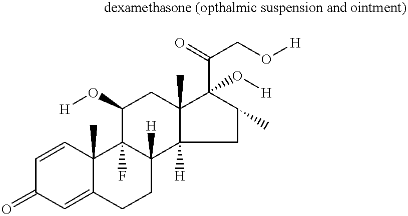

Mycostatin (nystatin cream; Nystop);

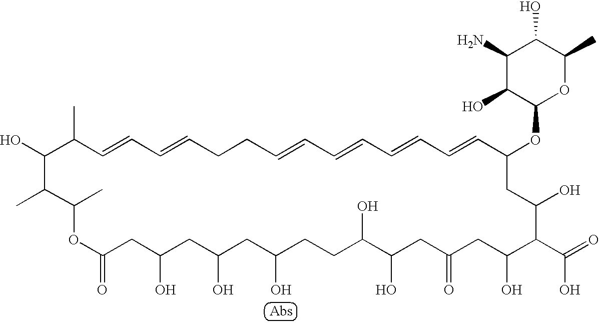

Plaquenil (hydroxychloroqnine sulfate)

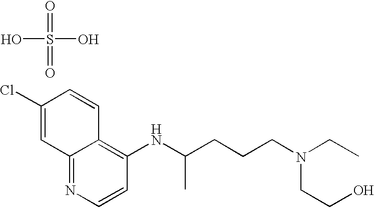

Chloromycetin (chloramphenicol opthalmic ointment);

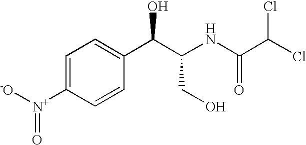

Loprox (ciclopiroxolamine);

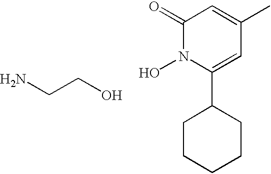

MetroCream (metronidazole; MetroGel; Noritate);

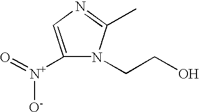

pHisoHex (hexachlorophene detergent cleanser);

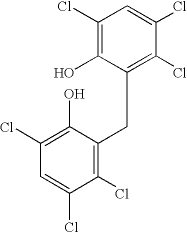

Chloromycetin opthalmic (chloramphenical; Amphicol; Cloramical; Intramyctin; Leukomycin; Anacetin; Enteromycetin; Levomycetin; Myscel; Mycinol);

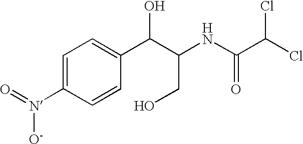

Denavir (penciclovir creamP;

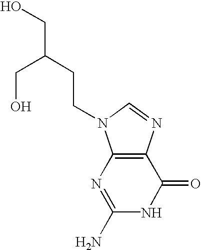

Mycobutin (rifabutin capsules);

[chemical structure]

Free Carboxylic Acid

The following are examples of antibiotics that contain a carboxylic acid moiety and thus can be linked to the TC- or IF-binding agent through that functional moiety, using standard chemical reactions for covalent bond formation by derivatization of a carboxylic acid.

PASER (aminosalicylic acid; Deapasil);

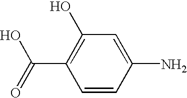

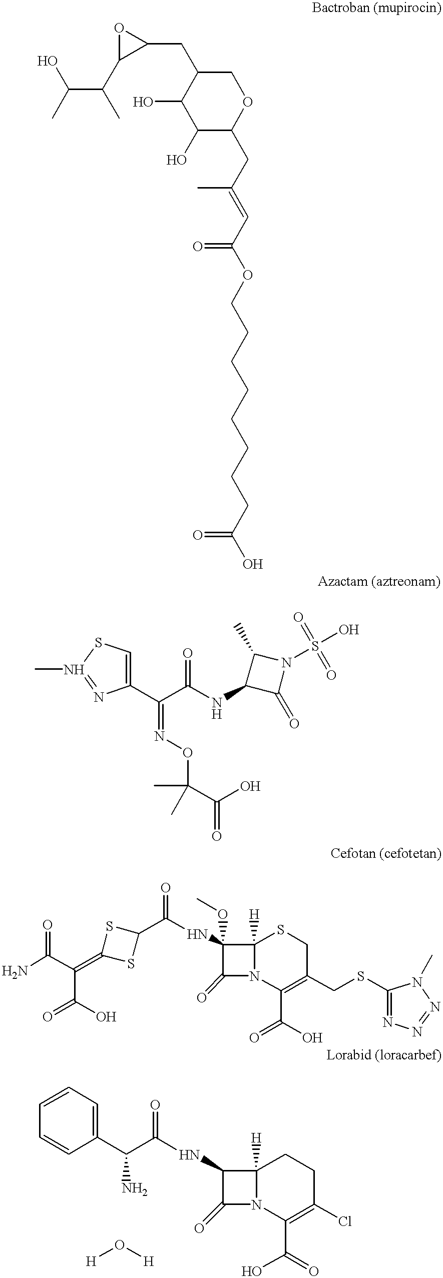

Cedax (ceffibuten)—(+)-(6R,7R)-7-[(Z)-2-(2-(2-amnino-4-thiazoly)-4-carboxycroton-amido]-8-oxo-5-thia-1-azabicyclo[4.2.0]oct-2-ene-2-carboxylic acid, dihydrate;

Cefizox (ceffizoxime sodium)—[6R-[6α,7β(Z)]]-7-[[2,3-dihydro-2-imino-4-thiazolyl)-(methoxyamino)acetyl]amino]-8-oxo-5-thia-1-azabicyclo[4.2.0]oct-2-ene-2-carboxyolic acid;

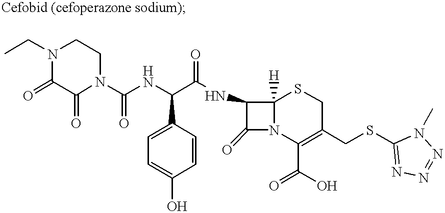

Cefzil (cefprozil)—(6R,7R)-7-[R-2-amino-2-(p-hydroxyphenyl)acetamido]-8-oxo-3-propeenyl-5-thia-1-azabicyclo[4.2.0]oct-2-ene-2-carboxylic acid monohydrate

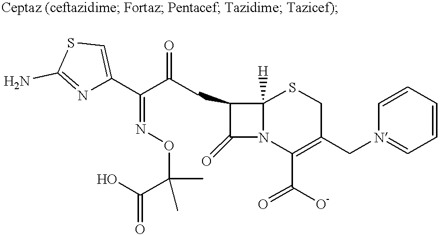

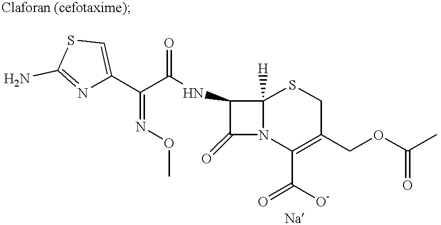

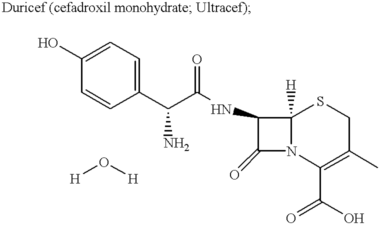

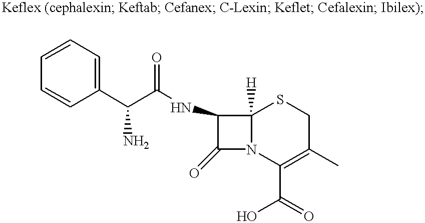

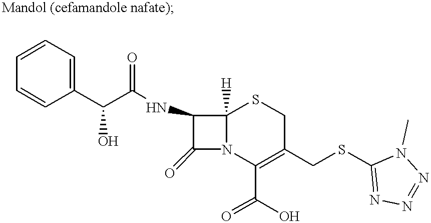

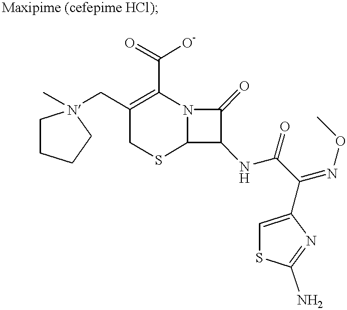

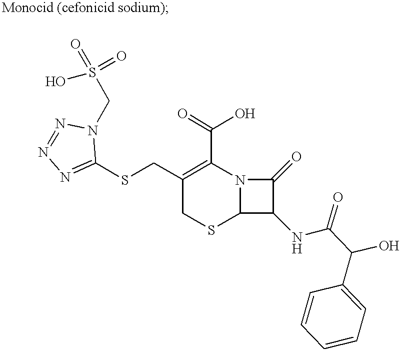

Omnicef (cefdinir)—[6R-[6α,7β(Z)]]-7-[[(2-amino-4-thiazolyl)(hydroxyimino)-acetyl]amino]-3-ethenyl-8-oxo-5-thia-1-azabicyclo[4.2.0]-oct-2-ene-2-carboxylic acid;

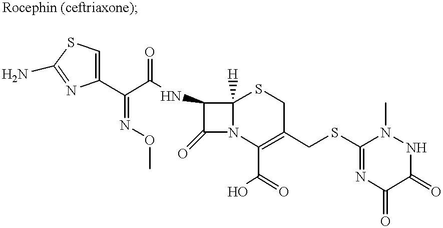

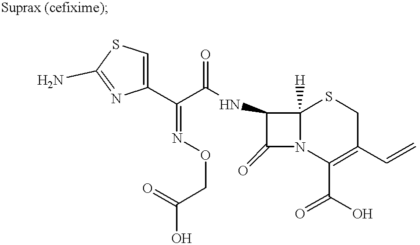

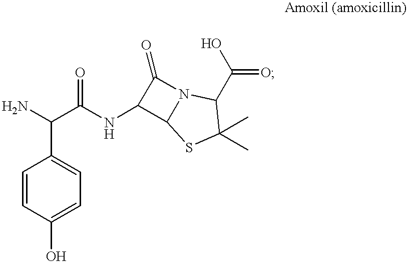

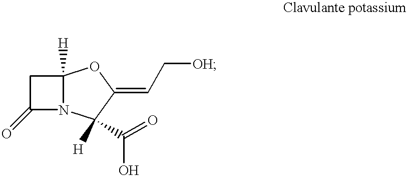

Pfizerpen (penicillin G potassium; Benzylpenicillin) and its related Bicillin C-R 900/300 (Penicillin G benzathine and Penicillin G procaine suspension; Bicillin C-R; Bicillin L-A);

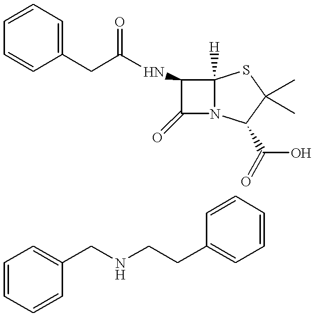

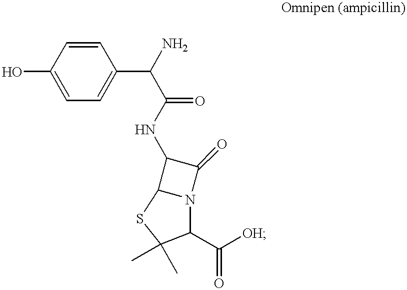

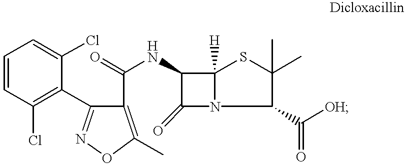

Abelcet (amphotericin B lipid complex); AmBisome (amphotericin B); Amphotec (amphotericin B cholesterol sulfatecomplex);

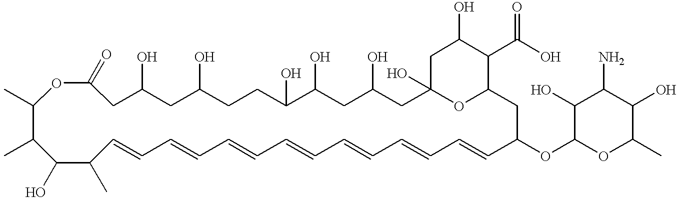

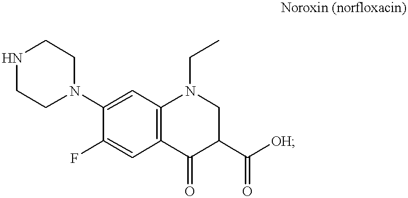

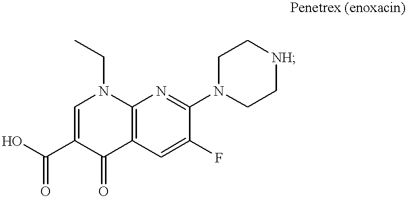

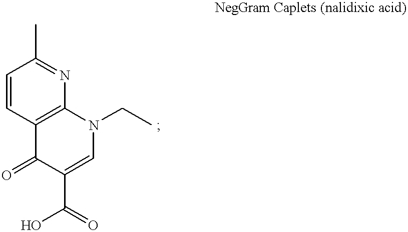

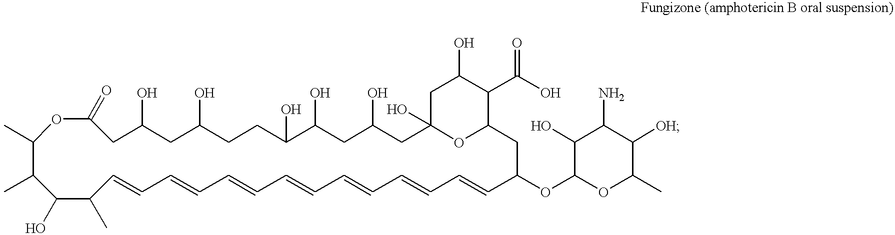

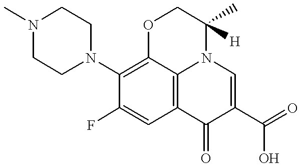

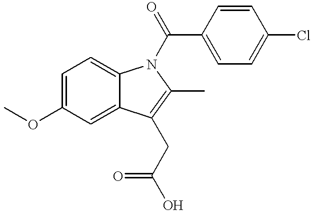

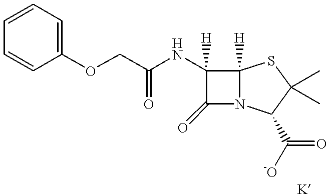

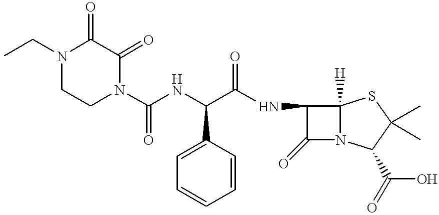

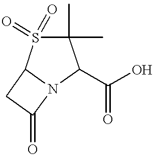

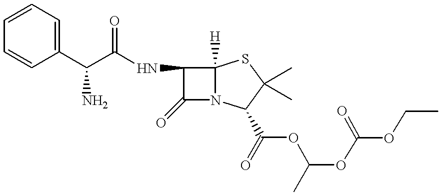

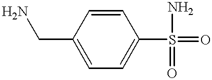

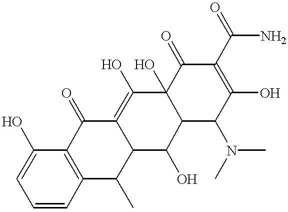

Zagam (sparfloxacin)—(cis)-5-Amino-1-cyclopropyl-7-(3,5-dimethyl-1-piperazinyl)-6,8-difluoro-1,4-dihydro-4-oxo-3-quinolinecarboxylic acid

Miscellaneous

The following antibiotics do not have readily available functional groups to derivatize for covalent attachment to the TC- or IF-binding agent or linker, but can be attached through a suitable ionic bond with close salt formation, wherein the carrier or linker contains an appropriate counterion.

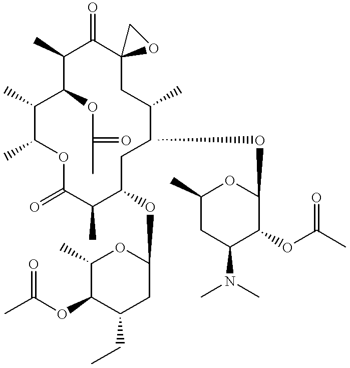

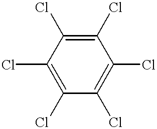

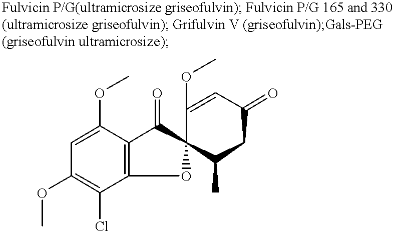

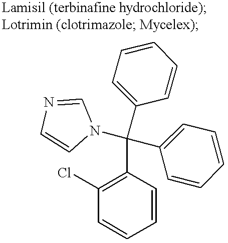

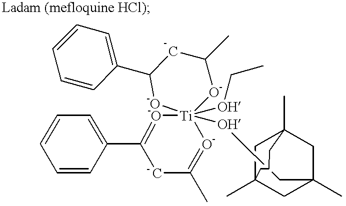

Diflucan (fluconazole);

Sporanox (itraconazole).

Alferon N (interferon alfa-n3); Intron A (interferon alfa-2b);

Flagyl 375 (metronidazole); Flagyl ER Tablets (metronidazole); Flagyl I.V. (metronidazole);

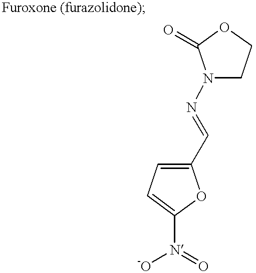

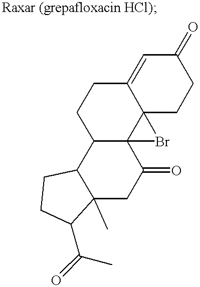

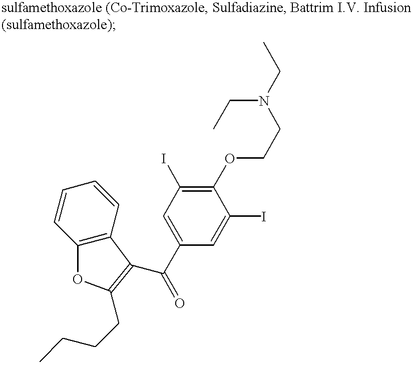

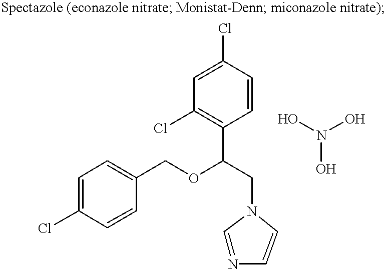

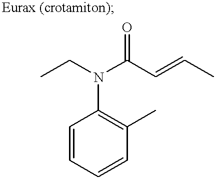

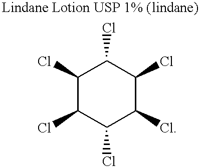

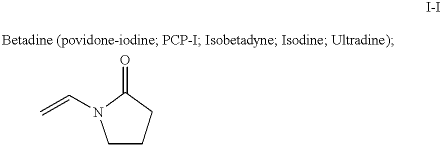

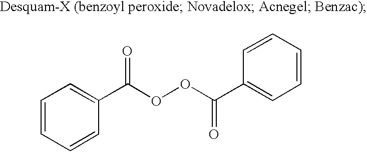

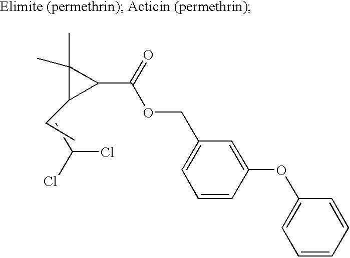

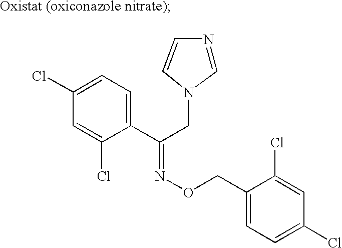

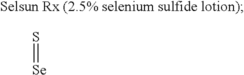

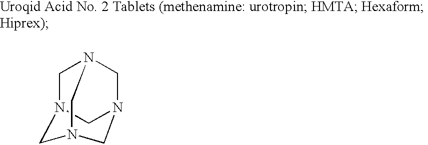

IX. Synthetic Techniques

Various synthetic techniques are known for preparing the compounds of the present invention. For example, compounds wherein the residue of an imaging agent is directly linked to the 6-position of a compound of formula I (i.e. in which X is L-T and L is a direct bond) can be prepared by reducing a corresponding Co (III) compound of formula I to form a nucleophilic Co (I) compound and treating this Co (I) compound with a residue of a imaging agent (or a derivative thereof) comprising a suitable leaving group, such as a halide. Similarly, compounds wherein X is L-T and L is other than a direct bond can be prepared by preparing a nucleophilic Co (I) species as described herein above and reacting it with a linker comprising a suitable leaving group, such as a halide. Peptides and amino acids can be attached to the 6-position by reducing a corresponding Co (III) compound of formula I to form a nucleophilic Co (I) compound and treating the Co (I) compound with a suitable alkylating agent comprising an amino acid or peptide.

Coupling of L-T to the ribose moiety at K or G¹ may be accomplished by activating the natural OH at either K or G¹ with a suitable reagent such as succinic anhydride, to yield a reactive group such as a carboxylate. This technique is described in detail in Toraya, Bioinorg. Chem. 4:245-255, 1975.

Coupling of L-T to M can be accomplished using techniques described in detail in Jacobsen, Anal. Biochem. 113:164-171, 1981.

The residue of vitamin B₁₂ or its analog can be prepared by any suitable means known in the art. For example, a monocarboxylic acid or dicarboxylic acid of cobalamin can be prepared as disclosed in U.S. Pat. No. 5,739,313. These compounds can be prepared by the mild acid hydrolysis of cyanocobalamin, which has been shown to yield a mixture of mono-, a dicarboxylic acid and one tricarboxylic acid. These carboxylic acids are derived from the propionamide side chains designated b, d- and e-, as discussed hereinabove, which are more susceptible to hydrolysis than the amide groups on acetamide side chains a-, c- and g-. The b-, d- and e-monocarboxylic acids can be separated by column chromatography. L. Anton et al., J. Amer. Chem. Soc.,102, 2215 (1980). See, also, J B. Armitage et al., L Chem. Sot., 3349 (1953); K. Bernhauer, Biochem. Z., 344, 289 (1966); H. P. C. Hogenkamp et al., Biochemistry, 14, 3707 (1975); and L. Ellenbogen, in “Cobalamin,” Biochem. and Pathophysiol, B. Babior, ed., Wiley, N.Y. (1975) at chapter 5.

Additional compounds, intermediates and synthetic preparations thereof are disclosed, for example, in Hogenkamp, H. et al., Synthesis and Characterization of nido-Carborane-Cobalamin Conjugates, Nucl. Med. & Biol., 2000, 27, 89-92; Collins, D., et al., Tumor Imaging Via Indium 111-Labeled DTPA-Adenosylcobalamin, Mayo Clinic Proc., 1999, 74:687-691.

Compound of Formula I/Antibiotic Linkage

The invention provides a compound of formula I (FIG. 1) directly linked to one or more antibiotics, wherein X is CN, OH, CH₃, adenosyl or L-T, wherein T is preferably an antibiotic; or a pharmaceutically acceptable salt thereof.

The residue of an antibiotic can be linked to the residue of a compound of formula I through an amide (e.g. —NRC(═O)— or —C(═O)NR—), ester (e.g. —OC(═O)— or —C(═O)O—), ether (e.g. —O—), amino (e.g. —NR—), ketone (e.g. —C(═O)—), thioether (e.g. —S—), sulfinyl (e.g. —S(O)—), sulfonyl (e.g. —S(O)₂—) or a direct (e.g. C—C bond) linkage, wherein each R is independently H or (C₁-C₆)alkyl. Such a linkage can be formed from suitably functionalized starting materials using synthetic procedures that are known in the art. Based on the linkage that is desired, one skilled in the art can select suitably functional starting materials that can be derived from a residue of a compound of formula I and from a given residue of an antibiotic using procedures that are known in the art.

The residue of the antibiotic can be directly linked to any synthetically feasible position on the residue of a compound of formula I. Suitable points of attachment include, for example, the b-carboxamide, the d-carboxamide and the e-carboxamide (illustrated in FIG. 1), as well as the 6-position (the position occupied by X in FIG. 1) and the 5′-hydroxy and the 3′-hydroxy groups on the 5-membered sugar ring, although other points of attachment are possible. U.S. Pat. No. 5,739,313 discloses compounds (e.g. cyanocobalamin-b-(4-aminobutyl)amide, methylcobalamin-b-(4-aminobutyl)amide and adenosylcobalamin-b-(4-aminobutyl)amide) that are useful intermediates for the preparation of compounds of the present invention.

Compounds wherein the residue of an antibiotic is linked to the 6-position of a compound of formula I can be prepared by reducing a corresponding Co (III) compound of formula I to form a nucleophilic Co (I) compound and treating this Co (I) compound with a residue of an antibiotic (or a derivative thereof) comprising a suitable leaving group, such as a halide (e.g. a chloride).

The invention also provides compounds having more than one residue of an antibiotic or antibiotics directly linked to a compound of formula I. For example, the residue of an antibiotic can be directly linked to a residue of the b-carboxamide of the compound of formula I and a residue of another antibiotic can be directly linked to a residue of the d- or e-carboxamide of the compound of formula I. In addition, the residue of an antibiotic can be directly linked to the 6-position of the compound of formula I and a residue of another antibiotic can be directly linked, for example, to a residue of the b-, d- or e-carboxamide of the compound of formula I.

Compound of Formula I/Linker/Antibiotic Linkage

In addition to being directly linked to the residue of a compound of formula I, the residue of an antibiotic can also be linked to the residue of a compound of formula I by a suitable linker. The structure of the linker is not crucial, provided the resulting compound of the invention has an effective therapeutic index as an antibiotic drug and preferably can be orally administered. Suitable linkers are disclosed, for example, in U.S. Pat. No. 5,735,313; U.S. application Ser. No. 60/129,733 filed Apr. 16, 1999; U.S. application Ser. No. 60/159,874 filed Oct. 15, 1999; U.S. application Ser. No. 60/159,753 filed Oct. 15, 1999; U.S. application Ser. No. 60/159,873 filed Oct. 15, 1999; and references cited therein.

Suitable linkers include linkers that separate the residue of a compound of formula I and the residue of an antibiotic by about 5 angstroms to about 200 angstroms, inclusive, in length. Other suitable linkers include linkers that separate the residue of a compound of formula I and the residue of an antibiotic by about 5 angstroms to about 100 angstroms, inclusive, in length, as well as linkers that separate the residue of a compound of formula I and the residue of an antibiotic by about 5 angstroms to about 50 angstroms or by about 5 angstroms to about 25 angstroms, inclusive, in length.

The linker can be linked to any synthetically feasible position on the residue of a compound of formula I. Suitable points of attachment include, for example, a residue of the b-carboxamide, a residue of the d-carboxamide, a residue of the e-carboxamide, the 6-position (i.e. the position occupied by X in the compound of formula I), as well as a residue of the 5′-hydroxy group and a residue of the 3′ hydroxy group on the 5-membered sugar ring, although other points of attachment are possible. Based on the linkage that is desired, one skilled in the art can select suitably functionalized starting materials that can be derived from a compound of formula I and an antibiotic using procedures that are known in the art.

The linker can conveniently be linked to the residue of a compound of formula I or to the residue of an antibiotic through an amide (e.g. —NRC(═O)— or —C(═O)NR—), ester (e.g. —OC(═O)— or —C(═O)O—), ether (e.g. —O—), ketone (e.g. —C(═O)—) thioether (e.g. —S—), sulfinyl (e.g. —S(O)—), sulfonyl (e.g. —S(O)₂—), amino (e.g. —NR—) or a direct (e.g. C—C) linkage, wherein each R is independently H or (C₁-C₆)alkyl. The linkage can be formed from suitably functionalized starting materials using synthetic procedures that are known in the art. Based on the linkage that is desired, one skilled in the art can select suitably functional starting materials that can be derived from a residue of a compound of formula I, a residue of an antibiotic and from a given linker using procedures that are known in the art.

Specifically, the linker can be a divalent radical of the formula W—A—Q wherein A is (C₁-C₂₄)alkyl, (C₂-C₂₄) alkenyl, (C₂-C₂₄)alkynyl, (C₃-C₈)cycloalkyl or (C₆-C₁₀)aryl, wherein W and Q are each independently —NRC(═O)—, —C(═O)NR—, —OC(═O)—, —C(═O)O—, —O—, —S—, —S(O)₂—, —NR—, —C(═O)— or a direct bond (i.e. W and/or Q is absent); wherein each R is independently H or (C₁-C₆)alkyl.

Specifically, the linker can be a divalent radical of the formula W—(CH₂)ₙ—Q wherein, n is between about 1 and about 20, between about 1 and about 15, between about 2 and about 10, between about 2 and about 6 or between about 4 and about 6; wherein W and Q are each independently —NRC(═O)—, —C(═O)NR—, —OC(═O)—, —C(═O)O—, —O—, —S—, —S(O)—, —S(O)₂—, —C(═O)—, —NR— or a direct bond (i.e. W and/or Q is absent); wherein each R is independently H or (C₁-C₆)alkyl.

Specifically, W and Q can each independently be —NRC(═O)—, —C(═O)NR—, —OC(═O)—, —NR—, —C(═O)O—, —O— or a direct bond (i.e. W and/or Q is absent).

Specifically, the linker is a divalent radical, i.e. 1,ω-divalent radicals formed from a peptide or an amino acid. The peptide can comprise 2 to about 25 amino acids, 2 to about 15 amino acids or 2 to about 12 amino acids.

Specifically, the peptide can be poly-L-lysine (i.e. [—NHCH[(CH₂)₄NH₂]CO—]ₘ—Q, wherein Q is H, (C₁-C₁₄) alkyl or a suitable carboxy protecting group; and wherein m is about 2 to about 25. Specifically, the poly-L-lysine can contain about 5 to about 15 residues (i.e. m is between about 5 and about 15). More specifically, the poly-L-lysine can contain about 8 to about 11 residues (i.e. m is between about 8 and about 11).

Specifically, the peptide can be poly-L-glutamic acid, poly-L-aspartic acid, poly-L-histidine, poly-L-serine, poly-L-threonine, poly-L-tyrosine, poly-L-leucine, poly-L-lysine-L-phenylalanine or poly-L-lysine-L-tyrosine.

Specifically, the linker can be prepared from 1,6-diaminohexane H₂N(CH₂)₆NH₂, 1,5-diaminopentane H₂N(CH₂)₅NH₂, 1,4-diaminobutane H₂N(CH₂)₄NH₂ or 1,3-diaminopropane H₂N(CH₂)₃NH₂.

The invention also provides compounds having more than one antibiotic attached to a compound of formula I, each through a linker. For example, the residue of an antibiotic can conveniently be linked, through a linker, to a residue of the b-carboxamide of the compound of formula I and a residue of another antibiotic can conveniently be linked, through a linker, to a residue of the d- or e-carboxamide of the compound of formula I. In addition, the residue of an antibiotic can conveniently be linked, for example, through a linker, to the 6-position of the compound of formula I and a residue of another antibiotic can conveniently be linked, through a linker, to a residue of the b-, d- or e-carboxamide of the compound of formula I.

Compounds wherein the linker is linked to the 6-position of a compound of formula I can be prepared by preparing a nucleophilic Co (I) species as described herein above and reacting it with a linker comprising a suitable leaving group, such as a halide (e.g. a chloride).

The invention also provides compounds having more than one antibiotic attached to a compound of formula I, either directly or through a linker. For example, the residue of an antibiotic can conveniently be linked, either directly or through a linker, to a residue of the b-carboxamide of the compound of formula I and a residue of another antibiotic can conveniently be linked, either directly or through a linker, to a residue of the d- or e-carboxamide of the compound of formula I. In addition, the residue of an antibiotic can conveniently be linked, for example, either directly or through a linker, to the 6-position of the compound of formula I and a residue of another antibiotic can conveniently be linked, either directly or through a linker, to a residue of the b-, d- or e-carboxamide of the compound of formula I.

Compound of Formula I/Detectable Radionuclide Linkage

The invention provides compounds wherein a residue of compound of formula I is directly linked to a detectable radionuclide (e.g. non-metallic radionuclide). A detectable radionuclide (e.g. non-metallic radionuclide) can be linked directly to any synthetically feasible position on the residue of a compound of formula I. Suitable points of attachment include, for example, the b-carboxamide, the d-carboxamide and the e-carboxamide (illustrated in FIG. 1), as well as the 6-position (the position occupied by X FIG. 1) and the 5′-hydroxy and the 3′-hydroxy groups on the 5-membered sugar ring, although other points of attachment are possible. U.S. Pat. No. 5,739,313 discloses compounds (e.g. cyanocobalamin-b-(4-aminobutyl)amide, methylcobalamin-b-(4-aminobutyl)amide and adenosylcobalamin-b-(4-aminobutyl)amide) that are useful intermediates for the preparation of compounds of the present invention.

The invention also provides compounds having more than one detectable radionuclide (e.g. non-metallic radionuclides) directly linked to a compound of formula I. For example, the detectable radionuclide (e.g. non-metallic radionuclide) can be directly linked to a residue of the b-carboxamide of the compound of formula I and another detectable radionuclide (e.g. non-metallic radionuclide) can be directly linked to a residue of the d- or e-carboxamide of the compound of formula I. In addition, the detectable radionuclide (e.g. non-metallic radionuclide) can be directly linked to the 6-position of the compound of formula I and another detectable radionuclide (e.g. non-metallic radionuclide) can be directly linked, for example, to a residue of the b-, d- or e-carboxamide of the compound of formula I.

Compound of Formula I/Linker/Detectable Radionuclide or Paramagnetic Metal Atom

When a detectable radionuclide (e.g. metallic radionuclide) or paramagnetic metal atom is linked to the residue of a compound of formula I by a suitable linker, the structure of the link is not crucial, provided it provides a compound of the invention which has an effective therapeutic and/or diagnostic index against the target cells and which will localize in or near the infectious disease.

Suitable linkers include linkers that separate the residue of a compound of formula I and the detectable radionuclide by about 5 angstroms to about 200 angstroms, inclusive, in length. Other suitable linkers include linkers that separate the residue of a compound of formula I and the detectable radionuclide by about 5 angstroms to about 100 angstroms, as well as linkers that separate the residue of a compound of formula I and the detectable radionuclide by about 5 angstroms to about 50 angstroms, or by about 5 angstroms to about 25 angstroms. Suitable linkers are disclosed, for example, in U.S. Pat. No. 5,735,313.

The linkers can conveniently be linked to the residue of a compound of formula I through an amide (e.g. —NRC(═O)NR—), ester (e.g. —OC(═O)— or —C(═O)O—), thioether (e.g. —S—), sulfinyl (e.g. —S(O)—), Sulfonyl (e.g. —S(O)₂—) or a direct (e.g. C—C bond) linkage, wherein each R. is independently H or (C₁-C₁₄)alkyl. Such a linkage can be formed from suitably functionalized starting materials using synthetic procedures that are known in the art. Based on the linkage that is desired, one skilled in the art can select suitably functional starting materials that can be derived from a residue of a compound of formula I and from a given linker using procedures that are known in the art.

The linker can be directly linked to any synthetically feasible position on the residue of a compound of formula I. Suitable points of attachment include, for example, the b-carboxamide, the d-carboxamide, ad the e-carboxamide (illustrated in FIG. 1), as well as the 6-position (the position occupied by X in FIG. 1) and the 5′-hydroxy and the 3′-hydroxy groups on the 5 membered sugar ring, although other points of attachment are possible. U.S. Pat. No. 5,739,313 discloses compound (e.g. cyanocobalamin-b-(4-aminobytyl)amide, methylcobalamin-b-(4-amninobutyl)amide and adenosylcobalamin-b-(4-aminobutyl)amide) that are useful intermediates for the preparation of compounds of the present invention.

The invention also provides compounds having more than one linker attached to a compound of formula I. For example, the linker can be linked to a residue of the b-carboxamide of the compound of formula I and another linker can be directly linked to a residue of the d-carboxamide of the compound of formula I.

Specifically, the linker can comprise about 1 to about 20 detectable radionuclides. More specifically, the linker can comprise about 1 to 10 detectable radionuclides or about 1 to about 5 detectable radionuclides.

Specifically, the linker can be a divalent radical of the formula W—A wherein A is (C₁-C₆)alkyl, (C₂-C₆)alkenyl, (C₂-C₆)alkynyl, (C₃-C₈)cycloalkyl or (C₆-C₁₀)aryl, wherein W is —NRC(═O)—, —C(═O)NR—, —OC(═O)—, —O—, —S—, —S(O)_, S(O)₂—, —NR—, —C(═O)— or a direct bond, wherein each R is independently H or (C₁-C₆)alkyl; wherein A is linked to one or more non-metallic radionuclides.

Specifically, the linker can be an amino acid or a peptide. Specifically, the peptide can be poly-L-lysine, poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine or poly-L-lysine-L-tyrosine.

Specifically, the linker can be a chelating group capable of chelating one or more detectable radionuclides (e.g. metallic radionuclides). More specifically, the linker can be a detectable chelating group.

Specifically, the chelating group can be DTPA.

The compounds disclosed herein can be prepared using procedures similar to those described in U.S. Pat. No. 5,739,313 or using procedures similar to those described herein. The residue of an antibiotic can be linked to the residue of a compound of formula I as described hereinabove. The detectable radionuclide can be linked to the residue of a compound of formula I as described hereinabove. Additional intermediates and synthetic procedures useful for preparing intermediates of the invention are disclosed, for example, in Hogenkamp, H. et al., Synthesis and Characterization of nido-Carborane-Cobalamin Conjugates, Nucl. Med. & Biol., 2000, 27, 89-92; Collins, D., et al., Tumor Imaging Via Indium 111-Labeled DTPA-Adenosylcobalamin, Mayo Clinic Proc., 1999, 74:687-691; U.S. application Ser. No. 60/129, 733 filed Apr. 16, 1999; U.S. application Ser. No. 06/159, 874 filed Oct. 15, 1999; U.S. application Ser. No. 60/159,753 filed Oct. 15, 1999; U.S. application Ser. No. 60/159,873 filed Oct. 15, 1999; U.S. Pat. No. 5,739,313; U.S. Pat. No. 6,004,533; and references cited therein.

X. Therapeutic and Diagnostic Compositions and Administrations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate and α-glycerophosphate. Suitable inorganic salts may also be formed, including, sulfate, nitrate, bicarbonate and carbonate salts.

Pharmaceutically acceptable salts may be obtained using standard procedures well known in the art, for example by reacting a sufficiently basic compound such as an amnine with a suitable acid affording a physiologically acceptable anion. Alkali metal (for example, sodium, potassium or lithium) or alkaline earth metal (for example calcium) salts of carboxylic acids can also be made.

Preferred modes of administration of the TC- or IF-binding agents and imaging agents are parenteral, intravenous, intradermal, intra-articular, intra-synovial, intrathecal, intra-arterial, intracardiac, intramuscular, subcutaneous, intraorbital, intracapsular, intraspinal, intrastemal, topical, transdermal patch, via rectal, vaginal or urethral suppository, peritoneal, percutaneous, nasal spray, surgical implant, internal surgical paint, infusion pump or via catheter. In one embodiment, the agent and carrier are administered in a slow release formulation such as an implant, bolus, microparticle, microsphere, nanoparticle or nanosphere. For standard information on pharmaceutical formulations, see Ansel, et al., Pharmaceutical Dosage Forms and Drug Delivery Systems, Sixth Edition, Williams & Wilkins (1995).

The TC- or IF-binding agents/imaging agents can, for example, be administered intravenously or intraperitoneally by infusion or injection. Solutions of the substance can be prepared in water, optionally mixed with a nontoxic surfactant. Dispersions can also be prepared in glycerol, liquid polyethylene glycols, triacetin and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the substance which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, normal saline, ethanol, a polyol (for example, glycerol, propylene glycol, liquid polyethylene glycols and the like), vegetable oils, nontoxic glyceryl esters and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, benzyl alcohol, sorbic acid, thimerosal and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the substance in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Injectable solutions are particularly advantageous for local administration of the therapeutic composition. In particular, parenchymal injection can be used to deliver the therapeutic composition directly to a tumorous growth. Intra-articular injection is a preferred alternative in cases of arthritis where the practitioner wishes to treat one or only a few (such as 2-6) joints. Additionally, the therapeutic compounds are injected directly into lesions (intra-lesion administration) in appropriate cases. Intradermal administration is an alternative for dermal lesions.

The therapeutic compound is optionally administered topically by the use of a transdermal therapeutic system (see, Barry, Dermatological Formulations, (1983) p. 181 and literature cited therein). Transdermal drug delivery (TDD) has several advantages over oral delivery. When compared to oral delivery, TDD avoids gastrointestinal drug metabolism, reduces first pass effects and provides a sustained release of drugs for up to seven days (Elias, et al. Percutaneous Absorption: Mechanisms-Methodology-Drug Delivers; Marcel Dekker, NY: 1, 1989). This method is especially useful with many therapeutic proteins that are susceptible to gastrointestinal degradation and exhibit poor gastrointestinal uptake. When compared to injections, TDD eliminates the associate pain and the possibility of infection. While such topical delivery systems have been designed largely for transdermal administration of low molecular weight drugs, by definition they are capable of percutaneous delivery. They can be readily adapted to administration of the therapeutic compounds of the invention by appropriate selection of the rate-controlling microporous membrane. Topical application can also be achieved by applying the compound of interest, in a cream, lotion, ointment or oil based carrier, directly to the skin. Typically, the concentration of therapeutic compound in a cream, lotion or oil is 1-2%.

For drug targeting to lung tissue, the therapeutic compound is formulated into a solution, suspension, aerosol or particulate dispersion appropriate for application to the pulmonary system. The therapeutic agent may be inhaled via nebulizer, inhalation capsule, inhalation aerosol, nasal solution, intratracheal as a solution via syringe or endotracheal tube as an aerosol or via as a nebulizer solution. Aerosols are prepared using an aqueous aerosol, liposomal preparation or solid particles containing the compound. A nonaqueous (e.g. fluorocarbon propellant) suspension could be used. Sonic nebulizers are preferred because they minimize exposing the therapeutic compound to shear, which can result in degradation of the compound.

Delivery of the cobalamin conjugates of the instant invention by the mucosal route also offers an attractive administration alternative. The prototype formulation for nasal solutions will contain the vitamin B₁₂ conjugate dissolved in a suitable aqueous or non-aqueous solvent such as propylene glycol, an antioxidant and aromatic oils as flavoring agents. The formulation may also contain suitable propellant(s).

For ophthalmic applications, the therapeutic compound is formulated into solutions, suspensions and ointments appropriate for use in the eye. For opthalmic formulations, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983).

Useful dosages of the compounds of formula I can be determined by comparing their in vitro activity and in vivo activity in animal models. Methods for the extrapolation of effective dosages in mice and other animals, to humans are known to the art; for example, see U.S. Pat. No. 4,938,949. The amount of the substance required for use in treatment will vary not only with the particular salt selected but also with the route of administration, the nature of the condition being treated and the age and condition of the patient and will be ultimately at the discretion of the attendant physician or clinician.

In general, however, a suitable dose for nuclear medicine (using a radioactive imaging agent) will be in the range of from about 0.1 μg/patient to about 1000 μg/patient, from about 0.5 to about 500 μg/patient or from 1 μg/patient to about 100 μg/patient.

A suitable dose for imaging medicine (using a paramagnetic imaging agent) will be in the range of from about 0.1 mg/patient to about 100 mg/patient, from about 0.5 to about 50 mg/patient or from 1 mg/patient to about 10 mg/patient.

For therapeutic applications, a suitable dose will be in the range of from about 0.05 picograms/kilogram to about 100 mg/kg, from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. The substance is conveniently administered in unit dosage form; for example, containing 5 to 1000 mg, conveniently 10 to 750 mg, most conveniently, 50 to 500 mg of active ingredient per unit dosage form.

Ideally, the substance should be administered to achieve peak plasma concentrations of from about 0.05 to about 100 μM, preferably, about 1 to 50 μM, most preferably, about 2 to about 30 μM. This may be achieved, for example, by the intravenous injection of a 0.005 to 10% solution of the substance, optionally in saline or orally administered as a bolus containing about 0.5-250 mg of the substance. Desirable blood levels may be maintained by continuous infusion to provide about 0.01-5.0 mg/kg/hr or by intermittent infusions containing about 0.4-15 mg/kg of the substance.

The substance may conveniently be presented in a single dose or as divided doses administered at appropriate intervals, for example, as two, three, four or more sub-doses per day.

The cobalamin conjugates may be administered orally in combination with a pharmaceutically acceptable vehicle such as an inert diluent or an edible carrier. They may be enclosed in hard or soft shell gelatin capsules, may be compressed into tablets or may be incorporated directly with the food of the patient's diet. For oral therapeutic administration, the substance may be combined with one or more excipients and used in the form of ingestible tablets, buccal tablets, troches, capsules, elixirs, suspensions, syrups, wafers and the like. Such compositions and preparations should contain at least 0.1% of the substance. The percentage of the compositions and preparations may, of course, be varied and may conveniently be between about 2 to about 60% of the weight of a given unit dosage form. The amount of substance in such therapeutically useful compositions is such that an effective dosage level will be obtained.

Tablets, troches, pills, capsules and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the substance may be incorporated into sustained-release preparations and devices.

Sublingual tablets are designed to dissolve very rapidly. Examples of such formulations include ergotamine tartrate, isosorbide dinitrate, isoproterenol HCl. The formulation of these tablets contain, in addition to the drug, a limited number of soluble excipients, usually lactose and powdered sucrose, but occasionally dextrose and mannitol. The process of making sublingual tablets involves moistening the blended powder components with an alcohol-water solvent system containing approximately 60% alcohol and 40% water.

In addition to the cobalamin conjugate, the prototype formulation for sublingual tablets may contain a binder such as povidone or HPMC, diluents such as lactose, mannitol, starch or cellulose, a disintegrant such as pregelatinized or modified starch, lubricants such as magnesium stearate, stearic acid or hydrogenated vegetable oil, a sweetener such as saccharin or sucrose and suitable flavoring and coloring agents.

XI. Controlled Release Formulations

The TC- or IF-binding agent and imaging agent is optionally administered in a controlled release formulation, which can be a degradable or nondegradable polymer, hydrogel or ganogel or other physical construct that modifies the bioabsorption, half life or biodegradation of the TC- or IF-binding agent/imaging agent. The controlled release formulation can be a material that is painted or otherwise applied onto the afflicted site, either internally or externally. In one embodiment, the invention provides a biodegradable bolus or implant that is inserted into the pocket created by surgical resection of a tumor or directly into the tumor itself. In another example, the controlled release formulation can be applied to a psoriatic lesion, eczema, atopic dermatitis, lichen planus, wart, pemphigus vulgaris, actinic keratosis, basal cell carcinoma or squamous cell carcinoma. The controlled release formulation can likewise be applied to a blood vessel to treat or prevent restenosis, retinopathies or atherosclerosis. The controlled release formulation with appropriated selected imaging agent can be used to coat a transplanted organ or tissue to prevent rejection. It can alternatively be implanted or otherwise applied near the site of rheumatoid arthritis.

The field of biodegradable polymers has developed rapidly since the synthesis and biodegradability of polylactic acid was first reported in 1966 by Kulkarni et al. “Polylactic acid for surgical implants,” Arch. Surg., 93, 839. Several other polymers are now known to biodegrade, such as polyanhydrides and polyorthoesters, which take advantage of labile backbone linkages (see: Domb et al. Macromolecules, 22, 3200, 1989; and Heller et al. Biodegradable Polymers as Drug Delivery Systems, Dekker, NY: 1990). Several polymers which degrade into naturally occurring materials have also been described, such as crosslinking gelatin, hyaluronic acid (della Valle et al. U.S. Pat. No. 4,987,744 and U.S. Pat. No. 4,957,744) and polyaminoacids (Miyake et al., 1974), which spurred the usage of polyesters by Holland et al. Controlled Release, 4, 155, 1986 and alph-hydroxy acids (i.e. lactic acid and glycolic acid), which remain the most widely used biodegradable materials for applications ranging from closure devices (sutures and staples) to drug delivery systems (Smith et al. U.S. Pat. No. 4,741,337; Spilizeqski et al. J. Control. Rel., 2, 197, 1985).

These polymers can be tailored to degrade at a desired rate and with a desired kinetics by selecting the appropriate monomers, method of preparation and molecular weight. Differences in crystallinity of the monomer can alter the polymeric degradation rate. Due to the relatively hydrophobic nature of most polymers, actual mass loss can begin with the oligomeric fragments that are small enough to be water soluble; hence, even the initial molecular weight can influence the degradation rate.

Hydrogels can be used in controlled release formulations. Such polymers are formed from macromers with a polymerizable, non-degradable, region that is separated by at least one degradable region. For example, the water soluble, non-degradable, region can form the central core of the macromer and have at least two degradable regions which are attached to the core, such that upon degradation, the non-degradable regions (in particular a polymerized gel) are separated. Specifically, as disclosed in U.S. Pat. No. 5,626,863 to Hubbell et al., the macromers are PEG-oligoglycolyl-acrylates, with the appropriate end caps to permit rapid polymerization and gelation. Acrylates can be polymerized readily by several initiating systems such as eosin dye, ultraviolet or visible light. The polyethyleneglycol (PEG) is highly hydrophilic and biocompatible. The oligoglycolic acid is a poly(a-hydroxy acid) which can be readily degraded by hydrolysis of the ester linkage into glycolic acid, a nontoxic metabolite. Other chain extensions include polylactic acid, polycaprolactone, polyorthoesters, polyanhydrides and polypeptides. This entire network can be gelled into a biodegradable network that can be used to entrap and homogeneously disperse water-soluble drugs for delivery at a controlled rate. Further, the gel can entrap particulate suspensions of water-insoluble drugs. (See also: U.S. Pat. No. 4,591,496 to Cohen et al. (Process for Making Systems for the Controlled Release of Macromolecules); U.S. Pat. No. 5,545,442 to Van Savage et al. (Method for Using a Radiation Cured Drug Release Controlling Membrane); U.S. Pat. No. 5,330,768 to Park et al. (Controlled Drug Delivery Using Polymer/Pluronic Blends); U.S. Pat. No. 5,122,367 to Ron et al. (Polyanhydride Bioerodible Controlled Release Inplants for Administration of Stabilized Growth Hormone); U.S. Pat. No. 5,545,409 to Laurencin et al. (Delivery System for Controlled Release of Bioactive Factors); U.S. Pat. No. 5,629,009 to Laurencin et al. (Delivery System for Controlled Release of Bioactive Factors).

Alternatively, delivery of biologically active substances, both in vitro and in vivo, via encapsulation has been well described in the prior art. U.S. Pat. No. 4,352,883 to Lim et al. entitled “Encapsulation of Biological Material” discloses the encapsulation of proteins within a membrane by suspending the protein in an aqueous medium containing a water-soluble gum that can be reversibly gelled to form the suspension into droplets. These droplets can be gelled further into discrete, shape-retaining, water insoluble temporary capsules with the aid of a solution of multivalent cations. The temporary capsules then can be further wrapped by an ionically cross-linking surface layer to form a semipermeable membrane around the capsules that is permeable to small molecules but impermeable to larger molecules. Microencapsulations of glycoproteins have also been well described. U.S. Pat. No. 4,324,683 to Lim et al. entitled “Encapsulation of Labile Biological Material” encapsulates a glycoprotein by a two-step interfacial polymerization process to form capsules with well-controlled porosity. The microcapsules serve to protect the active substances from attack by microorganisms and from any immunological response. U.S. Pat. No. 5,718,921 to Mathiowitz et al. (Microspheres Comprising Polymer and Drug Dispersed There Within) discloses a method to encapsulate relatively temperature-labile drugs into a microsphere.

Several methods have been developed to reversibly encapsulate biologically active substances, One that can be applied both to in vitro and in vivo studies has been described in U.S. Pat. No. 4,900,556 by Wheatley et al. entitled “System for Delayed and Pulsed Release of Biologically-Active Substances.” In this disclosed system, the biologically-active substance can be released either at a constant rate over a period of time or in discrete pulses. The biologically active materials are entrapped within liposomes encapsulated within semipermeable microcapsules or permeable polymeric matrix. Release of the desired materials is governed by the permeability of both the liposome and the surrounding matrix (the matrix integrity is directly proportional to the liposome integrity); the permeability of the liposome can be engineered by modifying the composition and the method for making the liposome to produce liposome that are sensitive to specific stimuli such as temperature, pH or light. For example, by including a phospholipase that degrades the liposome within some or all of the liposomes or the surrounding matrix, the liposome can be destabilized and broken down over a period of time. Other systems have been developed, e.g. U.S. Pat. No. 4,933,185 by Wheatley et al., which utilize a core made up of a polymer (such as an ionically cross-linked polysaccharide with calcium alginate or chitin) around which there is an ionically bound skin (such as a polycationic skin of poly-L-lysine) whose integrity is dependent on the core polymer. With an impermeable skin, when the core polymer can be degraded by enzymes (such as alginase from the bacteria, chitinase or hydrolase), there is a sudden release of biologically active substance from the core. Alternatively, the skin can be partially permeable for a gradual release of drug upon degradation of the core.

Nanoparticles are especially useful in the delivery of drugs parenterally or intravenously such that the delivery device is small with a long circulating half-life. A number of injectable drug delivery systems have been investigated, including microcapsules, microparticles, liposomes and emulsions. The major obstacle for these delivery systems is the rapid clearance of the materials from the blood stream by the macrophages of the reticuloendothelial system (RES). For example, polystyrene particles as small as sixty nanometers in diameter are cleared from the blood within two to three minutes. Liposomal drug delivery systems have also been extensively studied for this application because they were expected to freely circulate in the blood. Coating of the liposomes with poly(ethylene glycol) (PEG) increased the half-life of the carriers due to PEG's hydrophobic chains which reduced its protein absorption and thus its RES uptake. U.S. Pat. No. 5,543,158 to Gref et al. (Biodegradable Injectable Nanoparticles) describes a carrier system specifically targeted towards carriers suitable for intravenous delivery with a controlled release mechanism with modified polyglycols.

U.S. Pat. Nos. 5,626,862, 5,651,986 and 5,846,565 to Brem et al (Controlled Local Delivery of Chemotherapeutic Agents for Treating Solid Tumors) discloses the use of these carriers for the specific delivery of chemotherapeutic agents to increase bioavailability. Therefore, the devices act as reservoirs that release drugs over an extended period of time while at the same time preserves the bioactivity and bioavailability of the agent. U.S. Pat. No. 5,286,763 to Gerhard et al. (Bioerodible Polymers for Drug Delivery in Bone) further discloses that bioerodible polymers can be used to deliver chemotherapeutic agents directly into the bone. Cohen et al. U.S. Pat. No. 5,562,099 (Polymeric Microparticles Containing Agents for Inaging) discusses the usage of these carriers as contrast agents. The polymeric microparticle is filled with contrast agents for enhanced imaging.

Furthermore, U.S. Pat. No. 6,114,394 to Edwards, et al. (Polyamine Derivatives as Radioprotective Agents) discloses polyamine derivatives and the pharmaceutically acceptable addition salts thereof which are useful as radioprotective agents. The potential utility of these agents in protecting against exposure to environmental radiation, as well as in cancer radiation therapy, has long bee recognized. These agents, administered prior to or during exposure, would eliminate or reduce the severity of deleterious cellular effects caused by exposure to environmental ionizing radiation such as resulting from a nuclear explosion, a spill of radioactive material, close proximity to radioactive material and the like.

Books describing methods of controlled delivery that are appropriate for the delivery of the TC- or IF-binding agents/imaging agents of the present invention include: Robert S. Langer, Donald L. Wise, editors; Medical applications of controlled release (Volumes 1 and 2); Boca Raton, Fla.: CRC Press, 1984; and William J. M. Hrushesky, Robert Langer and Felix Theeuwes, editors; Temporal control of drug delivery (series); New York: N.Y. Academy of Sciences, 1991.

Nonlimiting examples of U.S. Patents that describe controlled release formulations are: U.S. Pat. No. 5,356,630 to Laurencin et al. (Delivery System for Controlled Release of Bioactive Factors); ; U.S. Pat. No. 5,797,898 to Santini, Jr. et al. (Microchip Drug Delivery Devices); U.S. Pat. No. 5,874,064 to Edwards et al. (Aerodynamically Light Particles for Pulmonary Drug Delivery); U.S. Pat. No. 5,548,035 to Kim et al. (Biodegradable Copolymer as Drug Delivery Matrix Comprising Polyethyleneoxide and Aliphatic Polyester Blocks); U.S. Pat. No. 5,532,287 to Savage et al. (Radiation Cured Drug Release Controlling Membrane); U.S. Pat. No. 5,284,831 to Kahl et al. (Drug Delivery Porphyrin Composition and Methods); U.S. Pat. No. 5,741,329 to Agrawal et al. (Methods of Controlling the pH in the Vicinity of Biodegradable Implants); U.S. Pat. No. 5,820,883 to Tice et al. (Methods for Delivering Bioactive Agents into and Through the Mucosally-Associated Lymphoid Tissues and Controlling Their Release); U.S. Pat. No. 5,955,068 to Gouin et al. (Biodegradable polyanhydrides Derived from Dimers of Bile Acids and Use Thereof as Controlled Drug Release Systems); U.S. Pat. No. 6,001,395 to Coombes et al. (Polymeric Lamellar Substrate Particles for Drug Delivery); U.S. Pat. No. 6,013,853 to Athanasiou et al. (Continuous Release Polymeric Implant Carriers); U.S. Pat. No. 6,060,582 to Hubbell et al. (Photopolymerizable Biodegradable Hydrogels as Tissue Contacting Materials and Controlled Release Carriers); U.S. Pat. No. 6,113,943 to Okada et al. (Sustained-Release Preparation Capable of Releasing a Physiologically Active Substance); and PCT Publication No. WO 99/59548 to Oh et al. (Controlled Drug Delivery System Using the Conjugation of Drug to Biodegradable Polyester); U.S. Pat. No. 6,123,861 (Fabrication of Microchip Drug Delivery Devices); U.S. Pat. No. 6,060,082 (Polymerized Liposomes Targeted to M cells and Useful for Oral or Mucosal Drug Delivery); U.S. Pat. No. 6,041,253 (Effect of Electric Field and Ultrasound for Transdermal Drug Delivery); U.S. Pat. No. 6,018,678 (Transdermal protein delivery or measurement using low-frequency sonophoresis); U.S. Pat. No. 6,007,845 Nanoparticles And Microparticles Of Non-Linear Hydrophilic-Hydrophobic Multiblock Copolymers; U.S. Pat. No. 6,004,534 Targeted Polymerized Liposomes For Inproved Drug Delivery; U.S. Pat. No. 6,002,961 Transdermal Protein Delivery Using Low-Frequency Sonophoresis; U.S. Pat. No. 5,985,309 Preparation Of Particles For Inhalation; U.S. Pat. No. 5,947,921 Chemical And Physical Enhancers And Ultrasound For Transdermal Drug Delivery; U.S. Pat. No. 5,912,017 Multiwall Polymeric Microspheres; U.S. Pat. No. 5,911,223 Introduction Of Modifying Agents Into Skin By Electroporation; U.S. Pat. No. 5,874,064 Aerodynamically Light Particles For Pulmonary Drug Delivery; U.S. Pat. No. 5,855,913 Particles Incorporating Surfactants For Pulmonary Drug Delivery; U.S. Pat. No. 5,846,565 Controlled Local Delivery Of Chemotherapeutic Agents For Treating Solid Tumors; U.S. Pat. No. 5,837,752 Semi-Interpenetrating Polymer Networks; U.S. Pat. No. 5,814,599 Transdermal Delivery Of Encapsulated Drugs; U.S. Pat. No. 5,804,178 Implantation Of Cell-Matrix Structure Adjacent Mesentery, Omentum Or Peritoneum Tissue; U.S. Pat. No. 5,797,898 Microchip Drug Delivery Devices; U.S. Pat. No. 5,770,417 Three-Dimensional Fibrous Scaffold Containing Attached Cells For Producing Vascularized Tissue In vivo; U.S. Pat. No. 5,770,193 Preparation Of Three-Dimensional Fibrous Scaffold For Attaching Cells To Produce Vascularized Tissue In vivo; U.S. Pat. No. 5,762,904 Oral Delivery Of Vaccines Using Polymerized Liposomes; U.S. Pat. No. 5,759,830 Three-Dimensional Fibrous Scaffold Containing Attached Cells For Producing Vascularized Tissue In vivo; U.S. Pat. No. 5,749,847 Delivery Of Nucleotides Into Organisms By Electroporation; U.S. Pat. No. 5,736,372 Biodegradable Synthetic Polymeric Fibrous Matrix Containing Chondrocyte For In vivo Production Of A Cartilaginous Structure; U.S. Pat. No. 5,718,921 Microspheres Comprising Polymer And Drug Dispersed There Within; U.S. Pat. No. 5,696,175 Preparation Of Bonded Fiber Structures For Cell Implantation; U.S. Pat. No. 5,667,491 Method For Rapid Temporal Control Of Molecular Transport Across Tissue; U.S. Pat. No. 5,654,381 Functionalized Polyester Graft Copolymers; U.S. Pat. No. 5,651,986 Controlled Local Delivery Of Chemotherapeutic Agents For Treating Solid Tumors; U.S. Pat. No. 5,629,009 Delivery System For Controlled Release Of Bioactive Factors; U.S. Pat. No. 5,626,862 Controlled Local Delivery Of Chemotherapeutic Agents For Treating Solid Tumors; U.S. Pat. No. 5,593,974 Localized Oligonucleotide Therapy; U.S. Pat. No. 5,578,325 Nanoparticles And Microparticles Of Non-Linear Hydrophilic-Hydrophobic Multiblock Copolymers; U.S. Pat. No. 5,562,099 Polymeric Microparticles Containing Agents For Imaging; U.S. Pat. No. 5,545,409 Delivery System For Controlled Release Of Bioactive Factors; U.S. Pat. No. 5,543,158 Biodegradable Injectable Nanoparticles; U.S. Pat. No. 5,514,378 Biocompatible Polymer Membranes And Methods Of Preparation Of Three Dimensional Membrane Structures; U.S. Pat. No. 5,512,600 Preparation Of Bonded Fiber Structures For Cell Implantation; U.S. Pat. No. 5,500,161 Method For Making Hydrophobic Polymeric Microparticles; U.S. Pat. No. 5,487,390 Gas-filled polymeric microbubbles for ultrasound imaging; U.S. Pat. No. 5,399,665 Biodegradable polymers for cell transplantation; U.S. Pat. No. 5,356,630 Delivery system for controlled release of bioactive factors; U.S. Pat. No. 5,330,768 Controlled drug delivery using polymer/pluronic blends; U.S. Pat. No. 5,286,763 Bioerodible polymers for drug delivery in bone; U.S. Pat. No. 5,149,543 Tonically cross-linked polymeric microcapsules; U.S. Pat. No. 5,128,420 Method of making hydroxamic acid polymers from primary amide polymers; U.S. Pat. No. 5,122,367 Polyanhydride bioerodible controlled release implants for administration of stabilized growth hormone; U.S. Pat. No. 5,100,668 Controlled release systems containing heparin and growth factors; U.S. Pat. No. 5,019,379 Unsaturated polyanhydrides; U.S. Pat. No. 5,010,167 Poly(amide-and imide-coanhydride) for biological application; U.S. Pat. No. 4,948,587 Ultrasound enhancement of transbuccal drug delivery; U.S. Pat. No. 4,946,929 Bioerodible articles useful as implants and prostheses having predictable degradation rates; U.S. Pat. No. 4,933,431 One step preparation of poly(amide-anhydride); U.S. Pat. No. 4,933,185 System for controlled release of biologically active compounds; U.S. Pat. No. 4,921,757 System for delayed and pulsed release of biologically active substances; U.S. Pat. No. 4,916,204 Pure polyanhydride from dicarboxylic acid and coupling agent; U.S. Pat. No. 4,906,474 Bioerodible polyanhydrides for controlled drug delivery; U.S. Pat. No. 4,900,556 System for delayed and pulsed release of biologically active substances; U.S. Pat. No. 4,898,734 Polymer composite for controlled release or membrane formation; U.S. Pat. No. 4,891,225 Bioerodible polyanhydrides for controlled drug delivery; U.S. Pat. No. 4,888,176 Controlled drug delivery high molecular weight polyanhydrides; U.S. Pat. No. 4,886,870 Bioerodible articles useful as implants and prostheses having predictable degradation rates; U.S. Pat. No. 4,863,735 Biodegradable polymeric drug delivery system with adjuvant activity; U.S. Pat. No. 4,863,611 Extracorporeal reactors containing immobilized species; U.S. Pat. No. 4,861,627 Preparation of multiwall polymeric microcapsules; U.S. Pat. No. 4,857,311 Polyanhydrides with improved hydrolytic degradation properties; U.S. Pat. No. 4,846,786 Bioreactor containing suspended, immobilized species; U.S. Pat. No. 4,806,621 Biocompatible, biocrodible, hydrophobic, implantable polyimino carbonate article; U.S. Pat. No. 4,789,724 Preparation of anhydride copolymers; U.S. Pat. No. 4,780,212 Ultrasound enhancement of membrane permeability; U.S. Pat. No. 4,779,806 Ultrasonically modulated polymeric devices for delivering compositions; U.S. Pat. No. 4,767,402 Ultrasound enhancement of transdermal drug delivery; U.S. Pat. No. 4,757,128 High molecular weight polyanhydride and preparation thereof; U.S. Pat. No. 4,657,543 Ultrasonically modulated polymeric devices for delivering compositions; U.S. Pat. No. 4,638,045 Non-peptide polyamino acid bioerodible polymers; U.S. Pat. No. 4,591,496 Process for making systems for the controlled release of macromolecules.

The invention may be further illustrated by the following examples.

### EXAMPLES

### Example 1

Preparation of Cyanocobalamin-b-(4-aminobutyl)amide

A mixture containing cyanocobalamin-b-carboxylic acid (1.0 g, 0.6 mmol), hydroxybenzotriazole (0.81 g, 6 mmol) and 1,4-diaminobutane dihydrochloride (4.8 g, 30 mmol) in 100 ml of water was adjusted to pH 7.8. 1- Ethyl-3-(3′-dimethylaminopropyl)carbodiimide (1.26 g, 6.6 mmol) was then added, the pH was adjusted to 6.4 and the reaction stirred at room temperature for 24 h. TLC on silica gel using n-butanol-acetic acid water (5:2:3) showed the reaction to be complete. Cyanocobalamin-b-(4-aminobutyl)amide was extracted into 92% aqueous phenol and the phenol layer was washed several times with equal volumes of water. To the phenol extract were added 3 volumes of diethylether and 1 volume of acetone. The desired cobalamin was removed from the organic phase by several extractions with water. The combined aqueous layers were extracted three times with diethylether to remove residual phenol, concentrated to approximately 200 ml in vacuo and crystallized from aqueous acetone. Yield 955 mg, 92%.

### Example 2

Proposed Preparation of Cyanocobalamin-b-(4-aminobutyl)amide-, Ciprofloxacin-, Levofloxacin-, Ofloxacin- and Sparfloxacin-Cobalamin Conjugates

A mixture containing cyanocobalamin-b-(4-aminobutyl)amide (0.6 mmol), hydroxybenzotriazole (6 mmol) and the antibiotic agent (e.g. Ciprofloxacin, Levofloxacin or Ofloxacin) (30 mmol) in 100 ml of water is adjusted to pH 7.8. 1-Ethyl-3-(3′-dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 3

Preparation of Methylcobalamin-b-(4-aminobutyl)amide

Methylcobalamin-b-carboxylic acid (1.0 g, 0.6 mmol) was reacted with diaminobutane dihydrochloride as described above for the cyano derivative. The cobalamin was purified by extraction through phenol (see above) and the resulting aqueous solution was concentrated in vacuo. This solution was chromatographed on AG1-X2 200-400 mesh in the acetate form (20.times.2.5 cm) and the pass through collected. The pass through was concentrated to approximately 20 ml and the desired cobalamin crystallized from aqueous acetone. Yield 920 mg, 88%. Unreacted methylcobalamin-b-carboxylic acid was eluted with 1M acetic acid, concentrated and crystallized from aqueous acetone. Yield 60 mg, 6%.

### Example 4

Proposed Preparation of Methylcobalamin-b-(4-aminobutyl)amide- Ciprofloxacin-, Levofloxacin-, Ofloxacin- and Sparfloxacin-Cobalamin Conjugates

A mixture containing methylcobalamin-b-(4-aminobutyl)amide (0.6 mmol), hydroxybenzotriazole (6 mmol) and the antibiotic agent (e.g. Ciprofloxacin, Levofloxacin or Ofloxacin) (30 mmol) in 100 ml of water is adjusted to pH 7.8. 1-Ethyl-3-(3′-dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 5

Preparation of Adenosylcobalamin-b-(4-aminobutyl)amide

Adenosylcobalamin-b-carboxylic acid (500 mg, 0.3 mmol) was reacted with diaminobutane dihydrochloride (2.4 mg, 15 mmol) as described above. The cobalamin was purified by extraction through phenol (see above). The resulting aqueous solution was concentrated in vacuo and applied to AG-50 X2, 200-400 mesh, in the hydrogen form (20.times.25 cm). The column was washed thoroughly with water to remove hydroxybenzotriazole and the desired cobalamin eluted with 1M ammonium hydroxide. After an additional extraction through phenol, adenosylcobalamin-b-(4-aminobutyl)amide was isolated as a glass. Yield 366 mg, 77%.

### Example 6

Proposed Preparation of Adenosylcobalamin-b-(4-aminobutyl)amide- Ciprofloxacin-, Levofloxacin-, Ofloxacin- and Sparfloxacin-Cobalamin Conjugates

A mixture containing adenosylcobalamin-b-(4-aminobutyl)amide (0.6 mmol), hydroxybenzotriazole (6 mmol) and the antibiotic agent (e.g. Ciprofloxacin, Levofloxacin or Ofloxacin) (30 mmol) in 100 ml of water is adjusted to pH 7.8. 1-Ethyl-3-(3′-dimethylaminopropyl)carbodiimide (6.6 mmol) is then added, the pH is adjusted to 6.4 and the reaction is stirred at room temperature for 24 h. TLC on silica gel using n-butanol-acetic acid water (5:2:3) shows the reaction to be complete. The product is extracted into 92% aqueous phenol and the phenol layer is washed several times with equal volumes of water. To the phenol extract is added 3 volumes of diethylether and 1 volume of acetone. The desired product is removed from the organic phase by several extractions with water. The combined aqueous layers are extracted three times with diethylether to remove residual phenol, concentrated to approximately 20 ml in vacuo and crystallized from aqueous acetone.

### Example 7

Preparation of Cyanocobalamin-b-(poly-L-lysine)amide-

Two preparations of -poly-L-lysine hydrobromide, one containing approximately 8 residues and a second one containing about 11 residues were separately reacted with cyanocobalamin-1-carboxylic acid. To each polymer (500 mg) dissolved in 20 mL of water was added 150 mg (0.1 mmol) of cyanocobalamin- 1-carboxylic acid, 338 mg (2.5 mmol) of hydroxybenzotriazole and 480 mg (2.5 mmol) of 1-ethyl-3(3-dimethyl-aminopropyl) carbodiimide. The pH was adjusted to 9 with IN NaOH and the reaction mixtures were stirred at room temperature for 2-3 h. They were purified on G-10 sephadex: the sizing columns (3×40 cm) were eluted with water and 1.5 mL fractions collected. The fractions showing the presence of the cobalamin (OD at 550 mm) and the presence of polylysine (ninhydrin positive) were pooled and freeze-dried.

### Example 8

Proposed Preparation of Cyanocobalamin-b-(polylysine)amide-, Ciprofloxacin-, Levofloxacin-, Ofloxacin- and Sparfloxacin-Conjugates

A mixture containing cyanocobalamin-b-(polylysine)amide (0.6 mmol), hydroxybenzotriazole (0.81 g, 6 mmol) and the antibiotic (e.g. Ciprofloxacin, Levofloxacin, Ofloxacin or Sparfloxacin) (30 mmol) in 100 ml of water is adjusted to pH 7.8. 1-Ethyl-3-(3′-dimethylaminopropyl)carbodiimide (1.26 g, 6.6 mmol) is then added, the pH is adjusted to 6.4 and the reaction is stirred at room temperature for 24 h. TLC on silica gel using n-butanol-acetic acid water (5:2:3) shows the reaction to be complete. The product is purified on G-10 sephadex; the sizing columns (3×40 cm) are eluted with water and 1-5 mL fractions are collected. The fractions showing the presence of cobalamin (OD at 550 mm) and the presence of polylysine (ninhydrin positive) are pooled and freeze-dried.

All publications, patents and patent documents are incorporated by reference herein, as though individually incorporated by reference. The invention has been described with reference to various specific and preferred embodiments and techniques. However, it should be understood that many variations and modifications may be made while remaining within the spirit and scope of the invention. In addition, some references were obtained on the World Wide Web (www). These references have been reproduced and are enclosed herein, as pages 69-86. These references are also incorporated by reference herein, as though individually incorporated by reference.

We claim:

1. A compound of the formula I: 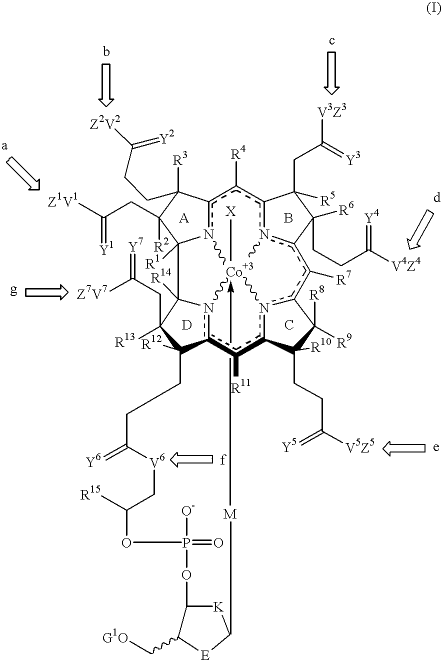
or its pharmaceutically acceptable salt, wherein:
a) the wavy line in the chemical structure indicates either a dative or covalent bond such that there are three dative Co—N bonds and one covalent Co—N bond, wherein, in the case of the dative bond, the valence of nitrogen is completed either with a double bond with an adjacent ring carbon or with a hydrogen;
b) the dotted line in the chemical structure indicates either a double or single bond such that the double bond does not over-extend the valence of the element (i.e. to give pentavalent carbons) and, in the case of a single bond, the valence is completed with hydrogen;
c) X is hydrogen, cyano, halogen (Cl, F, Br or I), haloalkyl, CF₃, CF₂CF₃, CH₂CF₃, CF₂Cl, NO, NO₂, NO₃, phosphonate, alkyl-P(O)₂OR¹⁵), PR¹⁵R¹⁶R¹⁷, NH₂, NR¹⁵R¹⁶, OH, OR¹⁵, SR¹⁵, SCN, N₃, OC(O)R¹⁵, C(O)₂R¹⁵, C(O)R¹⁵, OC(O)NR¹⁵R¹⁶, C(O)₂NR¹⁵R¹⁶, C(O)NR¹⁵R¹⁶, P(O)₂OR¹⁵, S(O)₂OR¹⁵, a purine or pyrimidine nucleoside or nucleoside analog, adenosyl, 5-FU, alkyl, alkenyl, alkynyl, aryl, aralkyl, alkaryl, amino acid, peptide, protein, carbohydrate, heteroalkyl, heterocycle, heteroaryl, alkylheteroaryl or L-T;
d) M is a monovalent heterocycle or heteroaromatic, which is capable of binding to the adjacent sugar ring;
e) K is O, S, NJ¹, C(OH)H, CR¹⁰⁰R¹⁰¹ or C(R¹⁰⁰)V⁸Z⁸;
f) E is O or S;
g) G¹ is hydrogen, alkyl, acyl, silyl, mono-, di- or tri-phosphate or L-T;
h) Y¹, Y², Y³, Y⁴, Y⁵, Y⁶ and Y⁷ independently are O, S or NJ²;
i) V¹, V², V³, V⁴, V⁵, V⁶, V⁷ and V⁸ independently are O, S, NJ³, CR¹⁰²R¹⁰³ or a direct bond;
j) Z¹, Z², Z³, Z⁴, Z⁵, Z⁷ and Z⁸ independently are R¹⁰⁴ or L-T;
k) each L is independently a direct bond or a linker to one or more T moieties and that does not significantly imnpair the ability of the TC- or IF-binding agent to bind to a transcobalamin receptor;
l) each T independently comprises an antibiotic agent, or a pharmaceutically acceptable residue thereof, optionally bound though a chelating moiety;
m) wherein at least one of Z¹, Z², Z³, Z⁴, Z⁵, Z⁷, Z⁸ and G¹ is independently L-T;
n) J¹, J² and J³ independently are hydrogen, alkyl, alkenyl, alkynyl, alkaryl, cycloalkyl, aryl, cycloaryl, heteroalkyl, heterocycle, heteroaryl, hydroxyl, alkoxy or amine;
o) R¹, R², R³, R⁴, R⁵, R⁶, R⁷, R⁸, R⁹, R¹⁰, R¹¹, R¹², R¹³ and R¹⁴ independently are hydrogen, lower alkyl, lower alkenyl, lower alkynyl, lower cycloalkyl, heteroalkyl, heterocyclic, lower alkoxy, azido, amino, lower alkylamino, halogen, thiol, SO₂, SO₃, carboxylic acid, C₁₋₆ carboxyl, hydroxyl, nitro, cyano, oxime or hydrazine;
p) R¹³ and R¹⁴ optionally can form a double bond;
q) R¹⁵, R¹⁶ and R¹⁷ are independently hydrogen, alkyl, alkenyl, alkynyl, aryl, alkaryl or aralkyl group, heteroalkyl, heterocycle or heteroaromatic; and
r) R¹⁰⁰, R¹⁰¹, R¹⁰², R¹⁰³ and R¹⁰⁴ are independently hydrogen, alkyl, alkenyl, alkynyl, aryl, acyl, heteroaromatic, heteroaryl, heteroalkyl, hydroxyl, alkoxy, cyano, azido, halogen, nitro, SO₂, SO₃, thioalkyl or amino.

2. The compound of claim 1 wherein
a) X is CN, OH, CH₃, adenosyl or L-T;
b) M is 5,6-dimethylbenzimidazole;
c) K is C(OH)H;
d) E is O;
e) G¹ is hydrogen, alkyl, acyl, silyl, mono-, di- or tri-phosphate or L-T
f) Y¹, Y², Y³, Y⁴, Y⁵, Y⁶ and Y⁷ are O;
g) V¹, V², V³, V⁴, V⁵, V⁶, V⁷ and V⁸ are independently NJ³;
h) R¹, R², R⁴, R⁵, R⁸, R⁹, R¹¹, R¹² and R¹⁵ are independently methyl;
i) R³, R⁶, R⁷, R¹⁰, R¹³ and R¹⁴ are independently hydrogen; and
j) Z¹, Z², Z³, Z⁴, Z⁵, Z⁷ and Z⁸ are independently hydrogen or L-T.

3. The compound of claim 1 wherein, M is a purine or pyrimidine.

4. The compound of claim 1 wherein M is 5,6-dimethylbenzimidazole.

5. The compound of claims 1 wherein, X is not L-T.

6. The compound of claim 1 wherein at least one of Z¹, Z², Z⁴ or Z⁵ is independently L-T.

7. The compound of claim 1 wherein at least two of Z¹, Z², Z⁴ or Z⁵ are independently L-T.

8. The compound of claim 6 or 7 wherein L is a bond.

9. The compound of claim 6 or 7 wherein L is not a bond.

10. The compound of claim 9 wherein at least one L is of the formula W—A—Q wherein A is (C₁-C₂₄)alkyl, (C₂-C₂₄)alkenyl, (C₂-C₂₄)alkynyl, (C₃-C₈)cycloalkyl, or (C₆-C₁₀)aryl, wherein W and Q are each independently —N(R)C(═O)—, —C(═O)N(R)—, —OC(═O)—, —C(═O)O—, —O—, —S—, —S(O)—, —S(O)₂—, —N(R)—, —C(═O)—, or a direct bond; wherein each R is independently H or (C₁-C₆)alkyl.

11. The compound of claim 10 wherein at least one of W and Q is independently —NR— or —COO—.

12. The compound of claim 10 wherein A is a (C₁-C₂₄)alkyl.

13. The compound of claim 9 wherein at least one L is about 5 angstroms to about 50 angstroms, in length, inclusive.

14. The compound of claim 9 wherein at least one L is a divalent radical formed from a peptide.

15. The compound of claim 9 wherein at least one L is a divalent radical formed from about 2-25 amino acids.

16. The compound of claim 14 or 15 wherein the divalent radical is a 1,ω-divalent radical.

17. The compound of claim 9 wherein at least one L is poly-L-glutamic acid, poly-L-aspartic acid, poly-L-histidine, poly-L-omithine, poly-L-serine, poly-L-threonine, poly-L-tyrosine, poly-L-leucine, poly-L-lysine-L-phenylalanine, poly-L-lysine or poly-L-lysine-L-tyrosine.

18. The compound of claim 17 wherein L is poly-L-lysine.

19. The compound of claim 18 wherein the poly-L-lysine contains about 8-11 residues.

20. The compound of claim 6 or 7 wherein Z¹ is L-T.

21. The compound of claim 6 or 7 wherein Z² is L-T.

22. The compound of claim 6 or 7 wherein Z⁴ is L-T.

23. The compound of claim 6 or 7 wherein Z⁵ is L-T.

24. The compound of claim 6 or 7 wherein Z² and Z⁴ are independently L-T.

25. The compound of claim 1 wherein at least one T is independently an aminoglycoside, β-lactam antibiotic, cephalosporin, macrolide, miscellaneous antibiotic, penicillin, tetracycline, antifungal, antimalarial agent, antituberculosis agent, antiviral, leprostatic, miscellaneous anti-infectives, quinolines, sulfonamide, urinary anti-infective, nasal antibiotic, opthalmic antibiotic, opthalmic antiviral, opthalmic quinoline, opthalmic sulfonamide, skin and mucous membrane antibiotic, skin and mucous membrane antifungal, skin and mucous membrane antiviral, skin and mucous membrane miscellaneous anti-infective, skin and mucous membrane scabicide or pedulicide, skin and mucous membrane anti-neoplast, nitrofiaran, or oxazolidinone, or a pharmaceutically acceptable residue thereof.

26. The compound of claim 1 wherein at least one T is independently Amikacin, Garamycin, Nebcin, Netromycin, Streptomycin Sulfate, TOBI, Azactam, Cefotan, Lorabid, Mefoxin, Merrem, Primaxin, Ancef, Ceclor, Cedax, Cefizox, Cefobid, Ceftin, Cefzil, Ceptaz, Claforan, Duricef, Fortaz, Keflex, Keftab, Kefurox, Kefzol, Mandol, Maxipime, Monocid, Omnicef, Rocephin, Suprax, Tazicef, Tazidime, Vantin, Zinacef, Cefpodoxime Proxetil, Cefprozil, Cephalexin Monohydrate, Biaxin, Dynabac, E.E.S. 200, E.E.S. 400, Ery-Ped 200 and EryPed 400, Ery-Tab, Erythrocin Stearate, Erythromycin, Ilosone, PCE Dispertab, Pedizole, Tao, Azithromycin, Clarithromycin, and Erythromycin, Cleocin HCl, Cleocin Phosphate, Coly-Mycin M, Seromycin, Vancocin HCl, Amoxil, Augmentin, Bicillin C-R 900/300, Bicillin C-R, Bicillin L-A, Geocillin, Mezlin, Omnipen, Pen-Vee K, Pfizerpen, Pipracil, Spectrobid, Ticar, Timentin, Unasyn, Zosyn, Amoxicillin/Clavulanic Acid, Amoxicillin Trihydrate, Ampicillin, Dicloxacillin Sodium, Penicillin V Potassium, Acbromycin, Declomycin, Dynacin, Minocin, Monodox, Terramycin, Vectrin, Vibramycin Calcium, Vibramycin Hyclate, Vibramycin Monohydrate, Vibra-Tabs, Demeclocycline HCL, Doxycycline, Minocyline HCL, Oxytetracycline HCL, Tetracycline, Lincomycins, Clindamycin, Abelcet, AmBisome, Amphotec, Ancobon, Diflucan, Fulvicin P/G, Fulvicin P/G 165 and 330, Grifulvin V, Gris-PEG, Lamisil, Nizoral, Amphotericin B, Clotrimazole, Dapsone, Fluconazole, Flucytosine, Miconazole, Nystatin, Sporanox, Aralen hydrochloride, Aralen phosphate, Daraprim, Lariam, Plaquenil, Capastat sulfate, Myambutol, Mycobutin, Nydrazid, Paser, Priftin, Pyrazinamide, Rifadin, Rifadin IV, Rifamate, Rifater, Seromycin, Tice BCG, Aminosalicylate Sodium, Cycloserine, Isoniazid, Rifampin, Methenamine, Trecator-SC, Alferon N, Crixivan, Cytovene, Cytovene-IV, Epivir, Famvir, Flumadine, Foscavir, Hivid, Intron A, Invirase, Norvir, Rebetron, Rescriptor, Retrovir, Retrovir IV, Symmetrel, Synagis, Valtrex, Videx, Viracept, Viramune, Virazole, Vistide, Zerit, Amantadine HCl, Lamiduvine, Zovirax, Dapsone, Daraprim, Flagyl 375, Flagyl, Furoxone, Mepron, Neutrexin, Cipro, Floxin, Levaquin, Mazaquin, Noroxin, Penetrex, Raxar, Trovan, Zagam, Bactrim, Bactrim DS, Pediazole, Septra, Septra DS, Co-Trimoxazole, Erythromycin/Sulfioxazole, Sulfadiazine, Sulfamethoxazole, Sulfapyridine, Sulfisoxazole, Furadantin, Macrobid, Macrodantin, Monurol, NegGram, Septra, Septra DS, Urised, Urobiotic-250, Uroqid, Vira-A, Chibroxin, Chibroxan, Blephamide, A/T/S, Benzamycin, Betadine, Cleocin T, Clindets, Emgel, Erycette, Klaron, Mycostatin, Theramycin Z, T-Stat, Terramycin, Exelderm, Fungizone, Lamisil, Loprox, Lotrimin, Lotrisone, Mentax, Monistat-Derm, Mycelex, Mycostatin, Naftin, Nizoral, Nystop, Oxistat, Selsun Rx, Spectazole, Denavir, Zovirax, Benzashave, Betadine, Betasept, Cetaphil, Clorpactin, Dapsone, Desquam-E, Desquam-X, Hibiclens, Hibistat, Impregon, MetroCream, MetroGel, Noritate, pHisoHex, Sulfacet-R, Sulfamylon, Triaz, Vanoxide-HC, Acticin, Elimite, Eurax, Lindane, Efudex, Fluoroplex, Zyvox, or Nitrofurantoin, or a pharmaceutically acceptable residue thereof.

27. The compound of claim 1 wherein at least one T is independently Ciprofloxacin, Levofloxacin, Ofloxacin or Sparfloxacin, or a pharmaceutically acceptable residue thereof.

28. The compound of claim 1 wherein the Z¹, Z², Z³, Z⁴, Z⁵, Z⁷, Z⁸ or G¹ moiety that is not L-T can further independently be L-T′ wherein T′ is an imaging agent, optionally bound through a chelating moiety.

29. The compound of claim 28 wherein the imaging agent is bound through a chelating moiety.

30. The compound of claim 29 wherein the chelating moiety is DPTA.

31. The compound of claim 29 wherein the imaging agent is a detectable radionuclide or a paramagnetic metal atom.

32. The compound of claim 31 wherein the detectable radionuclide or a paramagnetic metal atom is Technetium-99m, Indium-111 or Gadolinium-157.

33. The compound of claim 28, wherein the imaging agent is not bound through a chelating moiety.

34. The compound of claim 28 wherein the imaging agent is a non-metallic radionuclide.

35. The compound of claim 34 wherein the non-metallic radionuclide is carbon-11, fluorine-18, bromine-76, iodine-123 or iodine-124.

36. The compound of claim 28, wherein at least one of Z¹, Z², Z⁴ or Z⁵ is not L-T and is independently L-T′, wherein T′ is the imaging agent.

37. The compound of claim 37 wherein L is a bond.

38. The compound of claim 37 wherein L is not a bond.

39. A pharmaceutical composition for the treatment, prophylaxis or diagnosis of an infection in a host comprising the compound of any one of the preceding claims 1-38, or its pharmaceutically acceptable salt, together with a pharmaceutically acceptable carrier or diluent.

40. A pharmaceutical composition for the treatment, prophylaxis or diagnosis of an infection in a host comprising the compound of any one of the preceding claims 1-38, or its pharmaceutically acceptable salt, in combination with one or more antibiotic.

41. The composition of claim 40 wherein the antibiotic is an aminoglycoside, β-lactam antibiotic, cephalosporin, macrolide, miscellaneous antibiotic, penicillin, tetracycline, antifungal, antimalarial agent, antituberculosis agent, antiviral, leprostatic, miscellaneous anti-infectives, quinoline, sulfonamide, urinary anti-infective, nasal antibiotic, opthalmic antibiotic, opthalmic antiviral, opthalmic quinoline, opthaimic sulfonamide, skin and mucous membrane antibiotic, skin and mucous membrane antifungal, skin and mucous membrane antiviral, skin and mucous membrane miscellaneous anti-infective, skin and mucous membrane scabicide or pedulicide, skin and mucous membrane anti-neoplast, nitrofuran, or oxazolidinone, or a pharmaceutically acceptable residue thereof.

42. The composition of claim 40 wherein the antibiotic is Amikacin, Garamycin, Nebcin, Netromycin, Streptomycin Sulfate, TOBI, Azactam, Cefotan, Lorabid, Mefoxin, Merrem, Primaxin, Ancef, Ceclor, Cedax, Cefizox, Cefobid, Ceftin, Cefzil, Ceptaz, Claforan, Duricef, Fortaz, Keflex, Keftab, Kefurox, Kefzol, Mandol, Maxipime, Monocid, Onmicef, Rocephin, Suprax, Tazicef, Tazidime, Vantin, Zinacef, Cefpodoxime Proxetil, Cefprozil, Cephalexin Monohydrate, Biaxin, Dynabac, E.E.S. 200, E.E.S. 400, Ery-Ped 200 and EryPed 400, Ery-Tab, Erythrocin Stearate, Erythromycin, Ilosone, PCE Dispertab, Pedizole, Tao, Azithromycin, Clarithromycin, and Erythromycin, Cleocin HCl, Cleocin Phosphate, Coly-Mycin M, Seromycin, Vancocin HCl, Amoxil, Augmentin, Bicillin C-R 900/300, Bicillin C-R, Bicillin L-A, Geocillin, Mezlin, Omnipen, Pen-Vee K, Pfizerpen, Pipracil, Spectrobid, Ticar, Timentin, Unasyn, Zosyn, Amoxicillin/Clavulanic Acid, Amoxicillin Trihydrate, Ampicillin, Dicloxacillin Sodium, Penicillin V Potassium, Achromycin, Declomycin, Dynacin, Minocin, Monodox, Terramycin, Vectrin, Vibramycin Calcium, Vibramycin Hyclate, Vibramycin Monohydrate, Vibra-Tabs, Demeclocycline HCL, Doxycycline, Minocyline HCL, Oxytetracycline HCL, Tetracycline, Lincomycins, Clindamycin, Abelcet, AmBisome, Amphotec, Ancobon, Diflucan, Fulvicin P/G, Fulvicin P/G 165 and 330, Grifulvin V, Gris-PEG, Lamisil, Nizoral, Amphotericin B, Clotrimazole, Dapsone, Fluconazole, Flucytosine, Miconazole, Nystatin, Sporanox, Aralen hydrochloride, Aralen phosphate, Daraprim, Lariam, Plaquenil, Capastat sulfate, Myambutol, Mycobutin, Nydrazid, Paser, Priftin, Pyrazinamide, Rifadin, Rifadin IV, Rifamate, Rifater, Seromycin, Tice BCG, Aminosalicylate Sodium, Cycloserine, Isoniazid, Rifampin, Methenamine, Trecator-SC, Alferon N, Crixivan, Cytovene, Cytovene-IV, Epivir, Famvir, Flumadine, Foseavir, Hivid, Intron A, Invirase, Norvir, Rebetron, Rescriptor, Retrovir, Retrovir IV, Symmetrel, Synagis, Valtrex, Videx, Viracept, Viramune, Virazole, Vistide, Zerit, Amantadine HCl, Lamiduvine, Zovirax, Dapsone, Daraprim, Flagyl 375, Flagyl, Furoxone, Mepron, Neutrexin, Cipro, Floxin, Levaquin, Mazaquin, Noroxin, Penetrex, Raxar, Trovan, Zagam, Bactrim, Bactrim DS, Pediazole, Septra, Septra DS, Co-Trimoxazole, Erythromycin/Sulfioxazole, Sulfadiazine, Sulfamethoxazole, Sulfapyridine, Sulfisoxazole, Furadantin, Macrobid, Macrodantin, Monurol, NegGram, Septra, Septra DS, Urised, Urobiotic-250, Uroqid, Vira-A, Chibroxin, Chibroxan, Blephamide, A/T/S, Benzamycin, Betadine, Cleocin T, Clindets, Emgel, Erycette, Klaron, Mycostatin, Theramycin Z, T-Stat, Terramycin, Exelderm, Fungizone, Lamisil, Loprox, Lotrimin, Lotrisone, Mentax, Monistat-Derm, Mycelex, Mycostatin, Naftin, Nizoral, Nystop, Oxistat, Selsun Rx, Spectazole, Denavir, Zovirax, Benzashave, Betadine, Betasept, Cetaphil, Clorpactin, Dapsone, Desquam-E, Desquam-X, Hibiclens, Hibistat, Impregon, MetroCream, MetroGel, Noritate, pHisoHex, Sulfacet-R, Sulfamylon, Triaz, Vanoxide-HC, Acticin, Elimite, Eurax, Lindane, Efudex, Fluoroplex, Zyvox, or Nitrofurantoin, or a pharmaceutically acceptable residue thereof.

43. The composition of claim 40 wherein the antibiotic is Ciprofloxacin, Levofloxacin, Ofloxacin or Sparfloxacin, or a pharmaceutically acceptable residue thereof.

44. A method for the treatment or prophylaxis of an infection in a host, comprising administering a therapeutic amount of the compound of any one of the preceding claims 1-38, or its pharmaceutically acceptable salt therein, which comprises an antibiotic.

45. A method for the treatment, prophylaxis and/or diagnosis of an infection in a host, comprising administering an effective amount of the compound of any one of the preceding claims 1-38, or its pharmaceutically acceptable salt therein, which comprises an antibiotic and/or an imaging agent, and optionally detecting the presence of the compound.

46. A method for the diagnosis of an infection in a host, comprising administering to the animal a detectable amount of the compound of any one of the preceding claims 28-38, or its pharmaceutically acceptable salt therein, which comprises an imaging agent and detecting the presence of the compound.

47. A method for the treatment or prophylaxis of an infection in a host, comprising administering a therapeutic amount of a pharmaceutical composition comprising the compound of any one of the preceding claims 1-38, which is contains at least one antibiotic, or its pharmaceutically acceptable salt therein, and a pharmaceutically acceptable carrier.

48. A method for the treatment, prophylaxis and/or diagnosis of an infection in a host, comprising administering an effective amount of a pharmaceutical composition comprising the compound of any one of the preceding claims 1-38, linked to at least one antibiotic and/or imaging agent, or its pharmaceutically acceptable salt therein, and a pharmaceutically acceptable carrier, and optionally detecting the presence of the compound.

49. A method for the diagnosis of an infection in a host, comprising administering a detectable amount of a pharmaceutical composition comprising the compound of any one of the preceding claims 28-38, linked to at least one imaging agent, or its pharmaceutically acceptable salt therein, and a pharmaceutically acceptable carrier, and detecting the presence of the compound.

50. The method of any one of claims 44-49 wherein the infection is an acute lower respiratory infection, lower urinary tract infection, tuberculosis, Lyme's disease, malaria, meningitis, meningitis caused by Neisseria meningitis, hepatitis, measles, neonatal tetanus, diarrheal disease, whooping cough, intestinal worm disease, sexually transmitted diseases, or any combination thereof.
